(19) United States
(12) Patent Application Publication
(10) Pub. No.: US 2003/0191121 A1
(43) Pub. Date: Oct. 9, 2003

(54) Piperazine carboxamide intermediates of HIV protease inhibitors and processes for their preparation

(76) Inventors: Ross A. Miller, Fanwood, NJ (US); Sandor Karady, Mountainside, NJ (US); Paul J. Reider, Westfield, NJ (US)

Correspondence Address: MERCK AND CO INC, P O BOX 2000, RAHWAY, NJ, 070650907

(21) Appl. No.: 10/212,978

(22) Filed: Aug. 6, 2002

Related U.S. Application Data
(60) Provisional application No. 60/311,016, filed on Aug. 9, 2001.
(60) Provisional application No. 60/327,349, filed on Oct. 5, 2001.

(51) Int. Cl. A61K 31/541; A61K 31/5377; A61K 31/498; C07D 487/02

(52) U.S. Cl. 514/228.5; 514/234.2; 514/249; 544/60; 544/117; 544/350

(57) ABSTRACT

Piperazine carboxamide intermediates of HIV protease inhibitors and a process for their preparation are disclosed. The piperazine carboxamide compounds are of Formula (III):

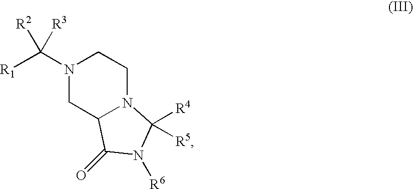

wherein R¹, R², R³, R⁴, R⁵, and R⁶ are defined herein. The process for preparing the intermediates comprises coupling an iminium salt of Formula I:

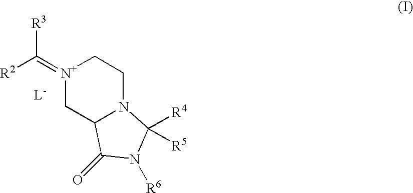

with a metallated derivative of a compound of Formula (II):

R¹—H (II),

wherein L⁻ is a counterion. A process for preparing the iminium salt of Formula (I) is also disclosed, as is a process for preparing HIV protease inhibitors from Compound III.

### FIELD OF THE INVENTION

The present invention is directed to piperazine carboxamide compounds and processes for making the compounds. The piperazine carboxamides are useful as intermediates for making HIV protease inhibitors, including protease inhibitors which are very potent against HIV viral mutants.

### BACKGROUND OF THE INVENTION

The HIV retrovirus is the causative agent for AIDS. The HIV-1 retrovirus primarily uses the CD4 receptor (a 58 kDa transmembrane protein) to gain entry into cells, through high-affinity interactions between the viral envelope glycoprotein (gp120) and a specific region of the CD4 molecule found in T-lymphocytes and CD4 (+) T-helper cells (Lasky L. A. et al., Cell 1987, 50: 975-985). HIV infection is characterized by an asymptomatic period immediately following infection that is devoid of clinical manifestations in the patient. Progressive HIV-induced destruction of the immune system then leads to increased susceptibility to opportunistic infections, which eventually produces a syndrome called ARC (AIDS-related complex) characterized by symptoms such as persistent generalized lymphadenopathy, fever, and weight loss, followed itself by full blown AIDS.

After entry of the retrovirus into a cell, viral RNA is converted into DNA, which is then integrated into the host cell DNA. The reverse transcriptase encoded by the virus genome catalyzes the first of these reactions (Haseltine W. A. FASEB J. 1991, 5: 2349-2360). At least three functions have been attributed to the reverse transcriptase: RNA-dependent DNA polymerase activity which catalyzes the synthesis of the minus strand DNA from viral RNA, ribonuclease H(RNase H) activity which cleaves the RNA template from RNA-DNA hybrids and DNA-dependent DNA polymerase activity which catalyzes the synthesis of a second DNA strand from the minus strand DNA template (Goff S. P., J. Acq. Imm. Defic. Syndr. 1990, 3: 817-83 1). The double stranded DNA produced by reverse transcriptase, now called provirus, is then able to be inserted into host genomic DNA.

At the end of reverse transcription, the viral genome in the form of DNA is integrated into host genomic DNA and serves as a template for viral gene expression by the host transcription system, which leads eventually to virus replication (Sakai, H al., J. Virol. 1993, 67: 1169-1174). The preintegration complex consists of integrase, reverse transcriptase, p 17 and proviral DNA (Bukrinsky et al., Proc. Nat. Acad. Sci. USA 1992, 89: 6580-6584). The phosphorylated p17 protein plays a key role in targeting the preintegration complex into the nucleus of host cell (Gallay et al., Cell 1995, 80:, 379-388).

As in the case of several other retroviruses, HIV encodes the production of a protease which carries out post-translational cleavage of precursor polypeptides in a process necessary for the formation of infectious virions (S. Crawford et al., J. Virol. 1985, 53: 899). These gene products include pol—which encodes the virion RNA-dependent DNA polymerase (reverse transcriptase), an endonuclease, and HIV protease—and gag—which encodes the core-proteins of the virion. (H. Toh et al., EMBO J. 1985, 4: 1267; L. H. Pearl et al., Nature 1987, 329-351; M. D. Power et al., Science 1986, 231: 1567).

A number of synthetic anti-viral agents targeted to various stages in the replication cycle of HIV have been disclosed. These agents include inhibitors of HIV cellular fusion (Turpin et al., Expert Opinion on Therapeutic Patents 2000, 10: 1899-1909), reverse transcriptase inhibitors (e.g., didanosine, zidovudine (AZT), and efavirenz), integrase inhibitors (Neamati, Expert Opinion on Investigational Drugs 2000, 10: 281-296), and protease inhibitors. Protease inhibitors inhibit the formation of infectious virions by interfering with the processing of viral polyprotein precursors. Processing of these precursor proteins requires the action of virus-encoded proteases which are essential for replication (Kohl, N. E. et al., Proc. Natl. Acad. Sci. USA 1988, 85: 4686).

Several HIV protease inhibitors are presently in clinical use for the treatment of AIDS, ARC and HIV infection, including indinavir (see U.S. Pat. No. 5,413,999), nelfinavir (U.S. Pat. No. 5,484,926), saquinavir (U.S. Pat. No. 5,196,438), and ritonavir (U.S. Pat. No. 5,484,801). Each of these protease inhibitors is a peptidomimetic, competitive inhibitor of the viral protease which prevents cleavage of the HIV gag-pol polyprotein precursor. Indinavir, for example, has been found to be highly effective in reducing HIV viral loads and increasing CD4 cell counts in HIV-infected patients, when used in combination with nucleoside reverse transcriptase inhibitors. See, for example, Hammer et al., New England J. Med. 1997, 337: 725-733 and Gulick et al., New England J. Med. 1997, 337: 734-739.

A substantial and persistent problem in the treatment of AIDS has been the ability of the HIV virus to develop resistance to the therapeutic agents employed to treat the disease. Resistance to HIV-1 protease inhibitors has been associated with 25 or more amino acid substitutions in both the protease and the cleavage sites. Many of these viral variants are resistant to all of the HIV protease inhibitors currently in clinical use. See Condra et al., Drug Resistance Updates 1998, 1: 1-7; Condra et al., Nature 1995, 374: 569-571; Condra et al., J. Virol. 1996, 70: 8270-8276; Patrick et al., Antiviral Ther. 1996, Suppl. 1: 17-18; and Tisdale et al., Antimicrob. Agents Chemother. 1995, 39: 1704-1710.

WO 01/38332 describes a class of γ-hydroxy-2-(fluoroalkylaminocarbonyl)-1-piperazinepentanamide compounds which are HV protease inhibitors that are much more potent against mV viral mutants than protease inhibitors presently in clinical use. The synthesis of many of these protease inhibitors as described in WO 01/38332 is a complicated, multi-step process having a relatively low overall yield. The synthesis of the compounds of this class of protease inhibitors containing oxazolylalkyl substituents is representative of the preparative chemistry disclosed in WO '332 and is shown in Scheme A:

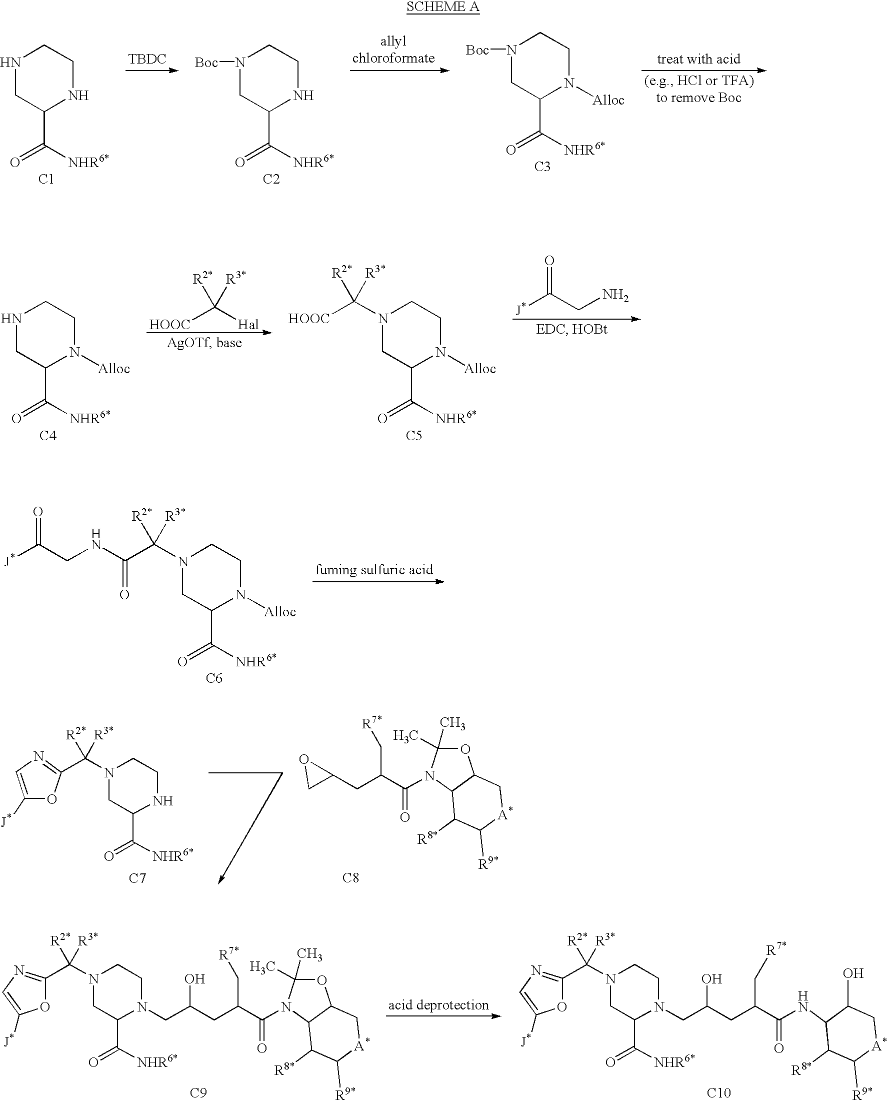

R²*, R³*=H or alkyl; or

R²* and R³* together with the carbon to which they are attached form cycloalkyl;

R⁶*=monofluoroalkyl or polyfluoroalkyl;

R⁷*=alkyl, cycloalkyl, aryl or heteroaryl, wherein aryl is optionally substituted with one or more of halogen, OH, alkyl, alkenyl, alkynyl, fluoroalkyl, —O-alkyl, or heteroaryl; and heteroaryl is optionally substituted with one or more of halogen, OH, alkyl, alkenyl, alkynyl, fluoroalkyl, —O-alkyl, or aryl;

R⁸*, R⁹*=H, OH, alkyl, fluoroalkyl, or —O-alkyl; or

R⁸* and R⁹* together with the carbons to which they are attached form a fused benzene ring;

A* absent, CH₂, or O;

J*=aryl or heteroaryl, wherein aryl is optionally substituted with one or more of halogen, hydroxy, cyano, alkyl, fluoroalkyl, —O-alkyl, —O-fluoroalkyl, S-alkyl, amino, or heteroaryl; and heteroaryl is optionally substituted with one or more of halogen, hydroxy, cyano, alkyl, fluoroalkyl, —O-alkyl, —O-fluoroalkyl, S-alkyl, amino, aryl, or heteroaryl.

There is a need for alternative methods of preparing these protease inhibitors that require fewer steps and provide higher overall yields.

### SUMMARY OF THE INVENTION

The present invention is directed to piperazine carboxamide intermediates of HIV protease inhibitors and processes for their preparation. More specifically, the present invention includes a compound of Formula (III):

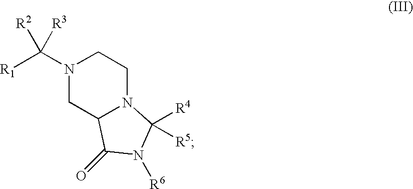

wherein

R¹ is C₁-C₆ alkyl, C₂-C₆ alkenyl, C₂-C₆ alkynyl, C₃-C₈ cycloalkyl, aryl, or heteroaryl; wherein the alkyl, alkenyl, alkynyl, cycloalkyl, aryl or heteroaryl is optionally substituted with one or more substituents each of which is independently:

(1) cyano,

(2) C₁-C₆ alkyl,

(3) C₂-C₆ alkenyl,

(4) C₂-C₆ alkynyl,

(5) —O—C₁-C₆ alkyl,

(6) —S—C₁-C₆ alkyl,

(7) —N(R^{a})(SO₂R^{b}),

(8) —NR^{c}R^{d},

(9) —C(═O)—NR^{c}R^{d};

(10) phenyl,

(11) phenyl substituted with one or more substituents each of which is independently halogen, cyano, C₁-C₆ alkyl, C₂-C₆ alkenyl, C₂-C₆ alkynyl, —O—C₁-C₆ alkyl, or S—C₁-C₆ alkyl,

(12) heterocycle, or

(13) heterocycle substituted with one or more substituents each of which is independently cyano, C₁-C₆ alkyl, C₂-C₆ alkenyl, C₂-C₆ alkynyl, —O—C₁-C₆ alkyl, S—C₁-C₆ alkyl, NR^{c}R^{d}, or a 5- or 6-membered heteroaromatic ring consisting of from 1 to 3 heteroatoms selected from N, O and S and a balance of carbon atoms;

R² and R³ are each independently hydrogen, C₁-C₆ alkyl, or aryl, wherein the alkyl group is optionally substituted with one or more substituents each of which is independently halogen, —O—C₁-C₆ alkyl, or —O—C₁-C₆ haloalkyl; and wherein the aryl group is optionally substituted with one or more substituents each of which is independently halogen, —C₁-C₆ alkyl, —C₁-C₆ haloalkyl, —O—C₁-C₆ alkyl, or —O—C₁-C₆ haloalkyl; or

R² and R³ together with the carbon to which they are attached form C₃-C₈ cycloalkyl which is optionally substituted with one or more substituents each of which is independently halogen, —C₁-C₆ alkyl, —C₁-C₆ haloalkyl, —O—C₁-C₆ alkyl, —O—C₁-C₆ haloalkyl, or —C₁-C₆ alkyl-OR^{e};

R⁴ and R⁵ are each independently

(1) —H,

(2) halogen,

(3) —C₁-C₆ alkyl which is optionally substituted with one or more substituents each of which is independently:

(a) halogen,

(b) —O—C₁-C₆ alkyl,

(c) —O—C₁-C₆ haloalkyl,

(d) —C₁-C₆ alkyl-OR^{e}, or

(e) —N(Re)₂,

(4) aryl which is optionally substituted with one or more substituents each of which is independently:

(a) halogen,

(b) —O—C₁-C₆ alkyl,

(c) —O—C₁-C₆ haloalkyl,

(d) —C₁-C₆ alkyl-OR^{e}, or

(e) —N(Re)₂,

(5) heteroaryl which is optionally substituted with one or more substituents each of which is independently:

(a) halogen,

(b) —O—C₁-C₆ alkyl,

(c) —O—C₁-C₆ haloalkyl,

(d) —C₁-C₆ alkyl-OR^{e}, or

(e) —N(Re)₂,

or R⁴ and R⁵ together with the carbon atom to which they are attached form:

(i) C₃-C₈ cycloalkyl which is optionally substituted with one or more substituents each of which is independently halogen, —C₁-C₆ alkyl, —C₁-C₆ haloalkyl, —O—C₁-C₆ alkyl, —O—C₁-C₆ haloalkyl, or —C₁-C₆ alkyl-OR^{e}, or

(ii) a group of formula:

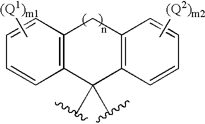

wherein each Q¹ and Q² is independently halogen, —C₁-C₆ alkyl, —C₁-C₆ haloalkyl, —O—C₁-C₆ alkyl, or —O—C₁-C₆ haloalkyl;

m1 and m2 are each independently integers equal to zero, 1, 2, 3 or 4; and

n is an integer equal to zero, 1 or 2;

R⁶ is —H or C₁-C₆ alkyl optionally substituted with one or more substituents each of which is independently

(1) halogen,

(2) —O—C₁-C₆ alkyl,

(3) —O—C₁-C₆ haloalkyl,

(4) —C₁-C₆ alkyl-OR^{e},

(5) —N(R^{e})₂,

(6) —CO₂R^{e},

(7) —N(R^{e})(SO₂R^{e}),

(8) —C(═O)R^{e}, or

(9) —C(═O)—N(R^{e})₂;

each R^{a} is independently —H or —C₁-C₄ alkyl;

each R^{b} is independently —H or —C₁-C₄ alkyl;

R^{c} and R^{d} are each independently —H or —C₁-C₄ alkyl; or alternatively R^{c} and R^{d} together with the nitrogen to which they are attached form C₃-C₆ azacycloalkyl; and

each R^{e} is independently a —C₁-C₄ alkyl;

or a salt thereof.

The present invention also includes a process for preparing a compound of Formula (III), which comprises coupling an iminium salt of Formula I:

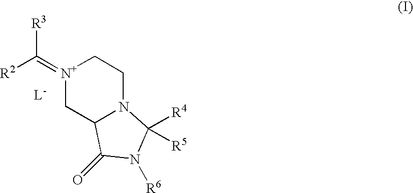

with a metallated derivative of a compound of Formula (H):

R¹—H (II),

in solvent to obtain Compound III;

wherein L⁻ is a counterion; and R¹, R², R³, R⁴, R⁵, and R⁶ are each as defined above.

The present invention also includes the iminium salt of Formula (I) and a process for its preparation. The present invention further includes processes for preparing HIV protease inhibitors which incorporate steps involving Compound III and optionally Compound I, which processes require significantly fewer steps and are characterized by substantially higher overall yields than earlier processes such as that shown in Scheme A above. An important feature of intermediates I and III is the presence of the

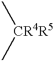

protecting group on the proximal nitrogen of the piperazine carboxamide, leaving the distal nitrogen available for coupling. This selective protective group can be introduced in a single step using an inexpensive, readily available reagent such as acetone, and later removed by simple acidification, as described below. By contrast, previous preparative methods (e.g., Scheme A) involved the introduction of two different protective groups in two separate steps, which were followed later by their removal in two separate deprotection steps.

Other embodiments, aspects and features of the present invention are either further described in or will be apparent from the ensuing description, examples and appended claims.

### DETAILED DESCRIPTION OF THE INVENTION

The present invention includes the nitrogen-protected piperazine carboxamide compound of Formula III or a salt thereof, as set forth above in the Summary of the Invention. Suitable salts of Compound III include include the conventional salts formed from inorganic or organic acids. In an aspect of the invention, the salts are non-toxic salts.

One embodiment of the present invention is a compound of Formula III, wherein R¹ is C₁-C₆ alkyl, C₃-C₈ cycloalkyl, aryl, or heteroaryl, wherein heteroaryl is (i) a 5- or 6-membered aromatic ring consisting of from 1 to 3 heteroatoms selected from N, S, and O and a balance of carbon atoms or (ii) an 8- to 10-membered bicyclic ring system consisting of from 1 to 3 heteroatoms selected from N, S, and O and a balance of carbon atoms, wherein at least one of the rings in the bicyclic system is an aromatic ring; and wherein the alkyl, cycloalkyl, aryl or heteroaryl is optionally substituted with one or more substituents each of which is independently:

(1) cyano,

(2) C₁-C₆ alkyl,

(3) C₂-C₆ alkenyl,

(4) C₂-C₆ alkynyl,

(5) —O—C₁-C₆ alkyl,

(6) S—C₁-C₆ alkyl,

(7) —N(R^{a})(SO₂R^{b}),

(8) —NR^{c}R^{d},

(9) —C(═O)—NR^{c}R^{d};

(10) phenyl,

(11) phenyl substituted with one or more substituents each of which is independently halogen, cyano, C₁-C₆ alkyl, C₂-C₆ alkenyl, C₂-C₆ alkynyl, —O—C₁-C₆ alkyl, or S—C₁-C₆ alkyl,

(12) heterocycle, or

(13) heterocycle substituted with one or more substituents each of which is independently cyano, C₁-C₆ alkyl, C₂-C₆ alkenyl, C₂-C₆ alkynyl, —O—C₁-C₆ alkyl, S—C₁-C₆ alkyl, NR^{c}R^{d}, or a 5- or 6-membered heteroaromatic ring consisting of from 1 to 3 heteroatoms selected from N, O and S and a balance of carbon atoms;

and all other variables are as originally defined;

or a salt thereof.

Another embodiment of the present invention is a compound of Formula III, wherein R¹ is C₁-C₆ alkyl, C₁-C₆ cycloalkyl, phenyl, or heteroaryl; wherein heteroaryl is pyridyl, methylenedioxyphenyl, furanyl, benzofuranyl, benzothiofuranyl, benzoxazolyl, benzothiazolyl, azabenzothiazolyl, azabenzoxazolyl, azabenzofuranyl, azabenzothiofuranyl, oxazolyl, thiazolyl, isoxazolyl, isothiazolyl, imidazolyl, triazolyl, thiadiazolyl, oxadiazolyl, indazolyl, pyrrolyl, pyrazolyl, thiophenyl, or thienothiophenyl; and wherein the alkyl, cycloalkyl, aryl or heteroaryl is optionally substituted with one or more substituents each of which is independently:

(1) cyano,

(2) C₁-C₄ alkyl,

(3) —O—C₁-C₄ alkyl,

(4) S—C₁-C₄ alkyl,

(5) NR^{c}R^{d},

(6) phenyl,

(7) phenyl substituted with one or more substituents each of which is independently halogen, cyano, C₁-C₄ alkyl, —O—C₁-C₄ alkyl, or S—C₁-C₄ alkyl,

(8) heterocycle which is a 5- or 6-membered unsaturated monocyclic ring consisting of from 1 to 3 heteroatoms selected from N, O and S and a balance of carbon atoms, or

(9) heterocycle which is a 5- or 6-membered unsaturated monocyclic ring as defined in (12) substituted with one or more substituents each of which is independently cyano, C₁-C₄ alkyl, —O—C₁-C₄ alkyl, S—C₁-C₄ alkyl, NR^{c}R^{d} thiazolyl, oxazolyl, imidazolyl, pyrazolyl, triazolyl, pyrrolyl, furanyl, thienyl, isoxazolyl, and isothiazolyl;

and all other variables are as originally defined;

or a salt thereof.

Still another embodiment of the present invention is a compound of Formula III, wherein R¹ is

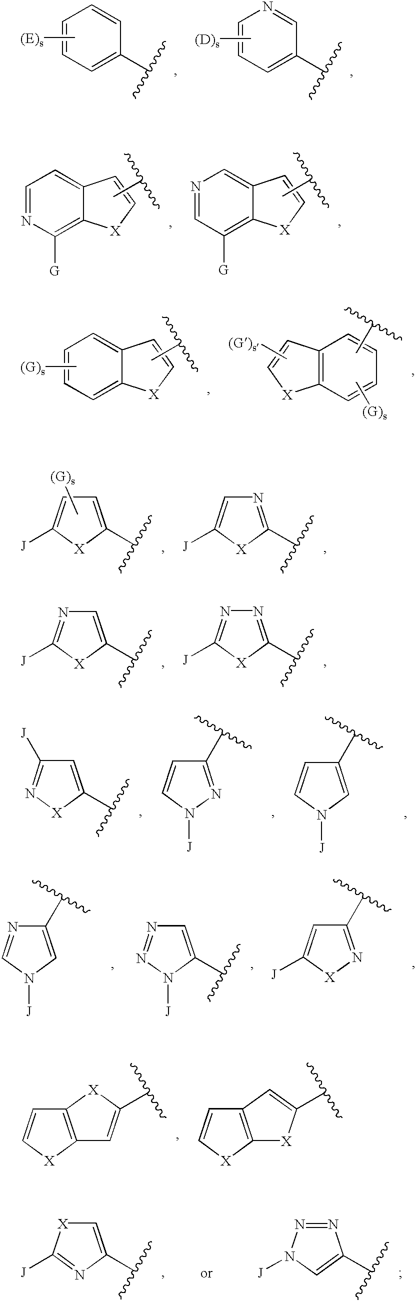

each D is independently hydrogen, cyano, NR^{c}R^{d}, C₁-C₄ alkyl, —O—C₁-C₄ alkyl, S—C₁-C₄ alkyl, phenyl, substituted phenyl, heterocycle, or substituted heterocycle; wherein substituted phenyl is phenyl with one or more subsituents independently selected from halogen, hydroxy, C₁-C₄ alkyl, and —O—C₁-C₄ alkyl; and wherein substituted heterocycle is heterocycle with one or more substituents independently selected from C₁-C₄ alkyl, —O—C₁-C₄ alkyl, and S—C₁-C₄ alkyl;

each E is independently hydrogen, cyano, C₁-C₄ alkyl, —O—C₁-C₄ alkyl, heterocycle, or substituted heterocycle;

G and G′ are each independently selected from hydrogen, halogen, cyano, hydroxy, C₁-C₄ alkyl, C₁-C₄ fluoroalkyl, and —O—C₁-C₄ alkyl;

J is

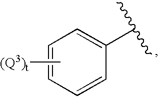

heterocycle, or substituted heterocycle;

each Q³ is independently hydrogen, halogen, cyano, C₁-C₄ alkyl, or —O—C₁-C₄ alkyl;

X is O or S;

heterocycle in each of D, E and J is independently

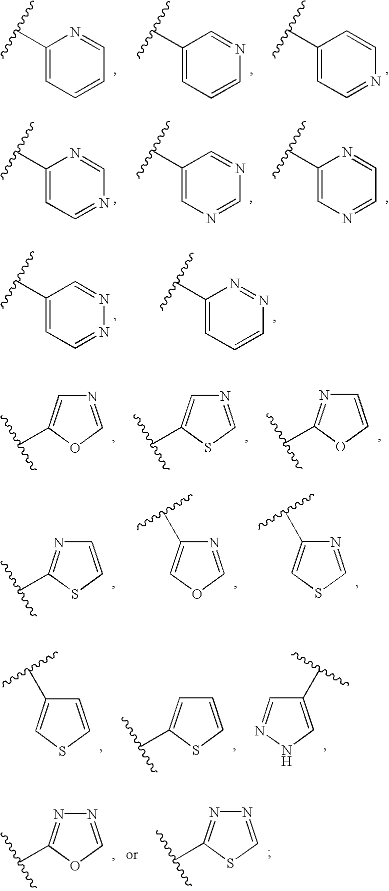

substituted heterocycle in each of E and J is independently heterocycle as defined above with one or more substituents independently selected from cyano, C₁-C₄ alkyl, —O—C₁-C₄ alkyl, S—C₁-C₄ alkyl, NR^{c}R^{d}, thiazolyl, oxazolyl, imidazolyl, pyrazolyl, triazolyl, pyrrolyl, isoxazolyl, and isothiazolyl;

s, s′, and t are each independently integers from 0 to 2;

and all other variables are as originally defined;

or a salt thereof.

In an aspect of the preceding embodiment, R¹ is

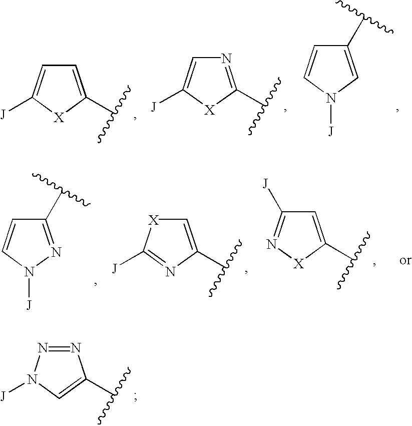

wherein J is

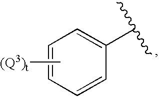

heterocycle, or substituted heterocycle;

each Q³ is independently hydrogen, halogen, cyano, C₁-C₄ alkyl, or —O—C₁-C₄ alkyl;

heterocycle is

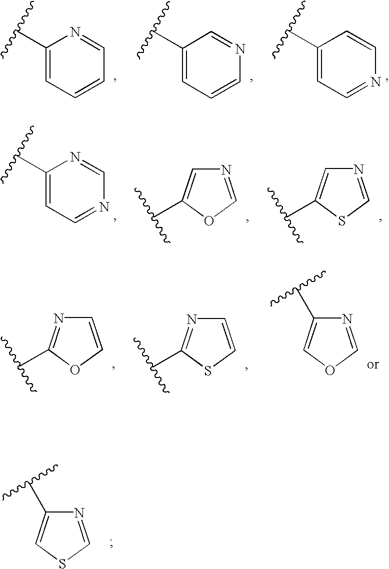

substituted heterocycle is heterocycle as defined above having one or more substituents independently selected from C₁-C₄ alkyl, —O—C₁-C₄ alkyl, —S—CH₃, —N(CH₃)₂, thiazolyl, and oxazolyl;

X is O or S; and

t is an integer from 0 to 2.

In still another aspect of the preceding embodiment, R¹ is

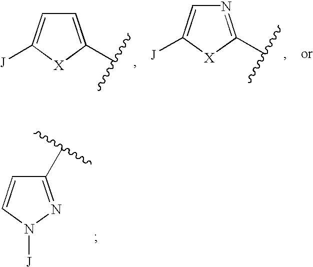

wherein J is

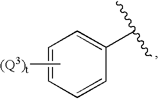

heterocycle, or substituted heterocycle;

each Q³ is independently hydrogen, halogen, cyano, C₁-C₄ alkyl, or —O—C₁-C₄ alkyl;

heterocycle is

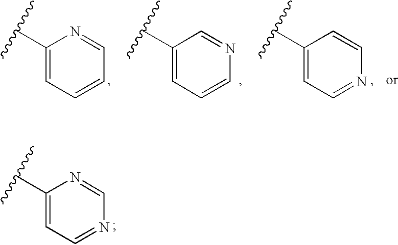

substituted heterocycle is heterocycle as defined above having one or more substituents independently selected from C₁-C₄ alkyl, —O—C₁-C₄ alkyl, —S—CH₃, —N(CH₃)₂, thiazolyl, and oxazolyl; and

X is O or S; and

t is an integer from 0 to 2.

In still another aspect of the preceding embodiment, R¹ is

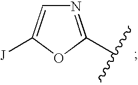

wherein J is

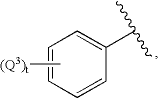

heterocycle, or substituted heterocycle;

each Q³ is independently hydrogen, halogen, cyano, C₁-C₄ alkyl, or —O—C₁-C₄ alkyl;

heterocycle is

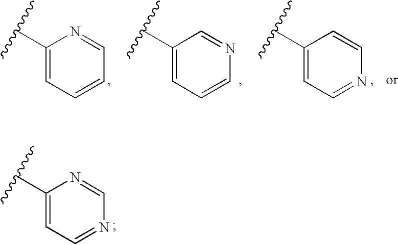

substituted heterocycle is heterocycle as defined above having one or more substituents independently selected from C₁-C₄ alkyl, —O—C₁-C₄ alkyl, —S—CH₃, —N(CH₃)₂, thiazolyl, and oxazolyl; and

t is an integer equal to zero, 1 or 2.

Another embodiment of the present invention is a compound of Formula III, wherein R² and R³ are each independently hydrogen or C₁-C₄ alkyl;

and all other variables are as originally defined or as defined in any of the preceding embodiments or aspects;

or a salt thereof.

An aspect of the preceding embodiment is a compound of Formula III, wherein R² and R³ are either both —H or both methyl. Another aspect of the preceding embodiment is a compound of Formula III, wherein R² and R³ are both methyl.

Another embodiment of the present invention is a compound of Formula III, wherein R⁴ and R⁵ are each independently —C₁-C₄ alkyl which is optionally substituted with one or more substituents each of which is independently:

(a) halogen,

(b) —O—C₁-C₄ alkyl,

(c) —O—C₁-C₄ haloalkyl,

(d) —C₁-C₄ alkyl-OR^{e}, or

(e) —N(R^{e})₂;

and all other variables are as originally defined or as defined in any of the preceding embodiments or aspects;

or a salt thereof.

An aspect of the preceding embodiment is a compound of Formula III, wherein R⁴ and R⁵ are both methyl.

Another embodiment of the present invention is a compound of Formula III, wherein R⁶ is C₁-C₆ alkyl optionally substituted with one or more halogens each of which is independently fluoro, chloro, or bromo;

and all other variables are as originally defined or as defined in any of the preceding embodiments or aspects;

or a salt thereof.

Yet another embodiment of the present invention is a compound of Formula III, wherein R⁶ is C₁-C₄ alkyl or C₁-C₄ fluoroalkyl;

and all other variables are as originally defined or as defined in any of the preceding embodiments or aspects;

or a salt thereof.

Still another embodiment of the present invention is a compound of Formula III, wherein R⁶ is

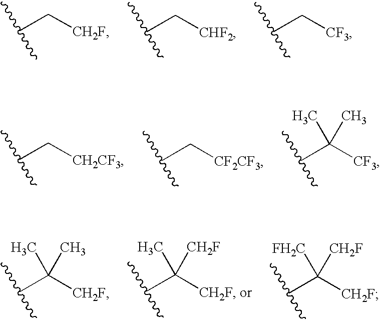

and all other variables are as originally defined or as defined in any of the preceding embodiments or aspects;

or a salt thereof.

An aspect of the preceding embodiment is a compound of Formula III, wherein R⁶ is

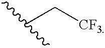

Another embodiment of the present invention is a compound of Formula (IIIa):

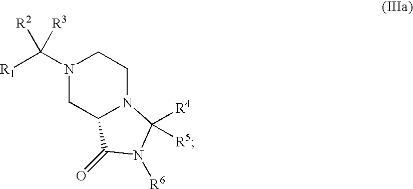

or a salt thereof; wherein each variable is independently as originally defined above or as defined in any of the preceding embodiments or aspects.

Another embodiment of the present invention is a compound of Formula (III-A1):

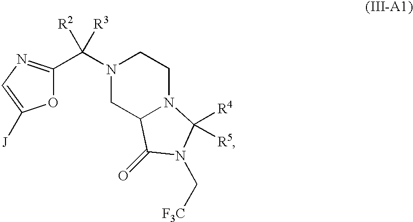

wherein

J is

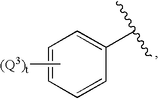

heterocycle, or substituted heterocycle;

heterocycle is

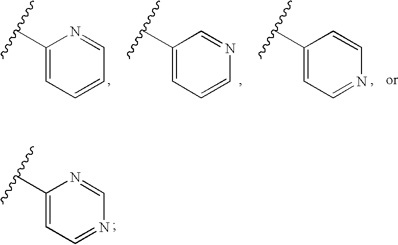

substituted heterocycle is heterocycle as defined above having one or more substituents independently selected from C₁-C₄ alkyl, —O—C₁-C₄ alkyl, —S—CH₃, —N(CH₃)₂, thiazolyl, and oxazolyl;

each Q³ is independently hydrogen, halogen, cyano, C₁-C₄ alkyl, or —O—C₁-C₄ alkyl;

t is an integer equal to zero, 1 or 2;

R² and R³ are each independently hydrogen or C₁-C₄ alkyl; and

R⁴ and R⁵ are each independently —C₁-C₄ alkyl which is optionally substituted with one or more substituents each of which is independently:

(a) halogen,

(b) —O—C₁-C₄ alkyl,

(c) —O—C₁-C₄ haloalkyl,

(d) —C₁-C₄ alkyl-OR^{e}, or

(e) —N(R^{e})₂;

each R^{e} is independently a —C₁-C₄ alkyl;

or a salt thereof.

An aspect of the preceding embodiment is a compound of Formula (III-A1a):

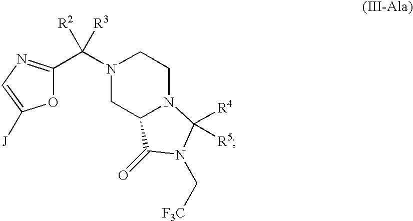

or a salt thereof; wherein J, R², R³, R⁴ and R⁵ are each as defined in the preceding paragraph.

Still another embodiment of the present invention is a compound of Formula (III-A2):

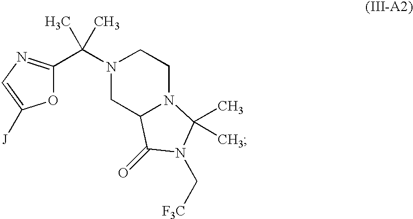

wherein J is

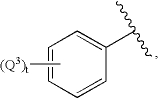

heterocycle, or substituted heterocycle;

each Q³ is independently hydrogen, halogen, cyano, C₁-C₄ alkyl, or —O—C₁-C₄ alkyl;

heterocycle is

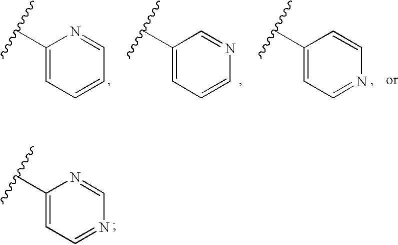

substituted heterocycle is heterocycle as defined above having one or more substituents independently selected from C₁-C₄ alkyl, —O—C₁-C₄ alkyl, —S—CH₃, —N(CH₃)₂, thiazolyl, and oxazolyl; and

t is an integer from 0 to 2;

or a salt thereof.

An aspect of the preceding embodiment is a compound of Formula (III-A2a):

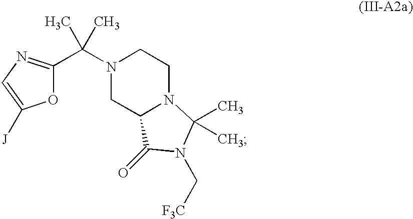

or a salt thereof; wherein J is as defined in the preceding paragraph.

A further embodiment of the present invention is Compound 13:

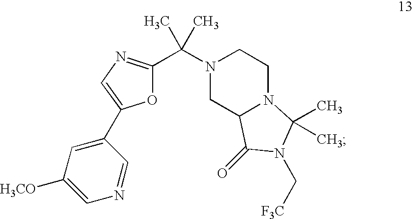

or a salt thereof.

An aspect of the preceding embodiment is Compound 13a:

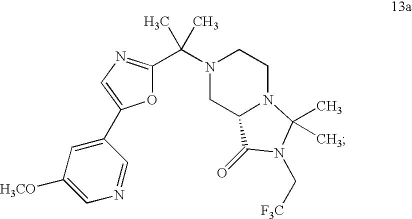

or a salt thereof.

The present invention also includes a process for preparing a compound of Formula (III), which comprises:

(C) coupling an iminium salt of Formula I:

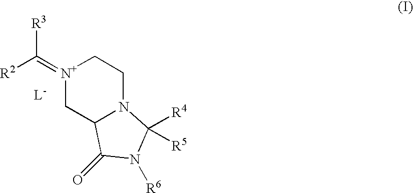

with a metallated derivative of a compound of Formula (II):

R¹—H (II),

in solvent to obtain Compound III;

wherein L⁻0 is a counterion; and R¹, R², R³, R⁴, R⁵, and R⁶ are each independently as originally defined above or as defined in any one of the foregoing embodiments or aspects.

In an embodiment of the process, L⁻ is selected from the group consisting of:

(1) halide,

(2) cyanide,

(3) BF₄⁻,

(4) (C₆F₅)₄B⁻,

(5) MF₆⁻, wherein M is P, As, or Sb,

(6) ClO₄⁻,

(7) benzotriazolyl anion,

(8) aryl-SO₃⁻, wherein the aryl is optionally substituted with one or more substituents each of which is independently halo, C₁-C₁₀ alkyl, or C₁-C₁₀ haloalkyl,

(9) C₁-C₆ alkyl-SO₃⁻ wherein the alkyl is optionally substituted with one or more halogens, and

(10) trihaloacetate anion.

In another embodiment of the process, L⁻ is selected from the group consisting of fluoride, chloride, cyanide, BF₄⁻, (C₆F₅)₄B⁻, PF₆⁻, ClO₄⁻, benzotriazolyl anion, OTf⁻, CF₃CF₂SO₃⁻, C₆F₅SO₃⁻, OTs⁻, and CF₃CO₂⁻.

In still another embodiment of the process, L⁻ is a weakly nucleophilic or non-nucleophilic anion. Stated alternatively, L⁻ in this embodiment is a very weak base and when L is attached to carbon, L can be readily displaced as L⁻ by a variety of nucleophiles. In an aspect of this embodiment, L⁻ is selected from the group consisting of fluoride, chloride, BF₄⁻, (C₆F₅)₄B⁻, PF₆⁻, AsF₆⁻, SbF₆⁻, ClO₄⁻, benzotriazolyl anion, OTf⁻, CF₃CF₂SO₃⁻, C₆F₅SO₃⁻, OTs⁻, and CF₃CO₂⁻.

The iminium salt of Formula (I) may exist, in whole or in part, as the the corresponding compound of Formula (I-A):

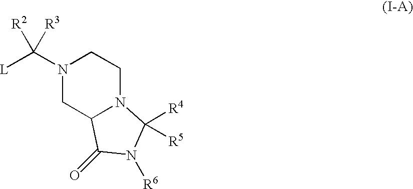

Whether or not a covalent compound or a salt is the sole or preferred form at ambient temperature and pressure depends to a large extent upon the nature of L⁻. When L⁻ is a relatively strong base such as cyanide, for example, the substance will typically exist, at least in part, in the I-A form. On the other hand, when L⁻ is a relatively weak base such as OTf⁻, the substance is more typically isolated in the salt form. In any event, it is to be understood that, unless expressly stated to the contrary, a reference herein to a salt of Formula (I) or to a covalent compound of Formula (I-A) means a reference to the iminium salt I, the corresponding compound of Formula (I-A), or mixtures thereof.

When L⁻ is CN—, it is preferred to conduct the coupling reaction of Step C in the presence of a Lewis acid. Suitable Lewis acids include those selected from the group consisting of (R\hat{ })ₚAl(Y)₃₋ₚ, BY₃, TiY₄, FeY₃, SnY₄, Ti(OR\hat{ })₄, and (R\hat{ })₃SiOTf; wherein each R\hat{ } is independently a C₁-C₄ alkyl; each Y is independently a halogen, and p is an integer equal to zero, 1, 2, or 3. In one embodiment, the Lewis acid is selected from the group consisting of AlCl₃, BF₃, TiCl₄, FeCl₃, SnCl₄, Ti(OR\hat{ })₄, and TBSOTf. In an aspect of the preceding embodiment, the Lewis acid is TBSOTf. The Lewis acid is suitably employed in an amount of at least about 1 equivalent per equivalent of Compound I, and is typically employed in an amount of from about 1 to about 2 equivalents (e.g., from about 1 to about 1.5 equivalents) per equivalent of I.

The solvent employed in the coupling reaction of Step C can be any organic compound which under the reaction conditions employed is in the liquid phase, is chemically inert, and will dissolve, suspend, and/or disperse the reactants. Suitable solvents include C₃-C₁₀ linear and branched alkanes, C₁-C₁₀ linear and branched halogenated alkanes, C₅-C₁₀ cycloalkanes, C₆-C₁₄ aromatic hydrocarbons, dialkyl ethers wherein each alkyl is independently a C₁-C₆ alkyl, C₁-C₆ linear and branched alkanes substituted with two —O—C₁-C₆ alkyl groups (which are the same or different), C₄-C₈ cyclic ethers and diethers, and C₆-C₈ aromatic ethers. In one embodiment of the process, the solvent is selected from the group consisting of C₁-C₁₀ linear and branched halogenated alkanes, dialkyl ethers wherein each alkyl is independently a C₁-C₆ alkyl, C₁-C₆ linear and branched alkanes substituted with two —O—C₁-C₆ alkyl groups (which are the same or different), and C₄-C₈ cyclic ethers and diethers. In an aspect of this embodiment, the solvent is a dialkyl ether, wherein each alkyl is independently a C₁-C₄ alkyl, or a C₄-C₈ cyclic ether or diether. Exemplary solvents include pentane, hexane, carbon tetrachloride, chloroform, methylene chloride, 1,2-dichloroethane, 1,1,2-trichloroethane, 1,1,2,2-tetrachloroethane, cyclohexane, toluene, o- and m- and p-xylene, ethylbenzene, ethyl ether, MTBE, THF, dioxane, 1,2-dimethoxyethane, anisole, and phenetole. Additional suitable solvents include ureas (e.g., DMEU or DMPU) and HMPA.

Coupling Step C is suitably conducted at a temperature in the range of from about −80 to about 20° C., and typically at a temperature in the range of from about −60 to about 10° C. In one embodiment, the temperature is in the range of from about −10 to about 0° C.

The metallated derivative of R¹—H can be employed in the coupling step in any proportion with respect to iminium salt I which will result in the formation of at least some of Compound III. Of course, the reactants will typically be employed in a proportion which, under the selected reaction conditions (e.g., temperature, degree of agitation), will permit the reaction to proceed to completion (i.e., complete or nearly complete conversion of the iminium salt) within a reasonable time. In one embodiment, the metallated derivative of Compound II is present in an amount in the range of from about 0.5 to about 5 equivalents (e.g., from about 0.9 to about 3 equivalents) per equivalent of Compound I. In another embodiment, the metallated derivative of Compound II is present in an amount in the range of from about 1 to about 2 equivalents per equivalent of Compound I. In still another embodiment, the metallated derivative of Compound II is present in an amount in the range of from about 1 to about 1.5 equivalents (e.g., from about 1 to about 1.1 equivalents) per equivalent of Compound I.

In a suitable procedure for conducting the coupling reaction of Step C, a solution of the metallated derivative of II is added to a solution of the iminium salt I. The solutions are mixed at low temperatures (e.g., less than about 5° C.), and the resulting reaction mixture is maintained at low temperature until the reaction is complete. The coupled product III can then be recovered via conventional means.

As used herein, the term “metallated derivative” means a derivative which contains a carbon-metal bond, which bond can range in character from covalent to ionic. Metallation (i.e., the formation of the carbon-metal bond) can be accomplished by treating the starting compound (i.e., R¹—H) with a metal-containing base having sufficient strength to cause deprotonation. Suitable metal-containing deprotonating agents (which may alternatively be referred to as metallating agents) include the alkali metals per se, alkaline earth metal halides, Group 2b transition metal halides, alkali metal salts and alkaline earth metal salts of di-C₁-C₆ alkylamines and C₄-C₈ cyclic secondary amines, alkali metal salts and alkaline earth metal salts of bis(tri-C₁-C₄ alkylsilyl)amines, alkali metal hydrides, alkali metal amides, C₁-C₆ alkyllithiums, C₆-C₁₀ aryllithiums, C₁-C₆ alkylmagnesium halides, C₆-C₁₀ arylmagnesium halides, and C₁-C₆ alkoxides of alkali and alkaline earth metals.

Exemplary deprotonating agents include lithium metal, methyllithium, n-butyllithium, tert-butyllithium, sec-butyllithium, phenyllithium, phenyl sodium, phenyl potassium, lithium amide, sodium amide, potassium amide, lithium tetramethylpiperidide, lithium diisopropylamide, lithium diethylamide, lithium dicyclohexylamide, sodium hexamethyldisilazide, lithium hexamethyldisilazide, sodium hydride, potassium hydride, sodium methoxide, potassium methoxide, sodium ethoxide, potassium ethoxide, ethylmagnesium chloride, isopropylmagnesium chloride, phenylmagnesium chloride, ethylmagnesium bromide, isopropylmagnesium bromide, and phenylmagnesium bromide.

In one embodiment, the deprotonating agent is a strong organometallic base. In an aspect of this embodiment, the deprotonating agent is selected from the group consisting of C₁-C₆ alkyllithiums, C₆-C₁₀ aryllithiums, and C₁-C₆ alkylmagnesium halides. In another aspect of this embodiment, the organometallic base has a pKa of about 25 or more.

In the case of R¹=alkyl or cycloalkyl, metallation can alternatively be accomplished by treating a bromide or chloride derivative of the starting compound (i.e., R¹—Cl or R¹—Br) with an active metal per se, especially Li. The metallated (cyclo)alkyl derivative is formed by treating its corresponding bromide or chloride with the active metal in a suitable solvent (e.g., anhydrous ether) at low temperature (e.g., from about −80 to about 0° C.).

When R¹ is alkenyl, alkynyl, aryl, or heteroaryl, the agent is typically one of the deprotonating agents set forth above other than alkali metals per se. Metallation is typically effected by contacting about one equivalent of the deprotonating agent in a suitable solvent with the starting compound (optionally also in the same or a different solvent) under conditions and for a time sufficient to obtain the metallated derivative. The choice of solvent(s), reactions conditions, and reaction time will vary with the substrate and choice of deprotonating agent. The solvent(s) of course must be chemically inert and must be able to dissolve (or suspend or disperse) the reactants sufficiently to permit intimate contact between the reactants. Suitable solvents include ethers and hydrocarbons, including C₃-C₁₀ linear and branched alkanes, C₅-C₁₀ cycloalkanes, C₆-C₁₄ aromatic hydrocarbons, di-C₁-C₆ alkyl ethers, C₁-C₆ linear and branched alkanes substituted with two —O—C₁-C₆ alkyl groups (which are the same or different), and C₄-C₈ cyclic ethers and diethers. Exemplary solvents include diethyl ether, THF, hexane, and benzene. The reaction is generally conducted at low temperature. The reaction temperature is suitably below about 10° C. and is typically in a range of from about −80 to about 5° C. The reaction time can vary widely depending upon the choice of substrate, deprotonating agent, solvent, and temperature, but is typically about 24 hours or less (e.g., about 12 hours or less). The reaction is generally conducted under dry conditions and under an atmosphere of inert gas (e.g., nitrogen).

For the purposes of this invention a metallated derivative includes derivatives which contain a carbon-silicon bond. Accordingly, the metallated derivative can also be a silylated derivative of R¹—H. Suitable silylated derivatives include —SiR₃ derivatives wherein each R is independently C₁-C₆ alkyl or aryl (e.g., phenyl). In one embodiment, the silyl derivative is a tri-C₁-C₆ alkyl silyl derivative, such as trimethylsilyl (TMS), t-butyldimethylsilyl (TBS), or tri-isopropylsilyl (TIPS). The silylated derivatives of R¹—H can be prepared by treating the starting compound with a deprotonating agent such as those described above (e.g., an alkyllithium such as n-butyllithium) and then treating the product with a tri-alkyl silyl halide (e.g., TMSCl, TBSCl, or TIPSCl) or with a sulfonate (e.g., TBSOTf or TIPSOTf). Of particular use in the practice of the present invention are silyl derivatives of R¹—H wherein R¹ is aryl or heteroaryl.

In one embodiment, the metallated derivative is a zinc or copper derivative of R¹—H. Suitable derivatives can be obtained by reacting a zinc or copper salt (e.g., halides) in an inert solvent (e.g., THF or diethyl ether) with the corresponding lithiated derivative (e.g., prepared by treating R¹—H with an alkyllithium such as those described above) or magnesiated derivative (e.g., prepared by treating R¹—H with an alkylmagnesium halide such as those described above). Further description of this method and of other methods for preparing Zn and Cu metallated derivatives is presented in Comprehensive Organometallic Chemistry, edited by G. Wilkinson, Vol. 2, Pergamon Press, 1982, pp. 715-718 and 832-833. Bimetallated derivatives can also be employed, such as the ZnCu metallated oxazoles described in Harn et al., Tet. Letters 1995, 36: 9453-9456.

In the case where R¹ is heteroaryl, the metallation will typically occur alpha to a heteroatom due to the inductive effect of the heteroatom, although experimental conditions such as the identity of the base and solvents, order of reagent addition, and temperature of addition can be modified by one skilled in the art to achieve the desired metallation position. Alternatively, the position of metallation can be controlled by use of a halogenated heteroaryl, wherein the halogen is located on the position of the heteroaryl ring where metallation is desired (see, e.g., Joule et al., Heterocyclic Chemistry, 3^{rd} edition, 1995, p. 33). Halogenated heteroaryls are available commercially or can be prepared by well-known synthetic methods.

Further description of methods for metallating organic compounds can be found in Gilman and Morton, “The Metallation Reaction with Organolithium Compounds”, Chapter 6 in Organic Reactions, 8, 258-304 (1954); Gschwend and Rodriguez, “Heteroatom-Facilitated Lithiations”, Chapter 1 in Organic Reactions, 26, 1-3600 (1979); Wakefield, Organolithium Methods, Academic Press, London, 1988; Wakefield, Organomagnesium Methods in Organic Synthesis, Academic Press, London, 1995; and Joule et al., Heterocyclic Chemistry, 3^{rd} edition, 1995, p. 30-37. The procedures described in these references can be used, or can be adapted for use without undue experimentation, by a person of ordinary skill in the art to prepare metallated derivatives of R¹—H.

An embodiment of the process of the invention directed to Step C is a process for preparing a compound of Formula (IIIa):

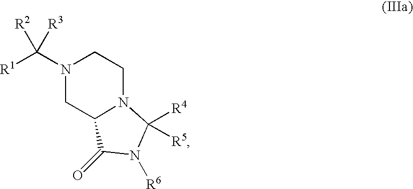

which comprises:

(C) coupling an iminium salt of Formula Ia:

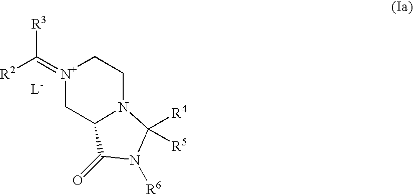

with a metallated derivative of a compound of Formula (II):

R¹—H (II),

in solvent to obtain Compound IIIa;

wherein L⁻, R¹, R², R³, R⁴, R⁵, and R⁶ are each independently as originally defined above or as defined in any one of the foregoing embodiments or aspects. Choice of solvents, reaction conditions, and relative amounts of reactants and reagents are as described above.

Still another embodiment of the present invention directed to Step C is a process for preparing a compound of Formula (III-A2):

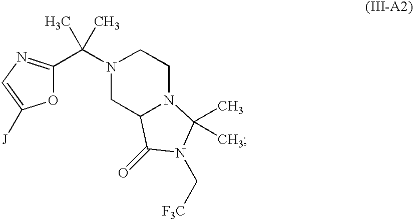

which comprises:

(C) coupling an iminium salt of Formula (I-A):

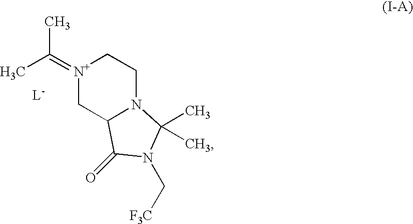

with a metallated derivative of a compound of Formula (II-A):

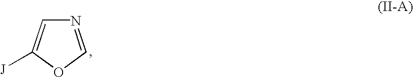

in solvent to obtain compound III-A2.

wherein J and L are each independently as originally defined above or as defined in any embodiments or aspects as set forth above. Choice of solvents, reaction conditions, and relative amounts of reactants and reagents are as described above.

In an aspect of the process set forth in the preceding embodiment, the iminium salt I-A is an iminium salt of Formula (I-Aa):

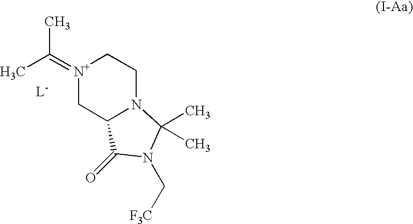

and the compound obtained from Step C is a compound of Formula (III-A2a):

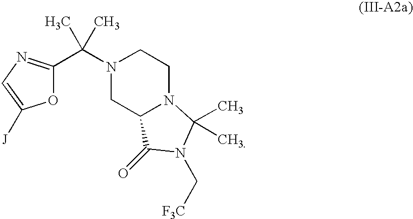

The compounds embraced by Formula (II) in the above-described processes of the invention (i.e., compounds of formula R¹—H) include certain alkanes, alkenes, cycloalkanes, aromatics and heteroaromatics. Many of these compounds are available commercially, but otherwise the compounds can be prepared by methods known in the art or by routine variations thereof.

In one embodiment, Compound II is an oxazole. The oxazoles can be prepared as described in Turchi et al., Chem. Rev. 1975, 75 (4): 389-437 or in Rodd's Chemistry of Carbon Compounds, edited by S. Coffey and M. F. Ansell, Vol. IV, Part C, Elsevier, 1986, pp. 303-346.

In another embodiment, Compound II is an oxazole of formula (II-A) as defined above. Oxazole II-A can be prepared by treating an aldehyde of formula J-CHO with p-tosylmethylisocyanide (TosMic) and a base. The reaction can be carried out in polar organic solvents such as alcohols or ethers. Suitable alcohols include C₁-C₆ alkyl alcohols. Suitable ethers include dialkyl ethers wherein each alkyl is independently a C₁-C₆ alkyl, C₁-C₆ linear and branched alkanes substituted with two —O—C₁-C₆ alkyl groups (which are the same or different), and C₄-C₈ cyclic ethers and diethers. Suitable bases include alkali metal carbonates and bicarbonates (e.g., Na₂CO₃, K₂CO₃, and KHCO₃) and alkali metal alkoxides (e.g., C₁-C₆ alkoxides of sodium and potassium). The aldehyde and TosMic are typically reacted together in equimolar amounts in the presence of the base at a temperature in the range from about −80 to about 25° C. Further description of the preparation of 5-substituted oxazoles from aldehydes and TosMic can be found in van Leusen et al., Tet. Letters 1972, pp. 2369-2372.

The present invention includes 5-substituted oxazoles of Formula II-A and salts thereof (e.g., inorganic and organic acid addition salts), wherein J is pyridyl or pyrimidinyl, either of which is optionally substituted with from 1 to 3 substituents each of which is independently C₁-C₄ alkyl, —O—C₁-C₄ alkyl, —S—CH₃, —N(CH₃)₂, thiazolyl, or oxazolyl. In one embodiment, J is pyridyl substituted with from 1 to 3 substituents each of which is independently C₁-C₄ alkyl or —O—C₁-C₄ alkyl. An aspect of this embodiment is a 5-substituted oxazole of Formula II-A in which J is pyridyl substituted with 1 or 2-O—C₁-C₄ alkyl groups. Another aspect of this embodiment is Compound 4:

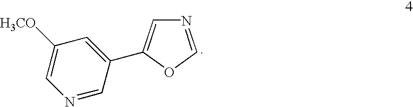

The present invention also includes a process which comprises Step C as heretofore described, and which further comprises:

(D) treating Compound III with acid to obtain a compound of Formula (IV):

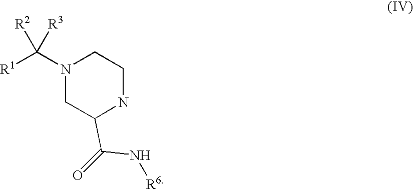

Step D is an acid deprotection step which affords Compound IV, wherein R¹, R², R³ and R⁶ are as originally defined above (see the discussion of Compound III) or as defined in any of the embodiments set forth above. In Step D, Compound III dissolved in a suitable solvent is brought into contact with the acid. Suitable solvents include polar organic solvents which are chemically inert under the conditions employed in Step D, such as ethers, nitriles, and esters. In one embodiment, the solvent is a dialkyl ether wherein each alkyl is independently a C₁-C₆ alkyl, C₁-C₆ linear or branched alkane substituted with two —O—C₁-C₆ alkyls (which are the same or different), a C₄-C₈ cyclic ether and diether, C₂-C₆ aliphatic nitriles, and C₁-C₆ alkyl esters of C₁-C₆ alkylcarboxylic acids. Exemplary solvents include diethyl ether, THF, acetonitrile, propionitrile, methyl acetate, ethyl acetate, and isopropyl acetate.

Suitable acids include HCl, HBr, sulfuric acid, tetrafluoroboric acid, phosphoric acid, nitric acid, and perchloric acid or an organic acid selected from the group consisting of R^{u}—SO₃H, R^{v}—SO₃H, and R^{v}—CO₂H; wherein R^{u} is aryl optionally substituted with from 1 to 5 substituents each of which is independently halo, C₁-C₈ alkyl, or C₁-C₈ haloalkyl, and R^{v} is C₁-C₆ alkyl optionally substituted with from 1 to 7 halogens. Exemplary organic acids include trifluoroacetic acid, naphthalenesulfonic acid, benzenesulfonic acid, methanesulfonic acid, toluenesulfonic acid, and triflic acid. The acids are employed in a protic medium such as water or an alcohol. The acid in solution or admixture with water or an alcohol (e.g., methanol or ethanol) is typically charged into a solution of Compound III. The acid treatment can be conducted at a temperature in the range of from about −50 to about 150° C., and is typically conducted at a temperature in the range of from about −20 to about 80° C. (e.g, from about −10 to about 30° C.). In one embodiment, the temperature is in the range of from about 20 to about 30° C. The acid is suitably employed in an amount in the range of from about 0.1 to about 5 equivalents per equivalent of Compound III. In one embodiment, a catalytic amount of the acid is employed, such as an amount of from about 0.1 to about 0.5 equivalents per equivalent of Compound III. Stoichiometric or greater amounts of acid can be employed, particularly if it is desired to facilitate crystallization of the product. In a suitable procedure, a solution of the acid is added slowly (e.g., dropwise) to a solution of Compound III while maintaining the solution at a relatively low temperature, in order to avoid a rapid accumulation of heat. Once the reaction is complete or the desired degree of conversion has been obtained, the reaction mixture can be quenched with base and product IV recovered by conventional means.

In an aspect of the process comprising Steps C and D, Compound III is Compound IIIa as heretofore defined, and the compound resulting from treating Compound IIIa with acid is a compound of Formula (IVa):

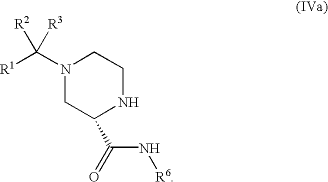

The present invention also includes a process for preparing Compound 13:

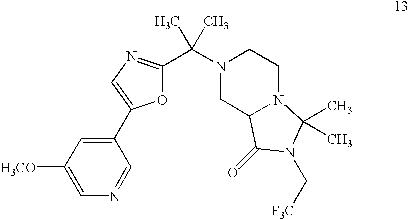

which comprises:

(cc) coupling Compound 10:

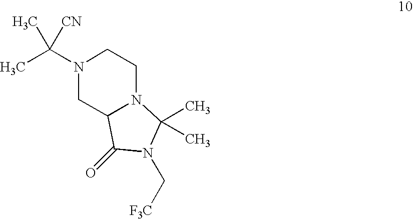

in the presence of a Lewis acid, or coupling compound I-A:

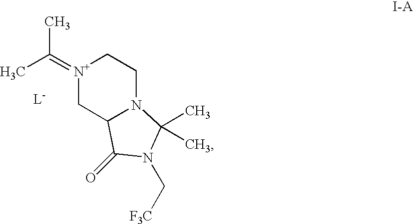

with a metallated derivative of Compound 4:

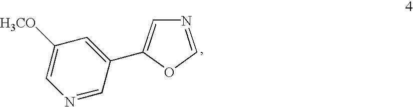

in solvent to obtain 13; wherein L⁻ is a non-nucleophilic counterion.

Embodiments of this process include the process as just described additionally incorporating one or more of the following features:

the metallated derivative is prepared by treating 4 with a strong organometallic base;

the metallated derivative is prepared by treating 4 with a deprotonating agent selected from the group consisting of C₁-C₆ alkyllithiums, C₆-C₁₀ aryllithiums, and C₁-C₆ alkylmagnesium halides;

the metallated derivative is prepared by treating 4 with a C₁-C₆ alkylmagnesium halide (e.g., isopropylmagneisum chloride or bromide); deprotonating agent;

the Lewis acid is selected from the group consisting of AlCl₃, BF₃, TiI₄, FeCl₃, SnCl₄, Ti(OR^{b})₄, and TBSOTf;

the Lewis acid is TBSOTf;

L⁻ is selected from the group consisting of (1) halide, (2) BF₄⁻, (3) (C₆F₅)₄B⁻, (4) MF₆⁻, wherein M is P, As, or Sb, (5) ClO₄⁻, (6) benzotriazolyl anion, (7) aryl-SO₃⁻, wherein the aryl is optionally substituted with one or more substituents each of which is independently halo, C₁-C₁₀ alkyl, or C₁-C₁₀ haloalkyl, (8) C₁-C₆ alkyl-SO₃⁻ wherein the alkyl is optionally substituted with one or more halogens, and (9) trihaloacetate anion;

L⁻ is selected from the group consisting of fluoride, chloride, BF₄⁻, (C₆F₅)₄B⁻, PF₆⁻, AsF₆⁻, SbF₆⁻, ClO₄⁻, benzotriazolyl anion, OTf⁻, CF₃CF₂SO₃⁻, C₆F₅SO₃⁻, OTs⁻, and CF₃CO₂⁻;

L⁻ is OTf⁻;

the solvent in Step (cc) is an ether (e.g., dialkyl ether wherein each alkyl is independently a C₁-C₄ alkyl or a C₄-C₈ cyclic ether or diether (e.g., THF));

the coupling reaction is conducted at a temperature in the range of from about −80 to about 20° C. (e.g., from about −10 to about 0° C.); or

the metallated derivative of 4 is present in an amount in the range of from about 1 to about 2 equivalents (e.g., from about 1 to about 1.5 equivalents or from about 1 to about 1.1 equivalents) per equivalent of 10 or per equivalent of I-A.

An aspect of the process for preparing Compound 13 is a process for preparing Compound 13a which comprises:

(cc) coupling Compound 10a:

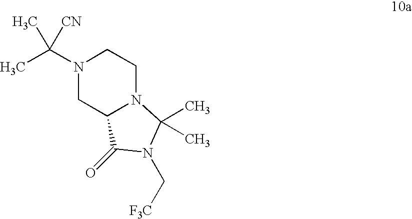

in the presence of a Lewis acid, or coupling compound I-Aa (defined above), with a metallated derivative of Compound 4 in solvent to obtain 13a.

The present invention also includes a process for preparing Compound 15:

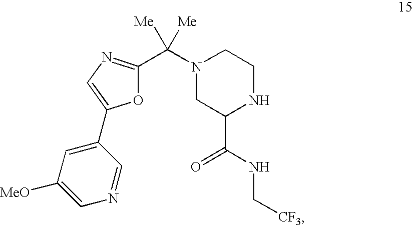

which comprises Step (cc) as originally set forth above and further comprises:

(dd) treating Compound 13 with acid to obtain Compound 15.

Embodiments of this process include the process as just described incorporating one or more of the features set forth above for Step (cc) and/or incorporating one or more of the following features:

the acid is an aqeuous or alcoholic solution of HCl;

the acid is an aqueous or alcoholic solution of naphthalenesulfonic acid (e.g., 2-naphthalenesulfonic acid);

the acid is employed in a stoichiometric or a catalytic amount;

the acid is 2-naphthalenesulfonic acid (e.g., as an aqueous solution) employed in an amount of about 3 equivalents per equivalent of Compound 13, and the process optionally further comprises isolating a crystalline tris 2-NSA salt of Compound 15;

the treatment step (dd) is conducted at a temperature of from about −20 to about 80° C.

An aspect of the process for preparing Compound 15 is a process for preparing Compound 15a:

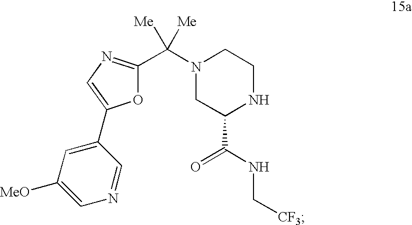

which comprises Step (cc) as set forth above for preparing Compound 13a from Compound 10a and further comprises:

(dd) treating Compound 13a with acid to obtain Compound 15a.

The present invention also includes a process for preparing a compound of Formula (VI):

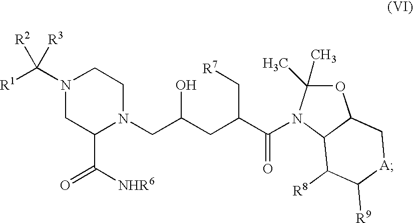

which comprises Steps C and D as heretofore described and which further comprises:

(E) reacting piperazine carboxamide IV with an epoxide of Formula (V):

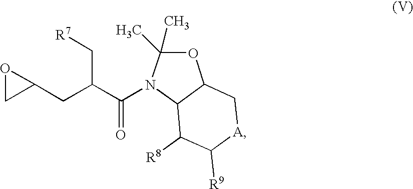

to obtain a compound of Formula (VI); wherein

R¹, R², R³ and R⁶ are as originally defined above (see discussion of Compound III) or as defined in any of the embodiments or aspects set forth above;

A is absent, CH₂, O, or S;

R⁷ is C₁-C₆ alkyl, C₃-C₆ cycloalkyl, aryl, or heteroaryl; wherein the alkyl or cycloalkyl is optionally substituted with one or more substituents each of which is independently halogen, hydroxy, —C₁-C₆ haloalkyl, —O—C₁-C₆ alkyl, or —O—C₁-C₆ haloalkyl; and wherein aryl or heteroaryl is optionally substituted with one or more substituents each of which is independently halogen, hydroxy, —C₁-C₆ haloalkyl, —O—C₁-C₆ alkyl, —O—C₁-C₆ haloalkyl, C₂-C₆ alkenyl, or C₂-C₆ alkynyl; and

R⁸ and R⁹ are each independently —H, —C₁-C₆ alkyl, —C₁-C₆ haloalkyl, —C₃-C₆ cycloalkyl, or aryl, wherein the aryl is optionally substituted with one or more substituents each of which is independently halogen, —OH, —C₁-C₆ alkyl, —C₁-C₆ haloalkyl, —O—C₁-C₆ alkyl, or —O—C₁-C₆ haloalkyl; or alternatively

R⁸ and R⁹ together with the carbons to which each is attached form a fused benzene ring which is optionally substituted with one or more substituents each of which is independently halogen, —OH, —C₁-C₆ alkyl, —C₁-C₆ haloalkyl, —O—C₁-C₆ alkyl, or —O—C₁-C₆ haloalkyl.

Steps C and D of this process have already been described in detail above. It is understood that embodiments of this process include the Steps C, D and E as originally described above, incorporating one or more embodiments, aspects, or features of either or both of Steps C and D as set forth above and/or incorporating one or more embodiments, aspects or features of Step E as set forth below.

In an embodiment of the process, A in Compounds V and VI is absent, CH₂, or O. In another embodiment, A is absent or O. It is understood that “A is absent” means that a ring is formed via a direct single bond between the atoms that would otherwise have been directly attached to A. For example, when A is absent, Compound V has the following structure:

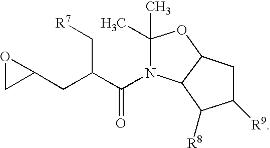

In another embodiment of the process, R⁷ in Compounds V and VI is C₁-C₆ alkyl, C₃-C₆ cycloalkyl, phenyl, or heteroaryl, wherein heteroaryl is selected from pyridyl, pyrazinyl, pyrimidinyl, thiophenyl, thiazolyl, pyridofuranyl, pyrimidofuranyl, pyridothienyl, pyridazothienyl, pyridooxazolyl, pyridazooxazolyl, pyrimidooxazolyl, pyridothiazolyl, and pyridazothiazolyl; and wherein phenyl or heteroaryl is optionally substituted with one or more substituents each of which is independently halogen, hydroxy, C₁-C₆ alkyl, C₁-C₆ fluoroalkyl, —O—C₁-C₆ alkyl, or —O—C₁-C₆ fluoroalkyl.

In another embodiment, R⁷ in Compounds V and VI is

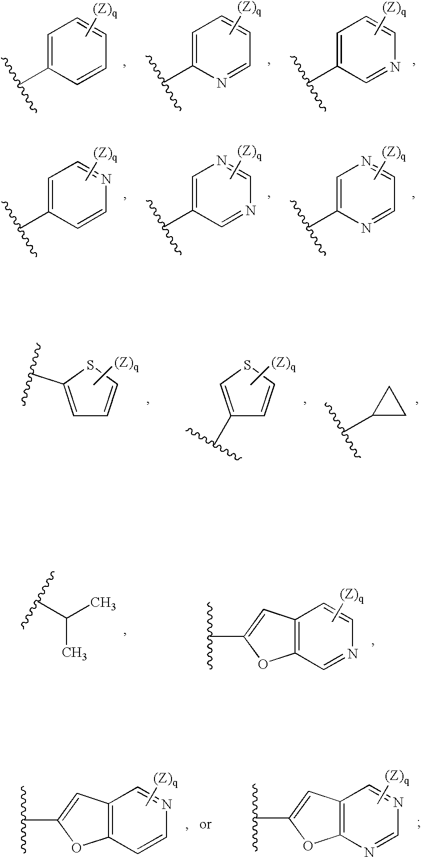

each Z is independently hydrogen, halogen, cyano, C₁-C₆ alkyl, or —O—C₁-C₆ alkyl; and

q is an integer from 0 to 2.

In still another embodiment, R⁷ in Compounds V and VI is

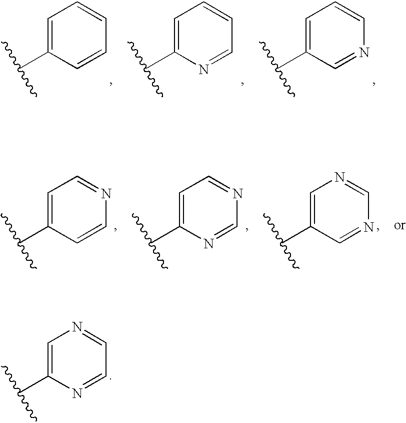

In still another embodiment, R⁷ in Compounds V and VI is

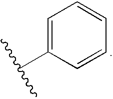

In another embodiment of the process, R⁸ and R⁹ are each independently —H, —C₁-C₄ alkyl, —C₁-C₄ haloalkyl, or phenyl, wherein the phenyl is optionally substituted with one or more substituents (e.g., substituted with from 1 to 3 substituents, or substituted with 1 or 2 substituents) each of which is independently halogen, —C₁-C₆ alkyl, —C₁-C₆ haloalkyl, —O—C₁-C₆ alkyl, or —O—C₁-C₆ haloalkyl; or alternatively R⁸ and R⁹ together with the carbons to which each is attached form a fused benzene ring which is optionally substituted with one or more substituents each of which is independently halogen, —C₁-C₆ alkyl, —C₁-C₆ haloalkyl, —O—C₁-C₆ alkyl, or —O—C₁-C₆ haloalkyl.

In another embodiment of the process, R⁸ and R⁹ are each independently —H, —C₁-C₄ alkyl, —C₁-C₄ fluoroalkyl, or phenyl; or alternatively R⁸ and R⁹ together with the carbons to which each is attached form a fused benzene ring which is optionally substituted with one or more substituents (e.g., substituted with from 1 to 3 substituents, or substituted with 1 or 2 substituents) each of which is independently halogen, —C₁-C₄ alkyl, —C₁-C₄ fluoroalkyl, —O—C₁-C₄ alkyl, or —O—C₁-C₄ fluoroalkyl.

In still another embodiment of the process, A is absent or O; and

R⁸ and R⁹ together with the carbons to which each is attached form a fused benzene ring which is optionally substituted with 1 or 2 substituents each of which is independently independently halogen, —C₁-C₄ alkyl, —C₁-C₄ fluoroalkyl, —O—C₁-C₄ alkyl, or —O—C₁-C₄ fluoroalkyl.

In another embodiment of the process, Compound V (and the corresponding moiety in Compound VI) is:

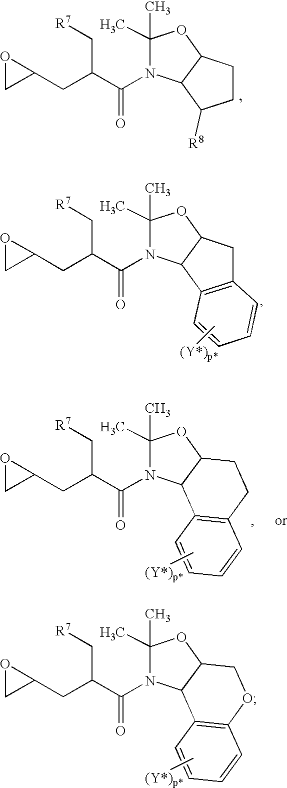

wherein R⁷ and R⁸ are each as originally defined or as defined in any of the preceding embodiments; each Y* is independently —H, halogen, —C₁-C₄ alkyl, —C₁-C₄ fluoroalkyl, or —O—C₁-C₄ alkyl; and p* is an integer equal to zero, 1 or 2.

In still another embodiment of the process, Compound V is:

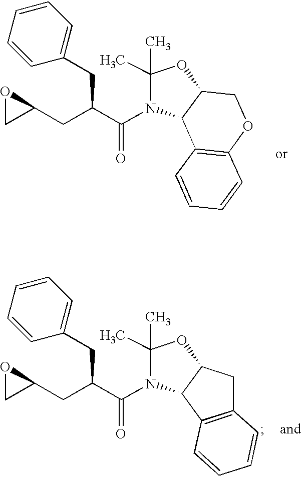

the corresponding moiety in Compound VI is:

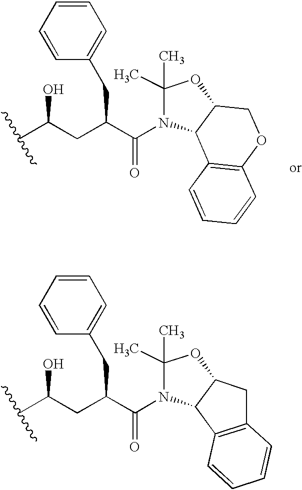

respectively.

Step E is suitably conducted in a solvent. The solvent employed in the coupling reaction can be any organic compound which under the reaction conditions employed is in the liquid phase, is chemically inert, and will dissolve, suspend, and/or disperse the reactants. Suitable solvents include hydrocarbons, ethers, alcohols, nitrites, and esters. In one embodiment, the solvent is selected from the group consisting of C₃-C₁₀ linear and branched alkanes, C₁-C₁₀ linear and branched halogenated alkanes, C₅-C₁₀ cycloalkanes, C₆-C₁₄ aromatic hydrocarbons, dialkyl ethers wherein each alkyl is independently a C₁-C₆ alkyl, C₁-C₆ linear and branched alkanes substituted with two —O—C₁-C₆ alkyl groups (which are the same or different), C₄-C₈ cyclic ethers and diethers, C₆-C₈ aromatic ethers, C₁-C₆ alkyl esters of C₁-C₆ alkylcarboxylic acids, C₁-C₁₀ alkyl alcohols, C₂-C₆ aliphatic nitrites, and C₇-C₁₀ aromatic nitriles. Exemplary solvents include carbon tetrachloride, chloroform, methylene chloride, 1,2-dichloroethane (DCE), 1,1,2-trichloroethane (TCE), 1,1,2,2-tetrachloroethane, cyclohexane, toluene, o- and m- and p-xylene, ethylbenzene, ethyl ether, MTBE, THF, dioxane, DME, anisole, phenetole, methyl acetate, ethyl acetate, ethanol, n- and iso-propanol, tert-butyl alcohol, tert-amyl alcohol, acetonitrile, propionitrile, benzonitrile, and p-tolunitrile.

In another embodiment, the solvent employed in Step E is a C₁-C₆ alkyl alcohol. In an aspect of this embodiment, the alcohol is methanol, ethanol, isopropanol, t-butyl alcohol, or t-amyl alcohol.

Step E is suitably conducted at a temperature in the range of from about room temperature up to the reflux temperature of the chosen solvent. In one embodiment, the reaction is conducted at a temperature in the range of from about 20 to about 100° C. In other embodiments, the temperature is in the range of from about 30 to about 95° C., or is in the range of from about 40 to about 95° C. (e.g., from about 45 to about 65° C.).

Piperazine carboxamide IV and epoxide V can be employed in any proportion which will result in the formation of at least some of Compound VI. Typically, however, the reactants are employed in proportions which will optimize conversion of at least one of the reactants. In one embodiment, the amount of piperazine carboxamide IV employed in Step B is at least about 0.5 equivalent per equivalent of epoxide V, and is typically in the range of from about 1 to about 5 (e.g., from about 1 to about 3) equivalents per equivalent of epoxide V. In another embodiment, piperazine carboxamide IV is employed in an amount of from about 1 to about 2 (e.g., from about 1 to about 1.5) equivalents per equivalent of epoxide V. In an aspect of the preceding embodiment, piperazine carboxamide IV is employed in an amount of from about 1 to about 1.1 equivalents per equivalent of epoxide V.

The solvent, piperazine carboxamide IV, and epoxide V can be charged to the Step E reaction vessel concurrently or sequentially in any order. In a suitable procedure, the piperazine carboxamide IV is dissolved in the chosen solvent, followed by addition of epoxide V. The mixture is then stirred at a suitable reaction temperature until the reaction is complete or, alternatively, until the desired or optimum degree of conversion is obtained.

Product VI can be recovered via conventional techniques, such as by treating a solution of VI with silica gel and/or activated carbon to remove impurities, filtering the solution, concentrating and cooling the filtrate to precipitate VI and separating VI by filtration.

Epoxides of Formula (V) for use in Step E can be prepared via the methods described in U.S. Pat. No. 5,728,840, or routine modifications thereof.

The present invention also includes a process for preparing Compound 22:

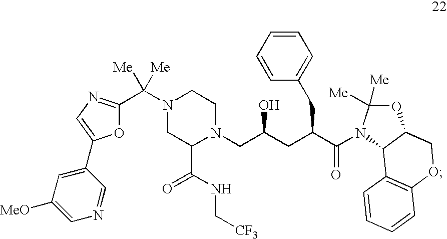

which comprises Steps (cc) and (dd) as set forth above and further comprises:

(cc) reacting piperazine carboxamide 13:

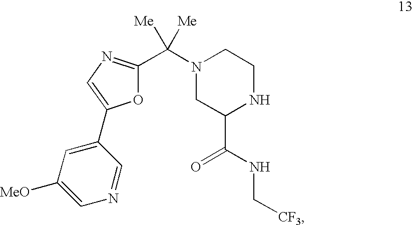

with epoxide 21:

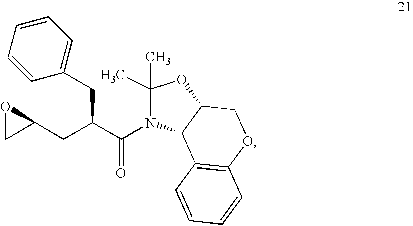

to obtain Compound 22.

Embodiments of this process include the process as just described incorporating one or more of the following features:

Compound 13 is Compound 13a:

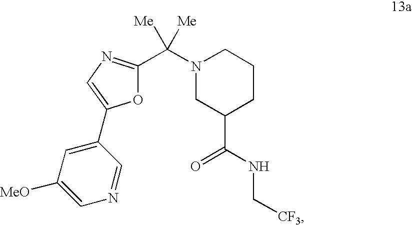

and resulting Compound 22 is Compound 22a:

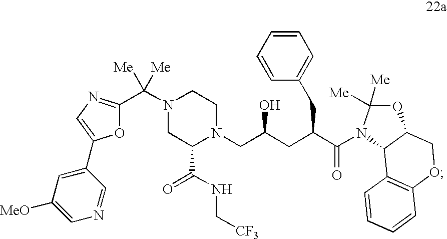

Step (ee) is conducted in a solvent selected from the group consisting of dialkyl ethers wherein each alkyl is independently a C₁-C₆ alkyl, C₁-C₆ linear and branched alkanes substituted with two —O—C₁-C₆ alkyl groups (which are the same or different), C₄-C₈ cyclic ethers and diethers, C₆-C₈ aromatic ethers, C₁-C₆ alkyl esters of C₁-C₆ alkylcarboxylic acids, C₁-C₁₀ alkyl alcohols, C₂-C₆ aliphatic nitriles, and C₇-C₁₀ aromatic nitrites;

Step (ee) is conducted in a solvent which is a C₁-C₆ alkyl alcohol;

in Step (ee) piperazine carboxamide 13 is employed in an amount in the range of from about 1 to about 3 equivalents (e.g., from about 1 to about 1.5 equivalents) per equivalent of Compound 21; or

the reaction in Step (ee) is conducted at a temperature in the range of from about 40 to about 95° C. (e.g., from about 45 to about 65° C.).

Other embodiments of the present invention include the process for preparing Compound 22 via Steps (cc), (dd) and (ee), as originally defined above, additionally incorporating any one or more of the embodiments set forth above for any one or more of Steps (cc), (dd), and (ee).

The present invention also includes a process which comprises Steps C, D and E as heretofore described, and which further comprises:

(F) treating Compound VI with acid to obtain a compound of Formula (VII):

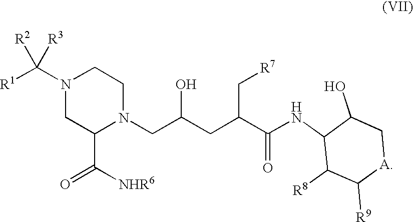

Step F is an acid deprotection step which affords Compound VII, wherein A, R¹, R², R³, R⁶, R⁷, R⁸, and R⁹ are as originally defined above in the discussion of Steps C, D and E or as defined in any of the embodiments of Steps C, D and E as set forth above. Compounds of Formula (VII) are inhibitors of HIV protease, and certain classes of the compounds encompassed by Formula (VII) (e.g., those in which R⁶=fluoroalkyl such as 2,2,2-trifluoroethyl) are inhibitors of mutant forms of HIV protease which are resistant to conventional protease inhibitors such as indinavir. These compounds are further described in WO 01/38332. Compounds representative of the classes of compounds of Formula (VII) capable of inhibiting mutant protease have exhibited IC₅₀ values below 1 nM against the wild-type enzyme and below 5 nM against the mutant enzymes Q-60, K-60, and V-18 in the assay for inhibition of microbial expressed HIV protease described in International Publication No. WO 01/38332. These compounds have also exhibited CIC₉₅ values below 50 nM against the wild-type viral construct and CIC₉₅ values below 125 nM against the viral constructs Q60, K-60, and V-18 in the cell spread assay described in WO 01/38332. These compounds are generally much more potent in both of these assays than indinavir.

In Step F, Compound VI is dissolved in a suitable solvent and brought into contact with the acid. Suitable solvents include polar organic solvents which are chemically inert under the conditions employed in Step C, such as alcohols and ethers. In one embodiment, the solvent is a dialkyl ether wherein each alkyl is independently a C₁-C₆ alkyl, C₁-C₆ linear or branched alkane substituted with two —O—C₁-C₆ alkyls (which are the same or different), a C₄-C₈ cyclic ether and diether, or a C₁-C₆ alkyl alcohol. In an aspect of this embodiment, the solvent is a C₁-C₆ alkyl alcohol (e.g., methanol).

The acid is suitably a strong acid such as the strong acids set forth above in the description of Step D. In one embodiment, the acid is trifluoroacetic acid or HCl. The acid treatment in Step F can be conducted in substantially the same manner and using the same relative proportions of acid and reactant as set forth above for the acid treatment of Step D.

The present invention further includes a process for preparing Compound 23:

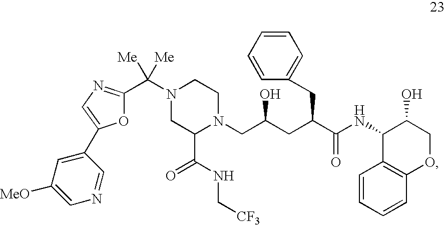

which comprises Steps (cc), (dd), and (ee) as set forth above and further comprises:

(ff) treating Compound 22 with acid to obtain Compound 23.

Embodiments of this process include the process as just described incorporating one or more of the following features:

Compound 22 is Compound 22a:

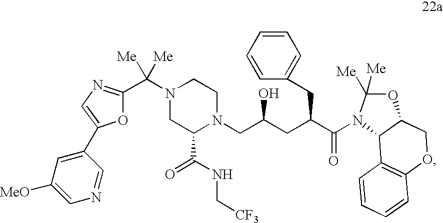

and resulting Compound 23 is Compound 23a:

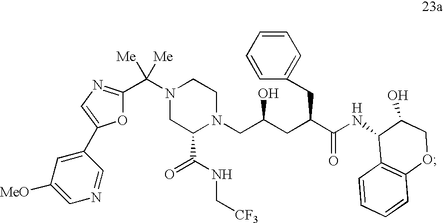

the acid in Step (ff) is an aqueous solution of HCl;

the acid in Step (ff) is a solution of HCl in a C₁-C₆ alkyl alcohol (e.g., methanol);

Step (ff) is conducted at a temperature of from about −20 to about 80° C. (e.g., in the range of from about −10 to about 10° C.); or

the acid is employed in a catalytic amount or in an amount of at least about 1 equivalent per equivalent of Compound 22.

Other embodiments of the present invention include the process for preparing Compound 26 via Steps (cc), (dd), (ee) and (ff), as originally defined above, additionally incorporating any one or more of the embodiments set forth above for any one or more of Steps (cc), (dd), (ee) and (ff).

The present invention also includes a process for preparing an iminium salt of Formula (I):

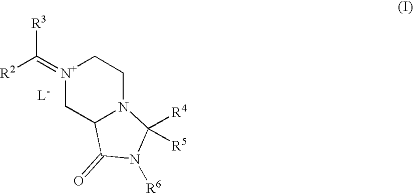

which comprises:

(A) reacting a piperazine carboxamide of Formula (VIII):

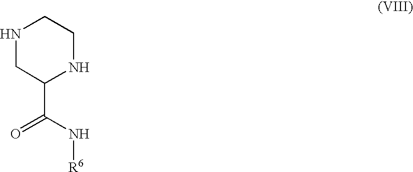

with a carbonyl-containing compound of Formula (IX):

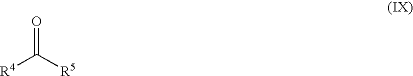

optionally in the presence of at least a catalytic amount of an acid to form an acetonide of Formula (X):

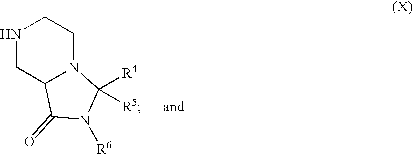

and

(B) reacting the acetonide of Formula (X) with (i) HL and a carbonyl-containing compound of Formula (XI):

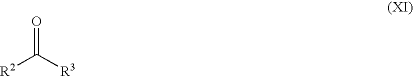

or a ketal of Formula (XI-A):

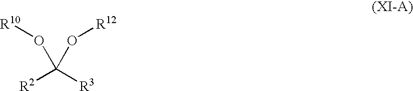

or (ii) with an alcohol of Formula (XII):

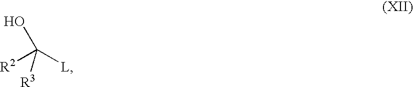

to form Compound I;

wherein L⁻, R², R³, R⁴, R⁵, and R⁶, are each as originally defined above or as defined in any one of the embodiments or aspects set forth above; and

R¹⁰ and R¹² are each independently C₁-C₄ alkyl.

It is understood that an alcohol of Formula (XII) may only be employed in Step B when the alcohol is chemically stable, which depends in large measure upon the nature of L⁻. When L⁻ is a relatively strong nucleophile such as cyanide, the cyanohydrin of Formula (XII) is typically stable and can be used as an alternative to the combination of HCN and R²C(═O)R³ or a ketal thereof. On the other hand, when L⁻ is a non-nucleophilic anion such as OTf⁻, the corresponding triflyl alcohol of Formula (XII) is typically not stable and not available for use in Step B, in which case HOTf plus R²C(═O)R³, or a ketal thereof, would be employed.

In an embodiment of this process, R¹⁰ and R¹² are both the same alkyl group (e.g., both methyl).

In another embodiment of the process, R², R³, R⁴, and R⁵ are all the same substituent which is a C₁-C₆ alkyl group. In an aspect of this embodiment, R², R³, R⁴, and R⁵ are all methyl, which means that Compounds IX and XI are both acetone and Compound XI-A is a ketal of acetone (e.g., 2,2-dimethoxypropane).

In addition to being a reactant, Compound IX (e.g., acetone) can serve as the medium for the reaction of Step A. On the other hand, a solvent can be employed to promote homogeneity, to control reaction rate by dilution and/or heat dissipation, etc. Any organic substance which will be a chemically inert liquid under the reaction conditions employed, and which will dissolve, suspend, and/or disperse the reactants can be used as the solvent, or as a co-solvent with Compound IX. The hydrocarbons, halogenated hydrocarbons, ethers, diethers, esters, and nitrites set forth above in the discussion of other reactions (see, e.g., Step C) are suitable for use as solvents and co-solvents in Step A.

The same considerations apply to Step B. That is, while Compound XI (e.g., acetone), XI-A (acetone ketal), or XII (acetone cyanohydrin) can often serve as the reaction medium for Step B in addition to its role as reactant, it can be beneficial to employ a separate substance as the solvent or as a co-solvent. Suitable solvents for Step B include those described in the preceding paragraph for Step A.

The reaction of Step A can optionally be conducted in the presence of at least a catalytic amount of an acid or a base, the choice of which is not critical. Suitable acids include, for example, HCl, HBr, sulfuric acid, tetrafluoroboric acid, phosphoric acid, nitric acid, perchloric acid. Also suitable is an organic acid selected from the group consisting of R^{u}—SO₃H, R^{v}—SO₃H, and R^{v}—CO₂H; wherein R^{u} and R^{v} are each as heretofore defined (see discussion of acid deprotection Step D). Suitable bases include those selected from the group consisting of alkali metal hydroxides, alkali metal carbonates, alkali metal oxides, C₁-C₆ alkoxides of alkali metals, alkaline earth metal hydroxides, alkaline earth metal oxides, tetra (C₁-C₄ alkyl)ammonium hydroxides, and tri-(C₁-C₄ alkyl)amines. Similarly, Step B can optionally employ at least a catalytic amount of an acid or base, wherein the acid or base may be selected from those set forth above for use in Step A.

Step A is suitably conducted at a temperature in the range of from about 0 to about 200° C. In one embodiment, the reaction is conducted at a temperature in the range of from about 20 to about 150° C. In other embodiments, the temperature is in the range of from about 30 to about 100° C., or is in the range of from about 50 to about 80° C. Similarly, Step B can be conducted at a temperature in the range of from about 0 to about 200° C., or from about 20 to about 150° C., or from about 30 to about 100° C., or from about 50 to about 80° C.

The reactants in Steps A and B can be employed in any proportion which will result in the formation of at least some of the desired product, but they are typically employed in proportions which will optimize conversion of at least one of the reactants. In Step A, piperazine carboxamide VIII is suitably employed in an amount in the range of from about 0.001 to about 10 equivalents per equivalent of Compound IX, and is typically employed in an amount in the range of from about 0.001 to about 1 equivalent per equivalent of Compound IX. Compound IX can serve the dual roles of reactant and reaction medium, in which case it is present in an excess over VIII. Accordingly, in one embodiment, piperazine carboxamide VIII is employed in an amount of from about 0.005 to about 0.5 equivalent per equivalent of Compound IX.

In Step B, each of Compounds XI, XI-A and XII is suitably employed in an amount in the range of from about 0.5 to about 100 equivalents per equivalent of acetonide X. In one embodiment, each of XI, XI-A and XII is employed in an amount in the range of from about 0.5 to about 10 equivalents (e.g., from about 0.9 to about 5 equivalents, or from about 1.0 to about 1.2 equivalents, or about 1 equivalent) per equivalent of acetonide X. HL is typically employed in an amount of equivalents equal to the equivalents of Compound XI (or XI-A).

In a suitable procedure for conducting Step A, piperazine carboxamide VIII is dissolved in an excess amount of carbonyl-containing compound IX (or both VIII and IX are dissolved in a suitable solvent) and the solution is heated to and/or maintained at a suitable reaction temperature until the reaction is complete or, alternatively, a desired amount of conversion is achieved. The resulting acetonide X can then be isolated (e.g., as a salt) by conventional procedures for use in Step B. In one embodiment, Step B comprises treating acetonide X with HL to form the acid addition salt thereof and then charging the addition salt to the carbonyl-containing compound XI or ketal XI-A, which either doubles as the reaction medium or is itself dissolved in a suitable solvent. Alternatively, acetonide X can be dissolved in XI or XI-A (or both dissolved in a suitable solvent), followed by addition of HL. The solution is heated to and/or maintained at a suitable reaction temperature until the reaction is complete or the desired degree of conversion is achieved. Similar procedures can be used for Step B when alcohol XII is employed instead of HL+XI.

The process for preparing iminium salt I via Steps A and B can be conducted in one pot by adding (i) Compound XI or XI-A and HL or (ii) alcohol XII to the pot before commencement of, during, or after completion of reaction step A, wherein

(a) when the addition of (i) Compound XI or XI-A and HL or (ii) alcohol XII to the pot is before commencement of reaction step A, reaction steps A and B are conducted concurrently in the pot;

(b) when the addition of (i) Compound XI or XI-A and HL or (ii) alcohol XII to the pot is during reaction step A, reaction steps A and B are conducted concurrently in the pot subsequent to the addition; and

(c) when the addition of (i) Compound XI or XI-A and HL or (ii) alcohol XII to the pot is after completion of reaction step A, reaction steps A and B are conducted sequentially in the pot.

The present invention also includes a process for preparing an iminium salt of Formula (I-A):

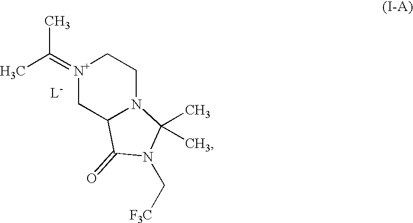

which comprises:

(aa) reacting a piperazine carboxamide 7:

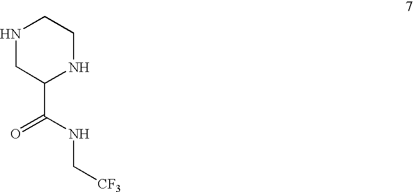

with acetone to form an acetonide 9:

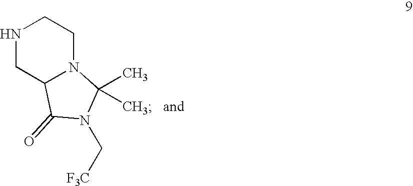

(bb) reacting acetonide 9 with (i) HL and acetone or a ketal of acetone to form I-A;

wherein L⁻ is a non-nucleophilic counterion.

Embodiments of this process include the process as just described incorporating one or more of the following features:

L⁻ is selected from the group consisting of (1) halide, (2) BF₄⁻, (3) (C₆F₅)₄B⁻, (4) MF₆⁻, wherein M is P, As, or Sb, (5) ClO₄⁻, (6) benzotriazolyl anion, (7) aryl-SO₃⁻, wherein the aryl is optionally substituted with one or more substituents each of which is independently halo, C₁-C₁₀ alkyl, or C₁-C₁₀ haloalkyl, (8) C₁-C₆ alkyl-SO₃⁻ wherein the alkyl is optionally substituted with one or more halogens, and (9) trihaloacetate anion;

L⁻ is selected from the group consisting of fluoride, chloride, BF₄⁻, (C₆F₅)₄B⁻, PF₆⁻, AsF₆⁻, SbF₆⁻, ClO₄⁻, benzotriazolyl anion, OTf⁻, CF₃CF₂SO₃⁻, C₆F₅SO₃⁻, OTs⁻, and CF₃CO₂⁻;

L⁻ is OTf⁻; such that iminium salt I-A is iminium salt 11:

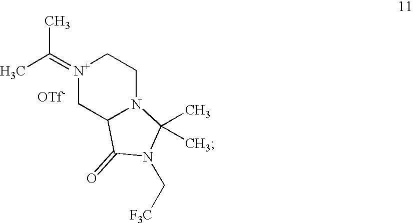

Step (aa) is conducted at a temperature in the range of from about 0 to about 200° C., or from about 20 to about 150° C., or from about 30 to about 100° C.; or from about 50 to about 80° C.;

Step (bb) is conducted at a temperature in the range of from about 0 to about 200° C., or from about 20 to about 150° C., or from about 30 to about 100° C.; or from about 50 to about 80° C.;

Step (aa) is conducted in the presence of at least a catalytic amount of an acid or a base;

Step (bb) is conducted in the presence of at least a catalytic amount of an acid or a base;

Compound 7 is employed in Step (aa) in an amount in the range of from about 0.001 to about 1 equivalent (e.g., from about 0.005 to about 0.5 equivalent) per equivalent of acetone;

acetone or the ketal of acetone is employed in an amount in the range of from about 0.5 to about 100 equivalents (e.g., from about 0.5 to about 10 equivalents, or from about 0.9 to about 5 equivalents) per equivalent of acetonide X;

the ketal of acetone is 2,2-dimethoxypropane; or

Steps (aa) and (bb) are conducted in one pot by adding acetone or acetone ketal and HL to the pot before commencement of, during, or after completion of reaction step (aa).

In an aspect of the process for preparing iminium salt I-A, Compound 7 is Compound 8:

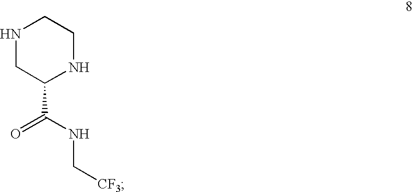

Compound 9 is Compound 9a:

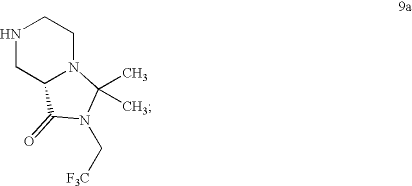

and iminium salt I-A is iminium salt I-Aa. In a feature of this aspect, when L⁻ is OTf⁻; iminium salt I-Aa is iminium salt 11a:

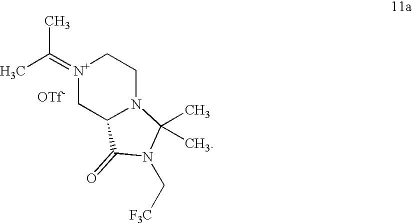

The present invention also includes a process for preparing a compound of Formula (III) via iminium salt I, which comprises Steps (A), (B) and (C) as originally defined and described above. Embodiments of this process include preparing iminium salt I via Steps (A), (B), and (C) as originally defined above, additionally incorporating any one or more of the embodiments set forth above for any one or more of Steps (A), (B), and (C). Still other embodiments includes the process comprising Steps (A), (B) and (C) and further comprising Step (D), Steps (D) and (E), or Steps (D) and (E) and (F) as heretofore defined and described.

The present invention also includes a process for preparing Compound 13 which comprises Steps (aa), (bb) and (cc) as originally defined and described above. Embodiments of this process include preparing 13 via Steps (aa), (bb), and (cc) as originally defined above, additionally incorporating any one or more of the embodiments set forth above for any one or more of Steps (aa), (bb), and (cc). Still other embodiments include the process comprising Steps (aa), (bb) and (cc) and further comprising Step (dd), Steps (dd) and (ee), or Steps (dd) and (ee) and (ff) as heretofore defined and described.

The present invention also includes a process for preparing Compound 10:

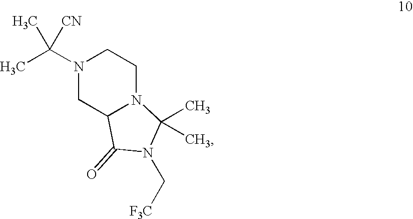

which comprises:

(aa) reacting a piperazine carboxamide 7:

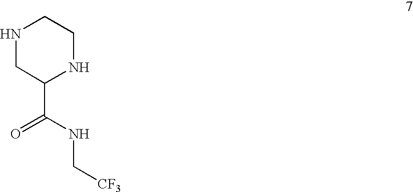

with acetone to form an acetonide 9:

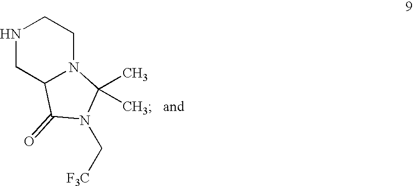

and

(bb*) reacting acetonide 9 with acetone cyanohydrin to form 10.

Embodiments of this process include the process as just described incorporating one or more of the following features:

Step (aa) is conducted at a temperature in the range of from about 0 to about 200° C., or from about 20 to about 150° C., or from about 30 to about 100° C.; or from about 50 to about 80° C.;

Step (bb*) is conducted at a temperature in the range of from about 0 to about 200° C., or from about 20 to about 150° C., or from about 30 to about 100° C.; or from about 50 to about 80° C.;

Step (aa) is conducted in the presence of at least a catalytic amount of an acid or a base;

Step (bb*) is conducted in the presence of at least a catalytic amount of an acid or a base;

Compound 7 is employed in Step (aa) in an amount in the range of from about 0.001 to about 1 equivalent (e.g., from about 0.005 to about 0.5 equivalent) per equivalent of acetone;

acetone cyanohydrin is employed in an amount in the range of from about 0.5 to about 10 equivalents (e.g., from about 0.9 to about 5 equivalents, or from about 1.0 to 1.2 equivalents) per equivalent of acetonide X; or

Steps (aa) and (bb*) are conducted in one pot by adding acetone or acetone ketal and HL to the pot before commencement of, during, or after completion of reaction step (aa).

In an aspect of the process for preparing Compound 10, Compound 7 is Compound 8, Compound 9 is Compound 9a, and Compound 10 is Compound 10a:

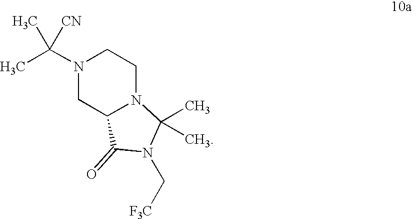

The present invention also includes a process for preparing Compound 13 which comprises Steps (aa), (bb*) and (cc) as originally defined and described above. Embodiments of this process include preparing 13 via Steps (aa), (bb*), and (cc) as originally defined above, additionally incorporating any one or more of the embodiments set forth above for any one or more of Steps (aa), (bb*), and (cc). Still other embodiments include the process comprising Steps (aa), (bb*) and (cc) and further comprising Step (dd), Steps (dd) and (ee), or Steps (dd) and (ee) and (ff) as heretofore defined and described.

The present invention also includes a compound of Formula (I-A):

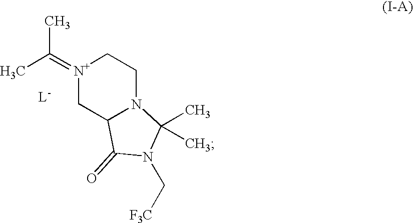

wherein L⁻ is a counterion as heretofore defined and described.

In one embodiment, Compound I-A is Compound 11:

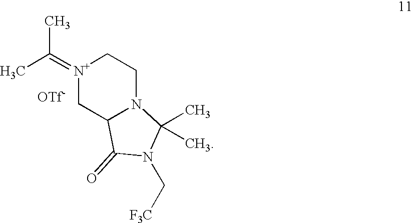

In an aspect of this embodiment, Compound 11 is Compound 11a.

In another embodiment, Compound I-A is Compound 10:

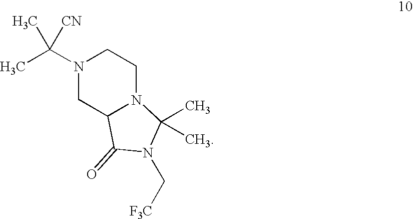

In an aspect of this embodiment, Compound 10 is Compound 10a.

The present invention also includes a compound of Formula (X):

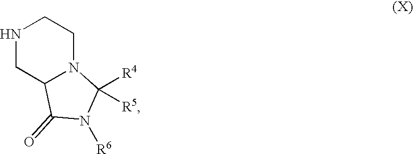

or a salt thereof; wherein R⁴, R⁵ and R⁶ are independently each as originally defined above or as defined in any one of the embodiments or aspects set forth above. Suitable salts of Compound X include include the conventional salts formed from inorganic or organic acids. In an aspect of the invention, the salts are non-toxic salts. In another aspect, Compound X is an enantiomer, such as a compound of Formula (Xa):

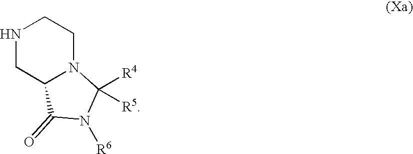

In one embodiment, Compound X is Compound 9:

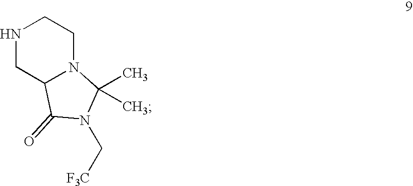

or a salt thereof. In an aspect of this embodiment, Compound 9 is Compound 9a.

Still other embodiments of the present invention include any of the processes as originally defined and described above and any embodiments or aspects thereof as heretofore defined, further comprising isolating (which may be alternatively referred to as recovering) the compound of interest from the reaction medium (e.g., iminium salt I or I-A or Compound 11, or acetonide III or III-A2 or Compound 13).

If desired, the progress of the reaction in any of the above-described chemical reactions can be followed by monitoring the disappearance of a reactant and/or the appearance of the product using TLC, HPLC, NMR, or GC.

As used herein, the term “C₁-C₆ alkyl” means linear or branched chain alkyl groups having from 1 to 6 carbon atoms and includes all of the hexyl alkyl and pentyl alkyl isomers as well as n-, iso-, sec- and t-butyl, n- and isopropyl, ethyl and methyl. “C₁-C₄ alkyl” means n-, iso-, sec- and t-butyl, n- and isopropyl, ethyl and methyl.

The term “C₂-C₆ alkenyl” refers to a linear or branched chain alkenyl group having from 2 to 6 carbon atoms, and is selected from the hexyl alkenyl and pentyl alkenyl isomers, 1-, 2- and 3-butenyl, 1- and 2-isobutenyl, 1- and 2-propenyl, and ethenyl. “C₂-C₄ alkenyl” has an analogous definition.

The term “C₂-C₆ alkynyl” refers to a linear or branched chain alkynyl group having from 2 to 6 carbon atoms, and is selected from the hexyl alkynyl and pentyl alkynyl isomers, 1-, 2- and 3-butynyl, 1- and 2-propynyl, and ethynyl. “C₂-C₄ alkynyl” has an analogous definition.

The term “C₃-C₈ cycloalkyl” refers to a cyclic ring selected from cyclopropyl, cyclobutyl, cyclopentyl, cyclohexyl, cycloheptyl and cyclooctyl. “C₃-C₆ cycloalkyl” has an analogous meaning.

The term “halogen” (which may alternatively be referred to as “halo”) refers to fluorine, chlorine, bromine and iodine (alternatively, fluoro, chloro, bromo, and iodo).

The term “C₁-C₆ haloalkyl” means a C₁ to C₆ linear or branched alkyl group as defined above with one or more halogen substituents. The term “C₁-C₄ haloalkyl” has an analogous meaning.

The term “aryl” refers herein to phenyl or naphthyl.

The term “heterocyclic” (which may alternatively be referred to as “heterocycle”) refers to (i) a 4- to 8-membered, saturated or (partially or fully) unsaturated monocyclic ring consisting of carbon atoms and one or more heteroatoms selected from N, O and S or (ii) a 7- to 10-membered bicyclic ring system, either ring of which is saturated or unsaturated, consisting of carbon atoms and one or more heteroatoms selected from N, O and S; and wherein any of the nitrogen and sulfur heteroatoms in (i) or (ii) is optionally oxidized, and any of the nitrogen heteroatoms is optionally quaternized. The heterocyclic ring may be attached at any heteroatom or carbon atom, provided that attachment results in the creation of a stable structure. Representative examples of heterocyclic groups include azetidinyl, piperidinyl, piperazinyl, azepinyl, pyrrolyl, indazolyl, pyrrolidinyl, pyrazolyl, pyrazolidinyl, imidazolyl, imidazolidinyl, imidazolinyl, pyridyl, pyrazinyl, pyrimidinyl, pyridazinyl, oxazolyl, oxazolidinyl, triazolyl, isoxazolyl, isoxazolidinyl, morpholinyl, thiazolyl, thiadiazolyl, thiazolidinyl, isothiazolyl, quinoxazolinyl, isothiazolidinyl, methylenedioxyphenyl, quinolinyl, isoquinolinyl, benzimidazolyl, thiadazolyl, benzopyranyl, benzothiazolyl, benzoazolyl, furyl, tetrahydrofuryl, benzofuranyl, benzothiofuranyl, azabenzofuranyl, benzothiazolyl, azabenzothiazolyl, azabenzoxazolyl, tetrahydropuranyl, thiophenyl (alternatively referred to herein as “thienyl”), thienothiophenyl, benzothiophenyl, and oxadiazolyl.

The term “heteroaryl” refers to a heterocyclic group as defined above, wherein the monocyclic ring (i) is an aromatic ring and in the bicyclic ring system (ii) at least one ring is an aromatic ring. In one aspect, heteroaryl refers to (i) a 5- or 6-membered aromatic ring consisting of carbon atoms and from 1 to 3 heteroatoms selected from N, S, and O or (ii) an 8- to 10-membered bicyclic ring system consisting of carbon atoms and from 1 to 3 heteroatoms selected from N, S, and O, wherein at least one of the rings in the bicyclic system is an aromatic ring. The heteroaryl ring may be attached at any heteroatom or carbon atom, provided that attachment results in the creation of a stable structure.

The term “catalytic amount” refers herein to any amount of a reagent which allows the reaction to proceed under less extreme conditions (e.g., at a lower reaction temperature) and/or in a shorter reaction time compared to the reaction conditions and/or reaction time in the absence of the reagent. A catalytic amount of a reagent is generally a substoichiometric amount of the reagent relative to the reactants, and herein is typically from about 0.001 to less than 1 molar equivalent (e.g., from about 0.001 to about 0.9 equivalent, or from about 0.01 to about 0.5 equivalent) per mole of reactant. The term “at least a catalytic amount” means that either a catalytic amount or more than a catalytic amount of the reagent can be employed. More than a catalytic amount is generally a stoichiometric amount or more than a stoichiometric amount of the reagent relative to the reactants; i.e., at least 1 molar equivalent (e.g., from about 1 to about 10 molar equivalents, or from about 1 to about 2 molar equivalents) per mole of reactant.

The term “substituted” (which appears in such expressions as “substituted with one or more substituents”) includes mono- and poly-substitution (e.g., from 1 to 5 substituents, from 1 to 4 substituents, from 1 to 3 substituents, or 1 or 2 substituents) by a named substituent to the extent such single and multiple substitution is chemically allowed and results in a chemically stable compound.

The symbol “ [symbol] ” in front of an open bond in the structural formula of a group marks the point of attachment of the group to the rest of the molecule.

Combinations of substituents and/or variables are permitted only to the extent such combinations result in chemically stable compounds under the process conditions described herein.

When any variable (e.g., R^{c}, R^{d}, or R^{e}) occurs more than one time in any constituent or in any formula, its definition on each occurrence is independent of its definition at very other occurrence.

Many of the compounds included in the present invention have at least one asymmetric center. The present invention includes all isomeric forms of each of these compounds, both individually (e.g., individual diastereomers and enantiomers) and as mixtures (e.g., racemic mixtures).

Abbreviations used in the instant specification include the following:

ACN=acetonitrile

AIDS=acquired immunodeficiency syndrome

Alloc=allyloxycarbonyl

ARC=AIDS related complex

Boc=butyloxycarbonyl

CSA=camphorsulfonic acid

DME=1,2-dimethoxyethane

DMEU=1,3-dimethyl-2-imidazolidinone (or N,N′-dimethylethyleneurea)

DMF=dimethylformamide

DMPU=1,3-dimethyl-3,4,5,6-tetrahydro-2(1H)-pyrimidinone (or N,N′-dimethylpropyleneurea)

EDC or EDAC=1-ethyl-3-(3-dimethylaminopropyl) carbodiimide

g=gram(s)

GC=gas chromatography

h=hour(s)

HIV=human immunodeficiency virus

HMPA=hexamethylphosphoramide

HOBT or HOBt=1-hydroxy benzotriazole hydrate

HIPLC=high performance liquid chromatography

IPAc=isopropyl acetate

KF=Karl Fisher titration for water

Me=methyl

min=minute(s)

MTBE=methyl tert-butyl ether

NMR=nuclear magnetic resonance

NSA=naphthalenesulfonic acid

OTf⁻=triflate anion (i.e., CF₃SO₃⁻)

OTs⁻=tosylate anion (i.e., p-Me—PhSO₃⁻)

Ph=phenyl

TBDC=di-t-butylcarbonate

TBSOTf=t-butyldimethylsilyl triflate

TFA=trifluoroacetic acid

TfOH=triflic acid

THF=tetrahydrofuran

TMEDA=N,N,N′,N′-tetramethylethylenediamine

TosMic=p-tosylmethylisocyanide

The following examples serve only to illustrate the invention and its practice. The examples are not to be construed as limitations on the scope or spirit of the invention.

### EXAMPLE 1

Preparation of 3-methoxy-5-bromopyridine

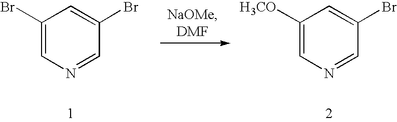

| Material | Amt | | mmol | MW |
|---|---|---|---|---|
| 3,5-Dibromopyridine | 100 | g | 422 | 236.9 |
| NaOMe* | 96.5 | mL | 422 | 54 |
| DMF** | 112 | mL | | |
| Water | 750 | mL | | |
| MTBE | 750 | mL | | |
| THF | 1000 | mL | | |

*(25 wt %, d = 0.945 g/mL)
**(KF <200 μg/mL, d 0.944 g/mL)

In a 3L 3-neck flask fitted with an overhead stirrer and a distillation head under nitrogen was charged dibromopyridine 1 (100 g) and DMF (112 mL). The solution was degassed by sparging with nitrogen for 5 min. Sodium methoxide/MeOH (25 wt % solution, 96.5 mL) was then added over 5 min and the mixture was heated to 100° C. and aged, while allowing the methanol to slowly distill, until 98 A % conversion (as measured by HPLC) was achieved. The mixture was allowed to cool to 20° C. and then quenched with 250 mL water and 500 mL MTBE. The aqueous layer was back-extracted once with 250 ml MTBE. The organic layer was washed with water (2×250 mL). The organic layer after water washes was assayed for DMF (less than about 2 mol % vs product). and then was solvent switched into dry THF via atmospheric distillation. The solvent switch was monitored by GC until <1 v/v % MTBE remained and the KF was <200 ug/mL. The final solution volume was 500 mL. The solution was assayed and found to contain 74.6 g of product 2 (94% yield).

¹H-NMR of 2: (400.13 Mhz, CDCl₃) δ 8.28 (m, 1H), 8.20 (m, 1H), 7.33 (m, 1H), 3.85 (s, 3H).

HPLC Assay:

A 50 μL sample is diluted to 50 mL (1000×) with water then acetonitrile (approximately 50:50).

| Column: | Inertsil ODS-3, 5 u, 25 cm × 4.6 mm | |
|---|---|---|
| Eluent A | water | |
| Eluent B | ACN | |
| Gradient | 90% A to 10% A over 25 min | |
| Flow | 1.0 | mL/min |
| Detection | UV at 200 | nm |
| Temp | 30° | C. |

Retention times (minutes)

| | |
|---|---|
| DMF | 3.7 |
| Methoxypyridine | 9.8 |
| Methoxypyridine carboxaldehyde | 10.4 |
| Oxazole | 11.0 |
| Bromomethoxypyridine | 16.8 |
| Dibromopyridine | 20.4 |

GC Assay:

Sample prep: 1 uL of the reaction solution was directly injected onto the column. Column: RTX 1701 30m×0.53 mm.

| Injector Temp: | 200° | C. |
|---|---|---|
| Oven Isothermal | 45° | C. for 5 min then |
| | 10° | C./min to 250° C. |
| Detector (FID) | 200° | C. |
| Retention Times (minutes) | | |
| MTBE | 1.3 | |
| THF | 2.1 | |

### EXAMPLE 2

Preparation of 3-methoxy-5-formylpyridine

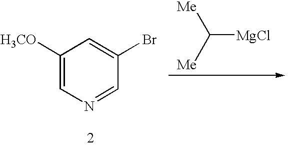

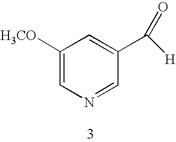

| Materials | Amt | | mmol | MW |
|---|---|---|---|---|
| Bromomethoxypyridine | 74.6 | g | 396 | 188 |
| Isopropyl MgCl (2.0M in THF) | 218 | mL | 435 | |
| DMF (d = 0.944 g/mL) | 6 | mL | 792 | 73.1 |
| THF | 180 | mL | | |
| Isopropylacetate | 290 | mL | | |
| 2N HCl | 360 | mL | 720 | |
| NaCl | 40 | g | | |

In a 2 L 3-neck flask under nitrogen fitted with overhead stirrer and an addition funnel was charged the bromomethoxypyridine solution in THF of Example 1 (75 g in about 500 ml). The system was purged with nitrogen. and then isopropylmagnesiumchloride (218 mL) was added while keeping the temperature below 20° C. The metallation was monitored by HPLC until <1 A % starting material remained (approximately 2 h), wherein the reaction was assayed using the HPLC conditions set forth in Example 1. A solution of 61 mL DMF and 180 mL THF was then added dropwise over about an hour while maintaining the temperature below 25° C. Efficient mixing and slow addition was required to maintain a stirrable slurry. The mixture was aged until >98% conversion as monitored by HPLC, about 4 h. The reaction mixture was cooled 5° C. and 3N HCl (310 mL) added while maintaining the internal temperature below 25° C. Addition of the HCl solution dissolved all the solids. The pH of the aqueous layer was 7-7.5. Solid sodium chloride (35 g) was then added and the mixture stirred at 25° C. for 20 min to achieve dissolution. The phases were separated and the aqueous layer was back-extracted with 100 ml isopropyl acetate. The overall loss to the aqueous layer was <3%. The organic layer was then solvent switched into IPAc at atmospheric pressure using 1.5 L IPAc, adjusting the volume to approx. 450 mL for use in the next step (see Example 3. Less than about 4% THF remained as assayed by GC. The solution was assayed and found to contain 48.8 g of aldehyde 3 (90% yield).

¹H-NMR of 3: (399.87 Mhz, CDCl₃) δ 10.09 (s, 1H), 8.64 (d, 1H, J=1.6 Hz), 8.53 (d, 1H, J=3 Hz), 7.59 (m, 1H), 3.90 (s, 3H).

¹³C-NMR of 3: (100.55 Mhz, CDCl₃) δ 190.7, 156.3, 145.1, 144.8, 132.1, 116.5, 55.8.

### EXAMPLE 3

Preparation of 5-(3-methoxypyrid-5-yl)-oxazole

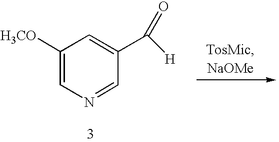

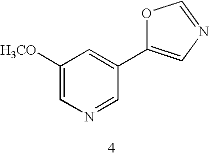

| Material | Amt | | mmol | MW |
|---|---|---|---|---|
| Aldehyde 3 | 48.8 | g | 356 | 137 |
| TosMIC | 72.9 | g | 374 | 195 |
| 25 wt % NaOMe* | 162 | mL | 712 | 54 |
| 15 wt % KCl | 500 | mL | | |
| IPAC | 900 | mL | | |
| Heptane | 470 | mL | | |
| Methanol | 450 | mL | | |

*(d = 0.945 g/mL)

In a 2L flask under a nitrogen atmosphere equipped with overhead stirrer and an addition funnel, was charged the aldehyde solution 3 (49 g in 450 mL IPAc) and then diluted with 450 mL methanol. TosMIC (72.9 g) was then added to the solution and the mixture was cooled to 0° C. To this mixture was added 2 mL of sodium methoxide solution via the addition funnel. A temperature rise of 14° C. was observed on this scale (no cooling). A heat of reaction of −44.7 Kcal/mole aldehyde was measured by calorimetry. After the cessation of the exotherm, the rest of the sodium methoxide was added (160 mL). No significant amount of rise in temperature was observed. The mixture was allowed to warm to room temperature and aged until >99% complete as judged by HPLC, about 1 h at 20° C. The reaction mixture was concentrated under vacuum (t<30° C.) to approximately a 150 mL reaction volume. Isopropyl acetate (750 mL) was then added to the thick slurry. Methanol must be removed to less than about 3% in IPAc as monitored by the GC conditions set forth in Example 1 to ensure successful partioning in the workup. To the slurry was added 300 mL of a 15% potassium chloride solution. Addition of the KCl dissolved the insoluble inorganic material. The layers were cut and the aqueous layer back extracted twice with 150 mL of IPAC. The organic layers were combined and washed with 1×200 mL of 15% potassium chloride solution. The organic layer is washed to remove sodium sulfinate. Aqueous loss of product oxazole was approximately 0.5%. The organic layer was found to contain 59 g of product (94% yield). The organic layer was then concentrated at atmospheric pressure to approximately 250 mL (200 mg/mL) and cooled to 20° C. and aged for several hours to allow for slow crystallization (seeding can optionally be employed). The IPAc solution was then switched into n-heptane by distilling at a temperature range of <50° C. under vacuum. After the solvent switch was completed as measured by GC (typically 5% IPAC in heptane) the slurry was allowed to cool to ambient temperature. The slurry was then filtered to isolate the product. The cake was washed with heptane:IPAC mixture (95:5). 56.5 g of product 4@100 wt % was obtained (90% isolated yield).

¹H-NMR of 4: (400.25 Mhz, CDCl₃) δ 8.55 (m, 1H), 8.25 (s, 1H), 7.95 (s, 1H), 7.45 (s, 1H), 7.38 (m, 1H), 3.9 (s, 3H).

¹³C-NMR of 4: (100.65 Mhz, CDCl₃) δ 155.7, 151.1, 148.7, 138.0, 137.6, 124.4, 123.0, 115.6, 55.7.

### EXAMPLE 3A

5-(3-methoxypyrid-5-yl)-2-(dimethyl-t-butylsilyl)oxazole

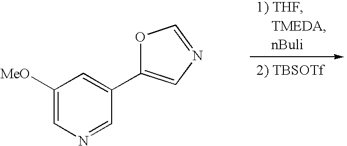

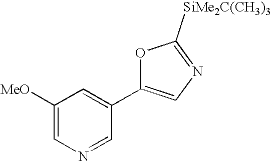

| Materials | Amt. | | mmole | MW |
|---|---|---|---|---|
| Pyridyloxazole | 2.15 | gm | 12.2 | 176.2 |
| THF | 43 | mL | | |
| nBuLi (2.5M in hexanes) | 5.1 | mL | 12.8 | 73.1 |
| TMEDA (d = 0.77 gm/mL) | 1.84 | mL | 12.2 | 116 |
| TBSOTf (d = 1.151 gm/mL) | 2.8 | mL | 12.2 | 264 |
| 5 wt % Sodium Bicarbonate | 11 | mL | | |
| IPAc | 11 | mL | | |
| Heptane | 30 | mL | | |

To the dry, degassed THF solution of pyridyloxazole (2.15 gm in 43 ml) was added TMEDA (1.84 ml) followed by 5.1 mL of nBuLi, maintaining the temperature between −15 to −5 C. After aging for 20 min, TBSOTf (2.8 mL) was added at −20C, warmed to 15C and the reaction monitored by HPLC. The reaction mixture was quenched with 11 mL 5% sodium bicarbonate, 11 mL IPAC and the layers cut. The organic layer was washed once with water and then solvent-switched into heptane. The resulting slurry was cooled to 0° C. aged and then filtered to afford 2.7 gm of the title product (9.3 mmol, 76%).

¹H-NMR (399.9 MHz, CDCl₃) δ 8.53 (m, 1H), 8.30 (m, 1H), 7.48 (s, 1H), 7.40 (m, 1H), 3.90 (s, 3H), 1.03 (s, 9H), 0.40 (s, 6H);

HPLC conditions:

| Column: | Inertsil ODS-3, 5 u, 25 cm × 4.6 mm | |
|---|---|---|
| | Metachem PN #0396 | |
| Eluent A | water (unbuffered) | |
| Eluent B | ACN | |
| Gradient | 70% A to 10% A over 20 min | |
| Flow | 1.0 | ml/min |
| Detection | UV at 200 | nm |
| Temp | 30° | C. |
| Compound | Retention Time (min) | |
| Pyridyloxazole | 6.2 | |
| TBS pyridyloxazole | 20.3 | |
| TBS pyridylisonitrile | 20.8 | |

### EXAMPLE 4

### 2(S)-((2,2,2-trifluoroethyl)aminocarbonyl)piperazine, bis-CSA Hydrate Salt

Step One: Preparation of the Pyrazine Amide

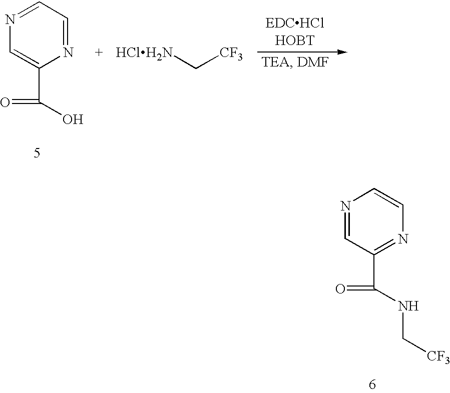

Pyrazine 2-carboxylic acid (1204 g) was suspended in DMF (4.8 L, 4 mL/g acid). 2,2,2-trifluoroethylamine.HCl (TFEA.HCl) (1200 g), 1-hydroxybenzotriazole (HOBT) (60 g) and triethylamine (TEA) (1410 mL) were then added sequentially (exotherm upon addition of TEA, flask cooled with ice bath and temperature kept below 35° C.). The reaction was cooled to 15° C. and 1-(3-dimethylaminopropyl)-3-ethylcarbodiimide.HCl (EDC.HCl) (1940 g) was added portionwise over 15-30 min. The reaction temperature was kept below 35° C. When the reaction appeared complete (approx. two hours, <5% pyrazine 2-carboxylic acid by LC assay), the reaction mixture (yellow/white slurry) was diluted with 10% K₂CO₃ in water (24 L, 20 mL/g acid) and the reaction slurry was kept below 35° C. The slurry was cooled to 10° C., aged for two hours and filtered (mother liquor assay=3-4 mg/mL). The wet cake was washed with deionized water (12 L, 10 mL/g acid) and dried under vacuum (22″ Hg) at 40° C. with a nitrogen purge. Theoretical yield of 1816 g. Actual yield 1533 g (84%).

¹H NMR: (CD₃CN, 400 MHz): δ 9.29 (d, J=1.5 Hz, 1H), 8.82 (d, J=2.5 Hz, 1H), 8.63 (dd, J=2.6, 1.4 Hz, 1H), 8.40 (bs, 1H), 4.14 (dq, J=9.4, 6.8 Hz, 2H).

HPLC Assay conditions: Waters Xterra RP8 column, elution with acetonitrile and 5 mM K phosphate adjusted to pH=8, detection at 220 nm.

Step Two: Preparation of the Piperazine Amide

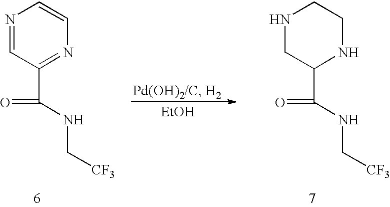

Pyrazine amide (60.2 g 0.268 mol, not corrected for water content) was suspended in absolute ethanol (550 mL) in a 1.0 L autoclave hydrogenation vessel and cooled to 15° C. Wet 20% Pd(OH)₂/C 11.0 g (20 wt %, 50 wt %wet) was added and reaction was purged with N₂ three times. H₂ (5 psig) was introduced with stirring and the temperature maintained at 15° C. for 60 min. The temperature was then increased to 60° C. and the hydrogen pressure increased to 40 psig and the reaction mixture stirred for 18 additional hours. The reaction was considered complete when conversion is >99% by LC assay. The reaction mixture was filtered through Solka-Floc and the catalyst solids were washed with ethanol 2×110 mL. Assay of the combined filtrate and washes gave 53.5 g of racemic piperazine amide (Yield=86%)

1H NMR (CD₃CN, 400 MHz): δ7.58 (bs, 1H), 3.90 (dq, J=9.5, 6.7 Hz, 2H), 3.24(dd, J=7.9, 5.5 Hz, 1H), 2.96 (dd, J=12.1, 3.6 Hz, 1H), 2.84-2.78 (m, 1H), 2.77-2.67 (m, 3H), 2.66-2.56 (m, 1H), 1.90 (s, 2H).

HPLC Assay conditions: YMC Basic column, elution with acetonitrile and 0.1% aqueous H₃PO₄, detection at 210 nm.

Step Three: Resolution of the Piperazine Amide

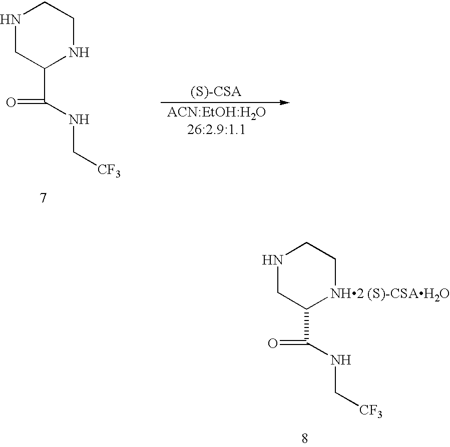

The pip amide ethanol filtrate (116.37 g containing 10.3 g of racemic pip amide by LC assay) was concentrated in vacuo to a final volume of 40.2 mL (3.9 mL per gram of pip amide) and the slurry is diluted with 82.4 mL (8 mL per gram pip amide) of acetonitrile (ACN) and stirred until homogenous. Separately (S)-camphorsulfonic acid ((S)-CSA) (19.26 g, M=232.30, 1.7 eq) was dissolved in 185 mL of ACN (18 mL per gram of pip amide). The water content of the two solutions was then determined by Karl Fisher titration. The CSA solution was added to the pip amide solution giving a small exotherm to approx. 31-32° C. Water (11.02 mL, 1.118 mL per gram of pip amide minus the total water content of the two solutions) was then added, such that the acetonitrile:ethanol:water ratio was 26:2.9:1.1 (v/v/v). Solids began to form after 15-30 min. The solution/slurry was heated to 72° C. to completely dissolve all solids. The yellow solution was recooled to 62° C. and seeded with a slurry of 10.3 mg of pip amide salt in 1 mL of acetonitrile. After a two hour age at 62° C. the slurry was allowed to cool to room temperature overnight (crystallization was complete when loss to mother liquors was <21 mg pip amide/mL by LC assay. The slurry was filtered then washed with 2×30 mL of ACN:EtOH:H₂O [(26:2.9:1.1), (v:v:v)] solution. The wet cake (˜13 g, white solid) was dried at 40° C. in a vacuum oven (24 in Hg, nitrogen sweep) to give 11.16 g of product (yield=33%). Assay method (Pip Amide) as above. Chiral assay gives an enantiomeric excess (ee) of 98.0%.

1H NMR (CD₃OD, 400 MHz): d4.84(bs, 5H), 4.64 (dd, J=12.0, 3.6 Hz, 1H), 4.13-3.94 (m, 3H), 3.77 (m, 2H), 3.66 (m, 1H), 3.54-3.43 (m, 2H), 3.28(d, J=14.7 Hz, 2H), 2.82 (d, 14.7 Hz, 2H), 2.55 (m, 2H), 2.36 (m, 2H), 2.12-1.998 (m, 4H), 1.92 (d, J=18.4 Hz, 2H), 1.72 (m, 2H), 1.45 (m, 2H), 1.09 (s, 6H), 0.87 (s, 6H). Enantiomeric excess determined by chiral HPLC of the mono BOC piperazine amide.

HPLC assay conditions: Chiral AGP column, elution with acetonitrile and 10 mM Kphospate, pH=6.5, detection at 210 nm.

Step Four: Upgrade of ee of (S)-piperazine Amide Bis (S)-CSA Salt

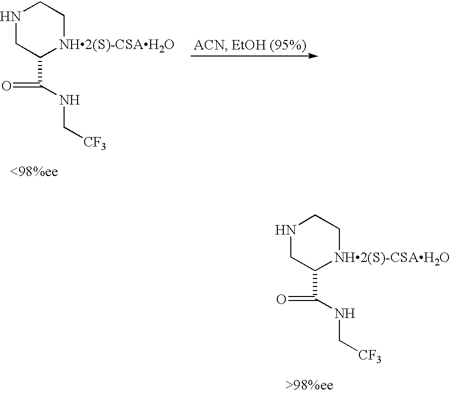

To a 12 L flask was charged (S)-pip amide salt (412.87 g) having an ee of less than 98%, 7.43 L of ACN and 825 mL of 190 proof EtOH. The slurry was heated to 75° C., aged for 1 h at 75° C. (during heating the slurry thickened considerably), then allowed to cool to 25° C. overnight. The slurry was filtered and washed with EtOH (190 proof):ACN (10:90) (2×800 mL, 2 mL/g). The white solid was dried in a vacuum oven at 24 in Hg, 40° C. with a nitrogen sweep to give 400 g of product with an ee of 99%. Assays (normal and chiral) were performed as described above in the prior steps.

### EXAMPLE 5

Piperazine trifluoroethylamide acetonide, triflate salt

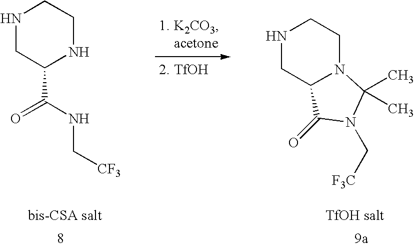

| Material | Amount | | mmol | MW |
|---|---|---|---|---|
| Piperazine trifluoroethylamide | 319 | g | 450.6 | 708 |
| bis-CSA hydrate | | | | |
| Acetone (d = 0.79 g/mL, bp = 56° C.) | 2.5 | L | 58.1 | |
| K₂CO₃ (99 + %, powdered) | 94 | g | 676 | 138.2 |
| Triflic acid (d = 1.69 g/mL, bp = 162° C.) | 40.0 | mL | 451 | 150.1 |
| IPAc (0.872 g/mL, bp = 88° C.) | 8 | L | 102.0 | |
| Heptane (d = 0.68 g/mL, bp = 98° C.) | 300 | mL | 100.2 | |

To a slurry of the bis CSA salt of the piperidine amide 8 (319 g) in acetone (1.9L) was added potassium carbonate (63 g) and the slurry heated to 50° C. for 4 hours. The reaction was monitored by NMR, and assayed by GC wherein a slurry sample of the reaction mix (approx 2 mL) was filtered through a sintered glass filter funnel followed by washing of the solid with 2 mL acetone, the resulting dried solid (KCSA waste cake) was dissolved in d-4 methanol and assayed for piperidine amide, and the concentrated filtrate was dissolved in CDCl₃ and assayed for conversion to the acetonide-protected piperidine amide. After completion of the reaction (i.e., greater than about 98% conversion to acetonide with less than about 2% piperidine in the waste cake), an additional 31 g potassium carbonate was added, aged for 30 min at 50° C. and then the reaction mixture cooled to room temperature. The KCSA was filtered off, and washed with 600 mL acetone. The combined filtrates were solvent switched into isopropyl acetate (final volume=150 mL, less than about 0.2 vol % acetone) at atmospheric pressure and then the mixture cooled to 10° C. Triflic acid (40 mL) was then added slowly maintaining the exotherm at <20° C. After a 30 min age, heptane (70 mL) was added to finish crystallization. The slurry was then filtered, washing with 200 mL 1:1 heptane:isopropyl acetate. The product was obtained as a white solid, dried initially on the filter until damp and then placed in a vacuum oven at 35° C. to afford 171 g of the title product (95% yield), wherein the product was assayed by NMR and normal phase HPLC for purity. No loss of ee was observed in title product 9a.

¹H-NMR of 9a: (400.25 Mhz, CD₃CN) 7.2 (br s, 2H), 3.93 (q, 2H, J=9.4 Hz), 2.7-3.7 (m, 7H), 1.38 (s, 3H), 1.23 (s, 3H).

¹³C-NMR of 9a: (100.65 Mhz, CD₃CN) δ 168.8, 124.1 (q, J=279.5 Hz),, 121.1 (q, J=319.5 Hz), 78.9, 55.0, 45.0, 44.6, 41.0 (q, J=35.7 Hz), 40.4, 24.1, 16.9.

GC conditions

Sample prep: 1 μL of the reaction solution was directly injected onto the column.

Column: RTX 1701 30m×0.53 mm.

| Injector Temp: | 200° | C. |
|---|---|---|
| Oven Isothermal: | 45° | C. for 5 min then |
| | 10° | C./min to 250° C. |
| Detector: | (FID) 200° | C. |
| Retention Times (min): | | |
| Acetone | 1.2 | |
| IPAc | 2.6 | |

### EXAMPLE 6

### 2(S)-((2,2,2-trifluoroethyl)aminocarbonyl)-4-(1-cyano-1-methylethyl) Piperazine Acetonide

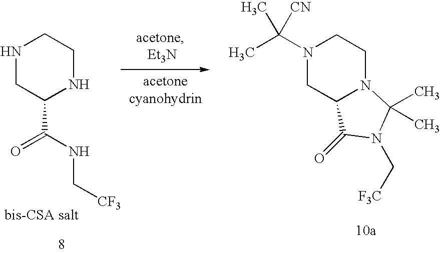

| Material | Amount | | mmol | MW |
|---|---|---|---|---|
| Piperazine trifluoroethylamide | 319 | g | 450.6 | 708 |
| bis-CSA hydrate | | | | |
| Acetone | 2000 | mL | | |
| Triethylamine (d = 0.726 g/mL) | 126 | mL | 901.1 | 101.3 |
| Acetone cyanohydrin | 42 | mL | 450.6 | 85.1 |
| (d = 0.932 g/mL) | | | | |
| Toluene | 1 | L | | |
| 10% NaHCO₃ | 500 | mL | | |

To a slurry of the his CSA salt of the piperazine amide (319 g) in acetone (2L) was added acetone cyanohydrin (42 mL) and then triethylamine (127 ml). (CAUTION: Acetone cyanohydrin is extremely toxic and liberates hydrogen cyanide with acid. Bleach should be kept ready to neutralize any spills.) The reaction mixture became homogeneous during the addition of triethylamine with only a slight endotherm. The solution was heated to reflux (approximately 60C) and aged for 12 h.

The reaction mixture was cooled to RT and the solution diluted with toluene (1 L), washed once with 10% NaHCO₃ (500 mL) and once with water (500 mL). The organic solution was concentrated to about 200 mL toluene and then solvent switched into heptane (crystallization occurs upon addition of heptane). The solution was cooled to 5C, aged 20 minutes, and then filtered, and washed with 100 mL heptane.

The product was obtained as a white solid which was dried initially on the filter until damp and then placed in a vacuum oven at 35° C. to obtain 100.0 g of 10a (70.5% yield).

¹H-NMR of 10a: (399.87 Mhz, CDCl₃) 4.0 (m, 1H), 3.8 (m, 1H), 3.35 (m, 1H), 3.25 (m, 1H), 3.1 (m, 1H), 2.95 (m, 1H), 2.65 (m 1H), 2.55 (m, 1H), 2.35 (m, 1H), 1.55 (s, 6H), 1.45 (s, 3H), 1.25 (s, 3H).

¹³C-NMR of 10a: (100.64 Mhz, CDCl₃) δ 171.3, 123.6 (q, J=279.5 Hz), 119.4, 78.0, 58.2, 56.0, 47.4, 47.3, 43.5, 41.0 (q, J=35.4 Hz), 26.5, 25.9, 24.3, 17.7.

### EXAMPLE 7

### Coupled Product

PART A -

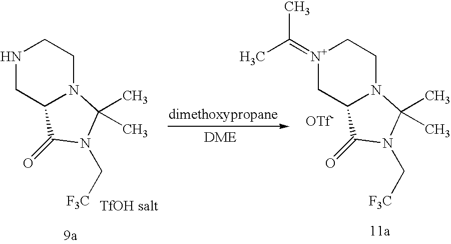

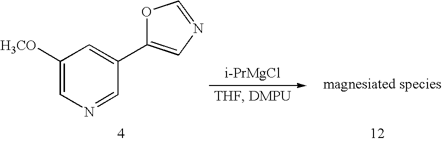

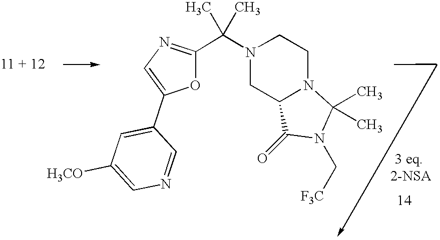

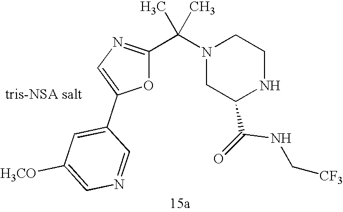

| Material | Amount | | mmol | MW |
|---|---|---|---|---|
| Trifluoroethylamide acetonide (9a), | 52 | g | 129 | 401 |
| triflate salt | | | | |
| Pyridyl oxazole (4) | 20 | g | 114 | 176 |
| Isopropyl MgCl (2.0M in THF) | 66 | mL | 132 | |
| 2,2-dimethoxypropane | 125 | mL | 1020 | 104.15 |
| (d = 0.847 g/mL) | | | | |
| DME (d = 0.867 g/mL) | 250 | mL | | 90.12 |
| IPAc (d = 0.87 g/mL) | 500 | mL | | 102.1 |
| 10% NaCl | 600 | mL | | 58.44 |
| THF (d = 0.88 g/mL) | 100 | mL | | 72.1 |
| DMPU (d = 1.060 g/mL) | 50 | mL | | 128.2 |
| 2-NSA (14) | 80.5 | g | 387 | 208 |
| ACN (d = 0.786 g/mL, bp = 82 C) | 1 | L | | 41.1 |

The triflate salt of 9a prepared as described in Example 5 (52 g) was combined with 250 mL DME and 125 mL 2,2-dimethoxypropane and heated to reflux for 30 min and then slowly distilled to remove 250 mL volatiles. The mixture containing iminium salt 11a was cooled to ambient temperature and 50 mL fresh DME added. In a separate flask was added 20 g pyridyloxazole (4) in 150 mL THF/DMPU (2:1) and the solution cooled to 0C. Isopropylmagnesium chloride (66 mL) was then added over 5 min and the solution aged for 5 h at 0° C. At the end of this age, the magnesiated species 12 was added over 15 min to the solution of iminium salt 11a, which had been pre-cooled to −10° C. To the resultant solution of the coupled product (13a) was added 500 mL of IPAc and 200 mL of 10% NaCl, and the resulting layers were separated. The organic layer was washed twice more with 10% NaCl (200 mL). The aqueous washes removed the magnesium salts and DMPU.

The organic layer containing 9a was solvent switched into 150 mL DME (<1% THF) and then 2-NSA 14 (61.1 g) was added as a slurry in water. The slurry was aged at 55-60° C. until acetonide cleavage was complete (approximately 4 h). The solution was then dried by distillation of DME until approximately 2% water remained and the mother liquor concentration of biarylpiperazine 15a was <4 mg/mL. The slurry was cooled to 20° C. and filtered, and the solid washed with DME. The tris 2-NSA salt of 15a was obtained as an off-white solid (92 g, 90% yield).

In the above-described preparation, acetonide 9a was employed in an enantiomerically pure form (i.e., >98% ee), and product 15a was obtained without loss of ee. Iminium salt 11a is a stable, isolatable substance. The magnesiated oxazole 12 is not a stable, isolatable substance.

Iminium Salt 11a—

¹H-NMR (500.13 MHz, CD₂ Cl₂) 4.61 (m, 1H), 4.45 (m, 1H), 3.94 (m, 1H), 3.85 (m, 1H), 3.75 (m, 1H), 3.67 (m, 1H), 3.17 (dt, J=11.3, 3.2 Hz), 3.01 (td, J=11.3, 2.8 Hz), 2.65 (s, 3H), 2.64 (s, 3H), 1.45 (s, 3H), 1.28 (s, 3H).

¹³C-NMR (125.76 MHz, CD₂ Cl₂) δ 191.9, 169.4, 124.2 (q, J=279.9 Hz), 79.5, 57.3, 53.9, 53.8, 42.7, 41.9 (q , J=36 Hz), 26.5, 26.4, 24.7, 19.3.

Magnesiated Oxazole 12—

¹H-NMR (600.13 MHz, d8-THF) 8.35 (m, 1H), 8.11 (m, 1H), 7.98 (m, 1H), 5.45 (m, 1H), 3.6 (s, 3H).

¹³C-NMR (150.90 MHz, d8-THF) δ 164.1, 162.9, 156.9, 139.4, 138.6, 138.0, 117.9, 83.8, 56.6.

Tris 2-NSA Salt of 15a—

¹H-NMR (600.13 MHz, CD₃OD) 8.83 (d, J=1.5 Hz, 1H), 8.48 (d, J=2.6 Hz, 1H), 8.37 (dd, J=2.6, 1.5 Hz), 7.97 (s, 1H), 7.79 (m, 6H), 7.40 (m, 9H), 4.44 (dd, J=10.6, 3.4 Hz, 1H), 4.05 (s, 3H), 4.00 (m, 1H), 3.91 (m, 2H), 3.68 (dt, J=13.6, 3.4 Hz, 1H), 3.61 (brd, J=13 Hz, 2H), 3.47 (m, 1H), 3.25 (dd, 12.8, 11.0 Hz, 1H), 3.19 (m), 1.88 (s, 6H).

¹³C-NMR (150.90 MHz, CD₃CN) δ 166.8, 165.0, 160.0, 148.1, 146.0, 131.5, 131.3, 129.8, 129.7, 129.4, 128.8, 126.9, 126.7, 125.5 (q, J=277.7 Hz), 64.1, 58.2, 56.95, 48.0, 45.2, 42.7, 41.6 (q, J=36 Hz), 23.4.

Part B—

Nitrile 10a prepared as described in Example 6 was dissolved in dichloromethane at 20° C. and treated with one equivalent of tert-butyldimethylsilyltriflate. After 20 min of aging the iminium salt 11a was fully formed as analyzed by NMR. The iminium salt 11a was then reacted with magnesiated species 12 in the manner described in Part A of this Example to obtain coupled product 13a.

### EXAMPLE 8

### Coupled Product

Triflate salt of 9a prepared as described in Example 5 was combined with 250 mL DME and 125 mL 2,2-dimethoxypropane and heated to reflux for 30 min and then distilled to remove 250 mL volatiles. A mixture containing iminium salt 11a prepared as described in Part A of Example 7 was cooled to ambient temperature and 50 mL fresh DME added. In a separate flask was added 20 g pyridyloxazole (4) in 150 mL THF/DMPU (2: 1) and the solution cooled to 0° C. Isopropylmagnesium chloride (66 mL) was then added over 5 min and the solution aged for 5 h at 0° C. At the end of this age, the magnesiated species 12 was added to the solution of iminium salt 11a, pre-cooled to −10° C. The resultant solution of the coupled product (13a) was then treated with concentrated HCl to obtain the desired product 15a (34 g; 70% yield).

### EXAMPLE 9

### Tris-naphthalenesulfonic Acid Salt of 15a

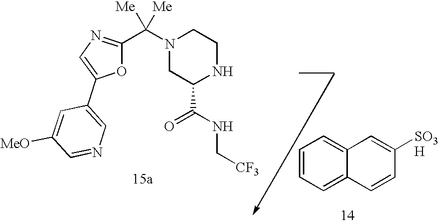 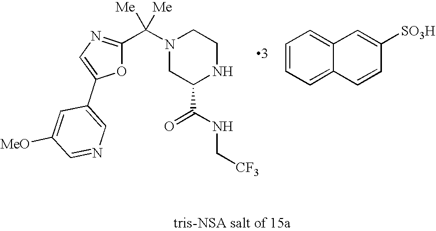 Part A - Purification of 2-Naphthalenesulfonic acid

| Run 1 - | | | | |
|---|---|---|---|---|
| Material | MW (g/mol) | Amount | | mmoles |
| 2-NSA 14 (76 wt % and 88A%) | 208.24 | 200 | g | 730 |
| 2-NSA 14 seed | | 0.30 | g | |
| Acetonitrile | | 800 | mL | |
| Water | | 10 | mL | |
| Toluene | | 950 | mL | |

The impurities present in the crude 2-NSA 14 included 1-NSA, naphthalene, two isomers of naphthalenesulfone, and sulfuric acid. The crude 2-NSA 14 (200 g) was mixed with 400 mL CH₃CN, 10 mL water and 800 mL toluene and heated to 78-80° C. to dissolve the solids. The two layers were allowed to settle and the lower black layer (about 100 mL) was cut at 80° C. The top layer was cooled and seeded at 40° C. (100 mg seed). A slurry formed at ˜33° C. The slurry was cooled to 6° C., rinsed with 150 mL toluene and air dried in the funnel to afford 169 g of acid 14. In the black cut, most of 1-naphthalenesulfonic acid and sulfuric acid were rejected. In the mother liquor most of naphthalene and isomers of naphthalenesulfone are rejected. The purity of the filtered crystals was ˜98.6 A %.

The crystals were mixed with 340 mL CH₃CN and heated to 50° C. to form a clear, gray solution, which was cooled and seeded at 40° C. (200 mg seed). A slurry formed at ˜26° C. This was cooled to 5° C., filtered and rinsed with 100 mL CH₃CN to afford after drying in a vacuum oven at 60° C., 76.8 g solid (99.8A %, 94.3 wt. % with 8% water, 48% recovery based on 76 wt % pure crude acid).

¹H NMR of 14 (DMSO-d6, δ) 8.17 (s, 1H), 7.98˜7.96 (m, 1H), 7.91˜7.90 (m, 1H), 7.88˜7.86 (m, 1H), 7.73˜7.71 (m, 1H), 7.53˜7.51 (m, 2H), 6.98 (broad, 3H).

HPLC Assay:

| Column: | Zorbax RX-C8 (4.6 mm × 250 mm) | |
|---|---|---|
| Solvents: | 50% CH₃CN, 50% 0.1% H₃PO₄ | |
| Flow: | 1.0 | mL/min |
| Sample volume: | 10 | μL |
| Wavelength: | 210 | nm |
| Retention times (min): | | |
| NSA isomer | 2.7 | |
| 2-NSA 14 | 3.1 | |
| Toluene | 10.4 | |
| Naphthalene | 13.5 | |
| Sulfone impurity #1 | 25.5 | |
| Sulfone impurity #2 | 29.2 | |
| Run 2- | | |

Crude 2-NSA 14 (40 g; from Rutgers Organic Corp.; 88 A % and 76.6 wt. % pure) was mixed with 80 mL of acetonitrile and 320 mL of toluene. The mixture was heated to about 80-82° C. to dissolve all of the solid. The mixture was maintained at temperature and allowed to settle and form two layers. The bottom black layer (13.3 g containing about 13.6% of the acid) was cut. Water (2 mL) was added to the top layer, the mixture agitated and then allowed to cool to room temperature resulting in the formation of a slurry which was aged at room temperature overnight. The slurry was filtered and rinsed with toluene (50 mL) to afford a gray solid, which was vacuum dried at 60° C. to give 27.55 g of solid (98.1 A % and 90.0 wt. % pure). Recovery was 80.8%. 6.5% of the acid was lost in the mother liquor.

Run 3—

Crude 2-NSA 14 (40 g; from Rutgers Organic Corp.; 88 A % and 76.6 wt. % pure) was mixed with 80 mL of acetonitrile and 240 mL of toluene. The mixture was heated to about 80-82° C. to dissolve all of the solid. The mixture was maintained at temperature and allowed to settle and form two layers. The bottom black layer (13.73 g containing about 14.6% of the acid) was cut. Water (30 mL) was added to the top layer, the mixture agitated and then allowed to cool to room temperature and to settle which resulted in the formation of 2 layers. The top layer most of the organic impurities was cut (0.4 wt. % of the acid was lost). The bottom layer was concentrated to about 60 mL by vacuum distillation at less than 50° C. Acetonitrile (570 mL) was then slowly added to remove the water by continuous distillation. The final volume was about 60 mL. Tolume (20 mL) was added and the mixture heated to 60° C. providing a clear solution, which was then cooled to 45° C. and seeded with 2-NSA seed crystals which resulted in the formation of a slurry which was cooled to about 0-5° C. and aged for 30 minutes. The slurry was then filtered and rinsed with toluene (30 mL) to afford an off-white solid. After vacuum drying at 60° C., a solid acid was obtained (20.9 g, HPLC: 99.5A % and 96.7 wt. % pure). Recovery was 66%. 21.5% of the acid was lost in the mother liquor.

Part B - Preparation of the Tris-NSA salt of 15a

| Material | MW (g/mol) | Amount | | moles |
|---|---|---|---|---|
| Biarylpiperazine 15a* | 427.42 | 2321 | g | 5.43 |
| Seed of tris-salt of 15a | 1052.14 | 12 | g | |
| 2-Naphthalenesulfonic acid 14** | 208.24 | 3683 | g | 16.29 |
| Acetonitrile | | 130 | L | |
| Water | | 8.9 | L | |

*15.3 wt % solution in CH₃CN, 94.8ee%
**92.1 wt % and 99.8 A%

2-Naphthalenesulfonic acid 14 (92.1 wt % pure) was dissolved in 21 L of CH₃CN and 8.82 L water at 65° C. A clear solution of biarylpiperazine 15a in CH₃CN (15.17 kg, 2.321 kg free base 6) was added over 1 min along with 1 L CH₃CN rinse. The mixture was still a clear solution (57° C.). After seeding (12 g), a slurry formed gradually. The slurry was aged 1 h at 50-60° C. The slurry was vacuum distilled at 30-45° C. and 94 L CH₃CN was added slowly to reduce the water content in order to lower the solubility of the tris-NSA salt of 15a. Samples were taken during distillation to monitor the change:

| | Volume of | | Free base in | KF value of |
|---|---|---|---|---|
| Sample | CH₃CN added | ee % of salt | supernatant | supernatant |
| #1 | 72 L | 99.9% | 3.25 g/L | 6.8% |
| #2 | 84 L | 99.8% | 2.53 g/L | 4.6% |
| #3 | 94 L | 98.2% | 1.48 g/L | 3.5% |

The volume was adjusted to ˜49 L. The slurry was cooled to 25° C. and was aged overnight. The solids were filtered, rinsed with 12 L CH₃CN and dried in a vacuum oven at 60° C. to afford 5.29 kg crystalline solid salt of 15a (99.5 A %, 41.2 wt %, 98.1 ee %, 94% recovery or 97% after ee % correction.) Loss in the mother liquor was 2.6%.

¹H NMR of the tris-NSA salt of 15a (DMSO-d6, with two drops of D2O, δ) 9.27 (t, 1H, J=6.3 Hz), 8.65 (d, 1H, J=1.6 Hz), 8.42 (d, 1H, J=1.7 Hz), 8.13 (d, 3H, J=0.8 Hz), 7.96˜7.94 (m, 3H), 7.91˜7.89 (m, 5H), 7.88˜7.85 (m, 3H), 7.72˜7.69 (m, 3H), 7.53˜7.51 (m, 6H), 4.03˜3.97 (m, 3H), 3.93 (s, 3H), 3.33˜3.24 (m, 2H), 3.02˜2.96 (m, 2H), 2.50˜2.45 (m, 2H), 1.56 (s, 6H).

HPLC Assay:

| Column: | Zorbax RX-C8 (4.6 mm × 250 mm) | |
|---|---|---|
| Solvents: | 60% CH₃CN, 40% 0.1% H₃PO₄ | |
| Flow: | 1.0 | mL/min |
| Sample volume: | 10 | μL |
| Wavelength: | 210 | nm |
| Retention times (min): | | |
| Biarylpiperazine 15a | 2.1 | |
| 2-NSA 14 | 3.1 | |

### EXAMPLE 10

Acetonide 18

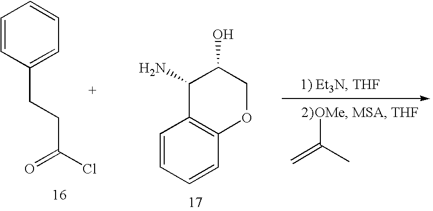

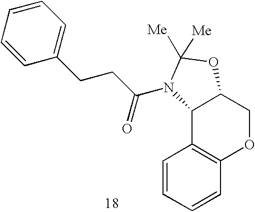

| Material | MW (g/mol) | Amount | | mmoles |
|---|---|---|---|---|
| Aminochromanol 17 | 165.19 | 100.00 | | 605 |
| Acetonide 18 | 337.41 | | | |
| Triethylamine (d = 0.726) | 101.19 | 98 | mL | 703 |
| Hydrocinnamoyl chloride 16* | 168.82 | 93 | mL | 622 |
| 2-Methoxypropene (d = 0.753) | 72.11 | 232 | mL | 2.42 |
| Methanesulfonic acid (d = 1.481) | 96.10 | 4.0 | mL | 62 |
| THF | | 2000 | mL | |
| IPAc | | 3000 | mL | |
| 5% Sodium bicarbonate | | 1800 | mL | |
| Cyclohexane | | 3850 | mL | |
| Water | | 900 | mL | |

*98%, d = 1.13

To a mixture of aminochromanol 17 (100.0 g, 95% ee, 605 mmol), TEA (89 mL, 635 mmol), and 1800 mL dry THF at room temperature was added a solution of hydrocinnamoyl chloride 16 (93 μL, 622 mmol, 1.03 eq) in THF (200 ML) over 40 min, allowing the temperature to drift up to 45° C. At the end of the addition, a slurry was generated which was aged at 45° C. for 30 min then cooled to 30° C. 2-Methoxypropene (232 mL, 4.0 eq) was added, followed by 4.0 mL methanesulfonic acid (0.10 eq). The mixture was aged at 35˜38° C. for 1 h. The flask was fitted with a condenser, and the slurry was warmed to 40° C., aged for 2 h, heated to 60° C. and aged at 60° C. under N₂ for 2˜4 h until HPLC showed <0.1 A % amide remaining. The reaction was quenched with 9 mL triethylamine. The mixture was concentrated to about 2 L by vacuum distillation at <60° C. IPAc (3 L) was added slowly to replace THF. The final volume was 2.4 L. The mixture was cooled to room temperature and 900 mL of 5% NaHCO₃ were added to dissolve all solids. After settling, the aqueous layer was cut and the organic layer was washed with 900 mL 5% NaHCO₃ and then 900 mL water. The organic layer was concentrated to 2.5 L by vacuum distillation at <85° C., and cyclohexane (3.6 L) was added slowly during distillation to solvent switch. Some solids formed during distillation. When the mixture was heated to 70˜75° C., most of the solids dissolved. At the end of distillation all solid was dissolved by heating to 75˜80° C. The clear solution was cooled slowly to room temperature over 2.5 h during which slurry formed. This was aged 30 min at room temperature and 30 min at 0˜5° C. The slurry was filtered and the solids were rinsed with 250 mL cyclohexane. After vacuum oven drying at 50° C., 184.16 g (98.7 A %, 98.1 wt % pure) of acetonide 18 was obtained. There was 6.4% loss in the mother liquor. The yield after purity correction was 88%.

¹H NMR (CDCl₃, 300 MHz) 7.25 (m, 7H), 6.82 (m, 2H), 4.70 (d, 1H), 4.33 (m, 1H), 4.08 (d, 1H), 3.92 (s, 1H), 3.11 (m, 2H), 2.92 (m, 1H), 2.68 (m, 1H), 1.61 (s, 3H), 1.23 (s, 3H).

HPLC Assay:

| Column: | Zorbax RX-C8 (4.6 mm × 250 mm) | |
|---|---|---|
| Solvents: | 60% CH₃CN, 40% 0.1% H₃PO₄ | |
| Flow: | 1.0 | mL/min |
| Sample volume: | 10 | μL |
| Wavelength: | 210 | nm |
| Retention times (min): | | |
| Aminochromanol 17 | 2.1 | |
| Hydroxyamide | 3.9 | |
| IPAc | 4.1 | |
| Acetonide 18 | 7.7 | |
| Ester impurity | 11.1 | |

### EXAMPLE 11

Olefin 19

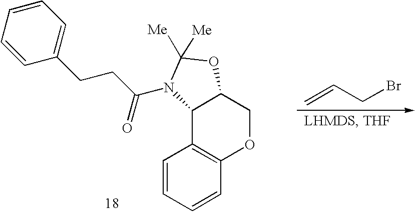

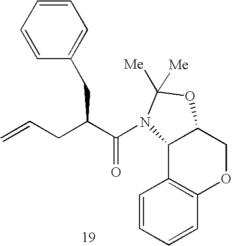

| Material | MW (g/mol) | | Amount | | mmoles |
|---|---|---|---|---|---|
| Acetonide 18 (99.6 wt. %) | 337.41 | | 50.00 | g | 148 |
| Olefin 19 | 377.48 | | | | |
| Allylbromide | 120.98 | | 18.60 | g | 154 |
| 1.38M LHMDS in THF (d = 0.89) | | | 109 | g | 169 |
| Citric acid | 192.13 | g/mol | 8.63 | g | 44.9 |
| Tetrahydrofuran | | | 343 | mL | |
| Isopropyl acetate | | | 1100 | mL | |
| 0.3M Sulfuric acid | | | 180 | mL | |
| 5% Sodium bicarbonate | | | 180 | mL | |
| Water | | | 180 | mL | |

Acetonide 18 (50.00 g, 148 mol) was dissolved in 283 mL THF (KF=116 μg/mL). The solution was degassed and was placed under N₂. The solution was cooled to −46 to −44° C. and 18.60 g (1.04 eq) allylbromide was added. LHMDS/THF (107 g) was charged over 45 min at 46 to −44° C. After a 60 min age at this temperature, a sample was taken (quenched into 2 vol cold IPA) for HPLC assay, which showed 0.68 A % acetonide 18 remaining (99.3% conversion). More LHMDS/THF (2.14 g) was added, and the mixture was aged for 30 min more. HPLC showed 0.22 A % acetonide 18 (99.8% conversion). The reaction was quenched by adding cold citric acid solution in THF (8.63 g/60 mL THF). A slurry formed. The slurry was warmed from −32° C. to 16° C. over 1 h. The batch was vacuum distilled to ˜400 mL at <40° C. and was flushed with 1100 mL IPAc to solvent switch to IPAc. The final volume was 450 mL. To the slurry was charged 180 mL 0.3 M H₂SO₄ (d=1.016 g/mL) at 20-25° C. All solids dissolved. After settling, the aqueous layer was cut and the organic layer was washed with 180 mL water and then 180 mL 5% NaHCO₃. The organic layer was diluted to 500 mL with IPAc. By HPLC the solution yield of olefin 19 was 98%. The concentration of olefin 19 was about 0.3 M. The solution was used in Example 12 without further purification.

¹H NMR (CDCl₃, 300 MHz) indicated a 5:1 mixture of rotamers: 7.30 (m, 5H), 7.05 (m, 1H), 6.80 (m, 1H), 6.4 (m, 1H), 5.85 (m, 1H), 5.15 (m, 1H), 4.98 (m, 1H), 4.40 (m, 1H), 4.25 (m, 2H), 3.38 (dd, 1H), 3.19 (m, 1H), 2.80 (m, 1H), 2.42 (m, 1H), 1.70 (s, 3H), 1.23 (s, 3H).

HPLC Assay:

| Column: | Zorbax RX-C8 (4.6 mm × 250 mm) | |
|---|---|---|
| Solvents: | 60% CH₃CN, 40% 0.1% H₃PO₄ | |
| Flow: | 1.0 | mL/min |
| Sample volume: | 10 | μL |
| Wavelength: | 210 | nm |

Retention times (min):

| | |
|---|---|
| IPAc | 4.1 |
| Allylbromide | 5.2 |
| Acetone eliminated impurity | 6.0 |
| Acetone adduct | 7.2 |
| Acetonide 18 | 7.7 |
| Olefin 19 | 12.6 |
| Epi-olefin | 12.9 |

### EXAMPLE 12

Iodohydrin 20

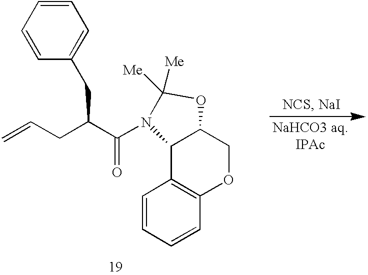

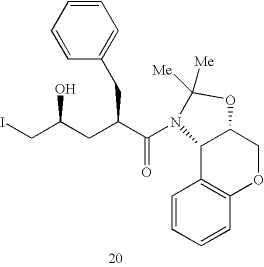

| Material | MW (g/mol) | Amount | | mmoles |
|---|---|---|---|---|
| Olefin 19 | 377.48 | | | ˜148 |
| Iodohydrin 20 | 521.39 | | | |
| NCS | 133.53 | 33.60 | g | 252 |
| 57% NaI | 149.89 | 64.22 | g | 244 |
| 20% Na thiosulfate pentahydrate | 248.18 | 165 | mL | |
| IPAc | | ˜50 | mL | |
| 5% Sodium bicarbonate | | 220 | mL | |
| Water | | 220 | mL | |

To the solution of olefin 19 (500 mL, ˜148 mmol) in IPAc was charged 220 mL water and 220 mL 5% NaHCO₃. The mixture was cooled to 3-4° C. NCS (33.60 g, 252 mmol, 1.7 eq) was added, then 57% NaI solution (64.22 g, 244 mmol, 1.65 eq) was added over 40 min at 4-7° C. The resulting brown solution was allowed to warm to 20° C. over 2 h and then was warmed to 30° C. over 15 min. The mixture was aged at 30° C. for 4 h. The conversion to iodohydrin was 98.6% after warming to 20° C. and 99.9% after 4 h age at 30° C. The batch was cooled to room temperature and then quenched with fast addition of 165 mL 20% Na₂S₂O₃.5H₂O (d=1.17 g/mL). After agitating for 2 min, the color of reaction mixture changed to orange from brown. The mixture was settled and the aqueous layer (650 mL) was cut. The organic layer (520 mL) was assayed and solution yield of iodohydrin was 83%. The solution was used in Example 13 without further purification.

¹H NMR (CDCl₃, 300 MHz) indicated a 5:2 mixture of rotamers: 7.30 (m, 5H), 7.05 (m, 1H), 6.82 (m, 1H), 6.60 (m, 1H), 5.92 (d, 0.3H), 5.58 (d, 0.7H), 4.45 (m, 2H), 4.20 (m, 2H), 3.63 (m, 1H), 3.44 (m, 2H), 3.20 (m, 2H), 2.82 (m, 2H), 2.40 (d, 1H), 2.00 (m, 1H), 1.72 (s, 3H), 1.49 (d, 2H), 1.29 (s, 3H).

HPLC Assay:

| Column: | Zorbax RX-C8 (4.6 mm × 250 mm) | |
|---|---|---|
| Solvents: | 60% CH₃CN, 40% 0.1% H₃PO₄ | |
| Flow: | 1.0 | mL/min |
| Sample volume: | 10 | μL |
| Wavelength: | 210 | nm |
| Retention times (min): | | |
| IPAc | 4.1 | |
| Iodohydrin 20 | 9.3 | |
| Olefin 19 | 12.6 | |

### EXAMPLE 13

Epoxide 21

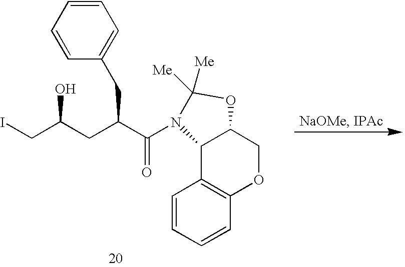

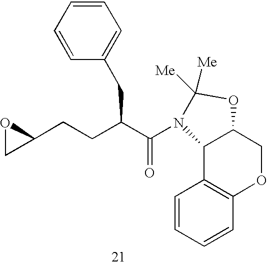

| Material | MW (g/mol) | Amount | | mmoles |
|---|---|---|---|---|
| Iodohydrin 20 | 521.39 | | | <148 |
| Epoxide 21 | 393.48 | | | |
| 25% NaOMe in MeOH | 54.02 | 44.8 | g | 207 |
| IPAc | | 500 | mL | |
| IPA | | 450 | mL | |
| 10% Sodium sulfate decahydrate | | 340 | mL | |
| Water | | 170 | mL | |

The solution of iodohydrin 20 in IPAc (520 mL, <148 mmol) was vacuum distilled at <35° C. IPAc (500 mL) was added slowly while the volume of solution was maintained at 500 mL. KF of the solution was <1400 μg/mL after the distillation. After azeotropic drying, the organic solution was cooled to 14-16° C. Then 44.8 g 25% NaOMe in methanol was added (small endotherm). The mixture was aged at 15° C. for 45 minutes. Sampling after 30 minutes age at 14-16° C. showed >99.7% conversion to epoxide. The reaction was quenched at 15-20° C. by adding 170 mL water. The mixture was agitated 2 minutes and settled 10 minutes. The aqueous layer was cut. The clear, dark brown organic layer was washed by 2×170 mL 10% Na₂SO₄— 10H₂O (d=1.04 g/mL). The pH of the first wash aqueous solution was 7 and was 6.5 for the second wash. The loss of epoxide in these two washes was <0.1%. The organic layer showed a lower 99.3% conversion to epoxide, due to some reverse reaction to iodohydrin. The organic layer was vacuum distilled to 220 mL and then flushed with 400 mL IPA at <45° C. A slurry was generated during this solvent switch. The slurry was heated rapidly to 80° C. to dissolve all solid. The dark solution was cooled slowly to 60˜65° C. and was aged at this temperature to obtain a thin slurry. The slurry was cooled to room temperature over 1 h and was cooled to 0˜5° C. for 3 h. The slurry was filtered and the cake was displacement-rinsed with 50 mL cold IPA. By HPLC there was 2.4% epoxide lost in mother liquor and rinse (160 mL). The cake was vacuum oven dried overnight at 40° C. with a nitrogen sweep to afford 48.02 g of epoxide 21 (99.4A % and 98.5 wt % pure). The yield was 81% from acetonide 20.

¹H NMR (CDCl₃, 300 MHz) indicated a 5:2 mixture of rotamers: 7.30 (m, 5H), 7.10 (m, 1H), 6.82 (m, 1H), 6.50 (m, 1H), 5.89 (d, 0.3H), 5.40 (d, 0.7H), 4.40 (m, 2H), 4.15 (m, 2H), 3.40 (m, 2H), 3.00 (m, 1H), 2.85 (m, 2H), 2.50 (dd, 0.7H), 2.40 (dd, 0.3H), 2.20 (m, 1H), 1.72 (s, 3H), 1.49 (d, 1H), 1.29 (s, 3H).

HPLC Assay:

| Column: | Zorbax RX-C8 (4.6 mm × 250 mm) | |
|---|---|---|
| Solvents: | 60% CH₃CN, 40% 0.1% H₃PO₄ | |
| Flow: | 1.0 | mL/min |
| Sample volume: | 10 | μL |
| Wavelength: | 210 | nm |
| Retention times (min): | | |
| IPAc | 4.1 | |
| Epoxide 21 | 8.0 | |
| Iodohydrin 20 | 9.3 | |

### EXAMPLE 14

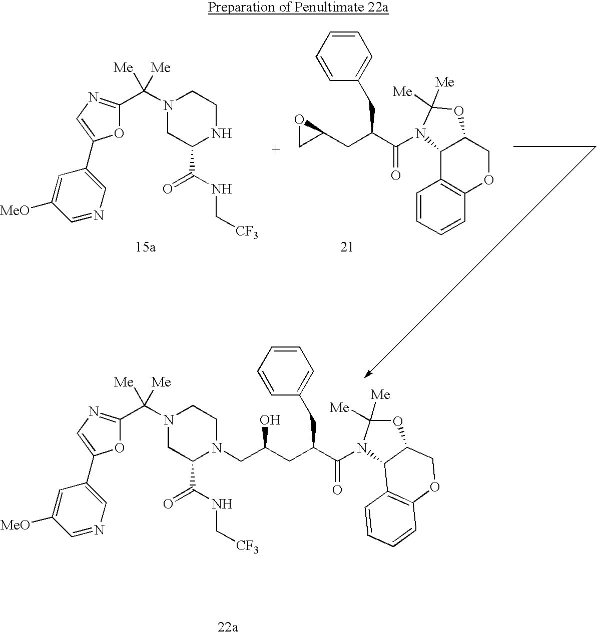

Biarylpiperazine tris-NSA salt (300.00 g, GMP) was slurried in MeOH (940 mL) and KOH in MeOH (860 mL, 1.0N). The slurry was allowed to stir for 4 h. MeOH was distilled off at 35 Torr with an internal temperature of 5° C. After ˜800 mL was distilled off, the slurry became too thick to stir and toluene (1800 mL) was added. A total of 3600 mL of toluene was used to flush the slurry (mother liquors were checked for the presence of naphthalenesulfonic acid). The slurry was then filtered, rinsing 2×360 mL toluene. The filtrates were assayed by HPLC and found to contain 123.1 g biarylpiperazine. The filtrate was then concentrated and diluted with 480 mL t-amyl alcohol. It was concentrated again and then flushed with 450 mL t-amyl alcohol. It was assayed and found to contain 115.1 g biarylpiperazine. Epoxide 21 (107.00 g, 1.01 eq.) was added, and the mixture was stirred at 55° C. (internal temperature) for 90 h. The mixture was diluted with IPAc (1720 mL) and assayed for the coupled acetonide product 22a by HPLC (found 185.00 g (84% yield). Silica gel (370.0 g) and Darco G-60 activated carbon (46.25 g) were added and the mixture was heated at 50° C. for 1 hour. It was filtered through Solka Floc and rinsed with 925 mL 5% MeOH/IPAc (4×). The initial filtrate and first rinse were assayed and were found to contain a total of 146.03 g. Rinses 3 and 4 contained 24.69 g and 7.52 g, respectively. The filtrate, first and second rinses were combined. A portion of this containing 100 g was chromatographed (16 cm column, 2.00 kg silica) using 0 to 6% MeOH/IPAc. Clean fractions were combined and concentrated.

¹H NMR (CD₃OD, 500 Hz): δ 8.48 (s, 1H), 8.23 (d, J=2.3 Hz, 1H), 7.64-7.65 (m, 1H), 7.63 (s, 1H), 7.20-7.32 (m, 5H), 7.01 (t, J=7.5 Hz, 1H), 6.68 (d, J=8.3 Hz, 1H), 6.45 (t, J=6.5 Hz, 1H), 6.35 (d, J=7.7 Hz, 1H), 5.67 (d, J=3.9 Hz, 1H), 4.45 (d, J=2.3 Hz, 1H), 4.32-4.35 (m, 1H), 4.18 (d, J=3.0 Hz, 1H), 3.93-4.00 (m, 1H), 3.95, (s, 3H), 3.77-3.85 (m, 2H), 3.43-3.48 (m, 1H), 3.27 (t, J=5.1 Hz, 1H), 3.03 (d, J=4.4 Hz, 1H), 2.73-2.83 (m, 2H), 2.55 (t, J=8.3 Hz, 1H), 2.34-2.43 (m, 3H), 1.93-1.98 (m, 1H), 1.66 (s, 3H), 1.52 (s, 6H), 1.14(s, 3H). LC-MS (M⁺+1) (EI) 821.5.

### EXAMPLE 15

### (αR,γS,2S)-N-[(3S,4S)-3,4-dihydro-3-hydroxy-2H-1-benzopyran-4-yl]-γ-hydroxy-4-[1-[5-(5-methoxy-3-pyridinyl)-2-oxazolyl]-1-methylethyl]-α-(phenylmethyl)-2-[[(2,2,2-trifluoroethyl)amino]carbonyl]-1-piperazinepentanamide (Compound 23a)

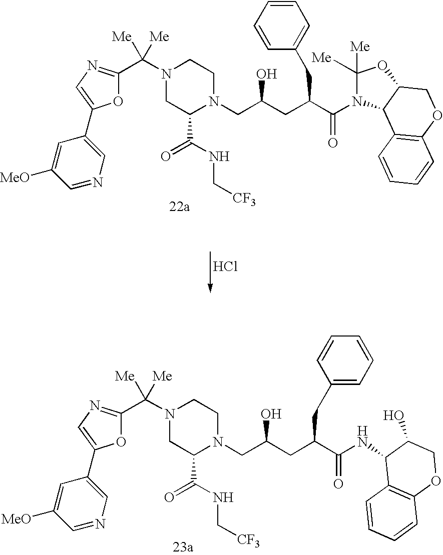

Compound 22a penultimate prepared in Example 14 (97.5 g) was dissolved in 225 mL MeOH and cooled to −10° C. 5.02N HCl in methanol (245 mL) was added dropwise over 30 minutes, keeping the temperature below 0° C. It was then transferred to a 0° C. bath. After stirring for 13 h, it was assayed and found to be greater than 98.5% complete. 5N NaOH (250 mL) was added, keeping the temperature below 0° C. After addition was complete the pH was checked and found to be 9. IPAc (1.0L) and water (200 mL) were added and the layers were shaken to dissolve a brown oil that formed during the quench. The layers were cut and the aqueous layer was assayed and found to contain 0. 19 g of Compound 23a. The organic layer was washed with 200 mL brine. The organic layer was assayed and found to contain 85.95 g of Compound 23a free base (92.7%). The brine layer was found to contain 0.05 g of Compound 23a. Activated carbon (17.99 g) was added and the mixture was stirred at 50° C. for 1 h. After cooling to room temperature the slurry was filtered through solka-floc and the cake washed with IPAc, 3×180 mL. The filtrate and washes were combined and assayed which showed 80.54 g of Compound 23a free base. The combined filtrate and washes were then concentrated to a yellow foamy solid.

¹H NMR (CD₃OD, 500 Hz): δ 8.49 (s, 1H), 8.22 (d, J=1.6 Hz, 1H), 7.66-7.67 (m, 1H), 7.20-7.25 (m, 4H), 7.14-7.17 (m, 1H), 7.06-7.10 (in, 2H), 6.80 (t, J=7.6 Hz, 1H), 6.71 (d, J=8.0 Hz, 1H), 5.13 (d, J=3.8 Hz, 1H), 4.04-4.06 (in, 2H), 3.92-3.98 ( in, 1H), 3.94 (s, 3H), 3.78-3.82 (m, 1H), 3.72-3.77 (m, 2H), 3.06-3.10 (in, 1H), 2.96-3.03 (in, 2H), 2.88-2.94 (m, 1H), 2.85 (d, J=11.2 Hz, 1H), 2.70-2.77 (in, 2H), 2.63-2.67 (in, 1H), 2.44-2.50 (in, 1H), 2.34-2.44 (in, 4H), 2.00-2.04 (in, 1H), 1.60 (s, 3H), 1.59 (s, 3H), 1.35-1.38 (m, 1H). LC-MS (M⁺+1) (EI) 781.5.

While the foregoing specification teaches the principles of the present invention, with examples provided for the purpose of illustration, the practice of the invention encompasses all of the usual variations, adaptations and/or modifications that come within the scope of the following claims.

What is claimed is:

1. A compound of Formula (III): 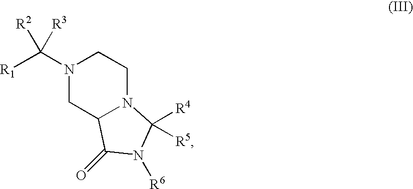
wherein:
R¹ is C₁-C₆ alkyl, C₂-C₆ alkenyl, C₂-C₆ alkynyl, C₃-C₈ cycloalkyl, aryl, or heteroaryl; wherein the alkyl, alkenyl, alkynyl, cycloalkyl, aryl or heteroaryl is optionally substituted with one or more substituents each of which is independently:
(1) cyano,
(2) C₁-C₆ alkyl,
(3) C₂-C₆ alkenyl,
(4) C₂-C₆ alkynyl,
(5) —O—C₁-C₆ alkyl,
(6) —S—C₁-C₆ alkyl,
(7) —N(R^{a})(SO₂R^{b}),
(8) —NR^{c}R^{d},
(9) —C(═O)—NR^{c}R^{d};
(10) phenyl,
(11) phenyl substituted with one or more substituents each of which is independently halogen, cyano, C₁-C₆ alkyl, C₂-C₆ alkenyl, C₂-C₆ alkynyl, —O—C₁-C₆ alkyl, or S—C₁-C₆ alkyl,
(12) heterocycle, or
(13) heterocycle substituted with one or more substituents each of which is independently cyano, C₁-C₆ alkyl, C₂-C₆ alkenyl, C₂-C₆ alkynyl, —O—C₁-C₆ alkyl, S—C₁-C₆ alkyl, NR^{c}R^{d}, or a 5- or 6-membered heteroaromatic ring consisting of from 1 to 3 heteroatoms selected from N, O and S and a balance of carbon atoms;
R² and R³ are each independently hydrogen, C₁-C₆ alkyl, or aryl, wherein the alkyl group is optionally substituted with one or more substituents each of which is independently halogen, —O—C₁-C₆ alkyl, or —O—C₁-C₆ haloalkyl; and wherein the aryl group is optionally substituted with one or more substituents each of which is independently halogen, —C₁-C₆ alkyl, —C₁-C₆ haloalkyl, —O—C₁-C₆ alkyl, or —O—C₁-C₆ haloalkyl; or
R² and R³ together with the carbon to which they are attached form C₃-C₈ cycloalkyl which is optionally substituted with one or more substituents each of which is independently halogen, —C₁-C₆ alkyl, —C₁-C₆ haloalkyl, —O—C₁-C₆ alkyl, —O—C₁-C₆ haloalkyl, or —C₁-C₆ alkyl-OR^{e};
R⁴ and R⁵ are each independently
(1) —H,
(2) halogen,
(3) —C₁-C₆ alkyl which is optionally substituted with one or more substituents each of which is independently:
(a) halogen,
(b) —O—C₁-C₆ alkyl,
(c) —O—C₁-C₆ haloalkyl,
(d) —C₁-C₆ alkyl-OR^{e}, or
(e) —N(R^{e})₂,
(4) aryl which is optionally substituted with one or more substituents each of which is independently:
(a) halogen,
(b) —O—C₁-C₆ alkyl,
(c) —O—C₁-C₆ haloalkyl,
(d) —C₁-C₆ alkyl-OR^{e}, or
(e) —N(R^{e})₂,
(5) heteroaryl which is optionally substituted with one or more substituents each of which is independently:
(a) halogen,
(b) —O—C₁-C₆ alkyl,
(c) —O—C₁-C₆ haloalkyl,
(d) —C₁-C₆ alkyl-OR^{e}, or
(e) —N(R^{e})₂,
or R⁴ and R⁵ together with the carbon atom to which they are attached form:
(i) C₃-C₈ cycloalkyl which is optionally substituted with one or more substituents each of which is independently halogen, —C₁-C₆ alkyl, —C₁-C₆ haloalkyl, —O—C₁-C₆ alkyl, —O—C₁-C₆ haloalkyl, or —C₁-C₆ alkyl-OR^{e}, or
(ii) a group of formula: 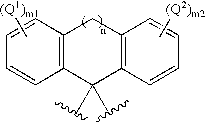
wherein each Q¹ and Q² is independently halogen, —C₁-C₆ alkyl, —C₁-C₆ haloalkyl, —O—C₁-C₆ alkyl, or —O—C₁-C₆ haloalkyl;
m1 and m2 are each independently integers equal to zero, 1, 2, 3 or 4; and
n is an integer equal to zero, 1 or 2;
R⁶ is —H or C₁-C₆ alkyl optionally substituted with one or more substituents each of which is independently
(1) halogen,
(2) —O—C₁-C₆ alkyl,
(3) —O—C₁-C₆ haloalkyl,
(4) —C₁-C₆ alkyl-OR^{e},
(5) —N(R^{e})₂,
(6) —CO₂R^{e},
(7) —N(R^{e})(SO₂R^{e}),
(8) —C(═O)R^{e}, or
(9) —C(═O)—N(R^{e})₂;
each R^{a} is independently —H or —C₁-C₄ alkyl;
each R^{b} is independently —H or —C₁-C₄ alkyl;
R^{c} and R^{d} are each independently —H or —C₁-C₄ alkyl; or alternatively R^{c} and R^{d} together with the nitrogen to which they are attached form C₃-C₆ azacycloalkyl; and
each R^{e} is independently a —C₁-C₄ alkyl; or a salt thereof.

2. The compound according to claim 1, wherein R¹ is C₁-C₆ alkyl, C₃-C₈ cycloalkyl, aryl, or heteroaryl, wherein heteroaryl is (i) a 5- or 6-membered aromatic ring consisting of from 1 to 3 heteroatoms selected from N, S, and O and a balance of carbon atoms or (ii) an 8- to 10-membered bicyclic ring system consisting of from 1 to 3 heteroatoms selected from N, S, and O and a balance of carbon atoms, wherein at least one of the rings in the bicyclic system is an aromatic ring; and wherein the alkyl, cycloalkyl, aryl or heteroaryl is optionally substituted with one or more substituents each of which is independently:
(1) cyano,
(2) C₁-C₆ alkyl,
(3) C₂-C₆ alkenyl,
(4) C₂-C₆ alkynyl,
(5) —O—C₁-C₆ alkyl,
(6) S—C₁-C₆ alkyl,
(7) —N(R^{a})(SO₂R^{b}),
(8) —NR^{c}R^{d},
(9) —C(═O)—NR^{c}R^{d};
(10) phenyl,
(11) phenyl substituted with one or more substituents each of which is independently halogen, cyano, C₁-C₆ alkyl, C₂-C₆ alkenyl, C₂-C₆ alkynyl, —O—C₁-C₆ alkyl, or S—C₁-C₆ alkyl,
(12) heterocycle, or
(13) heterocycle substituted with one or more substituents each of which is independently cyano, C₁-C₆ alkyl, C₂-C₆ alkenyl, C₂-C₆ alkynyl, —O—C₁-C₆ alkyl, S—C₁-C₆ alkyl, NR^{c}R^{d}, or a 5- or 6-membered heteroaromatic ring consisting of from 1 to 3 heteroatoms selected from N, O and S and a balance of carbon atoms;
or a salt thereof.

3. The compound according to claim 1, wherein R¹ is 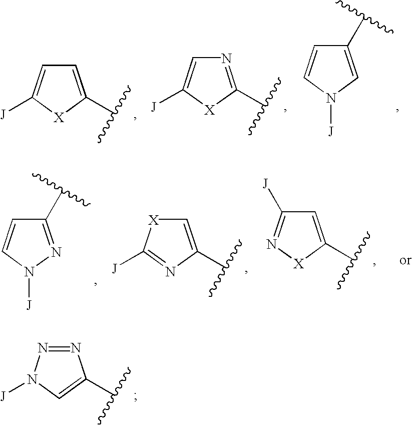
J is 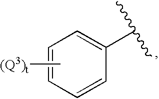
heterocycle, or substituted heterocycle;
each Q³ is independently hydrogen, halogen, cyano, C₁-C₄ alkyl, or —O—C₁-C₄ alkyl;
heterocycle is 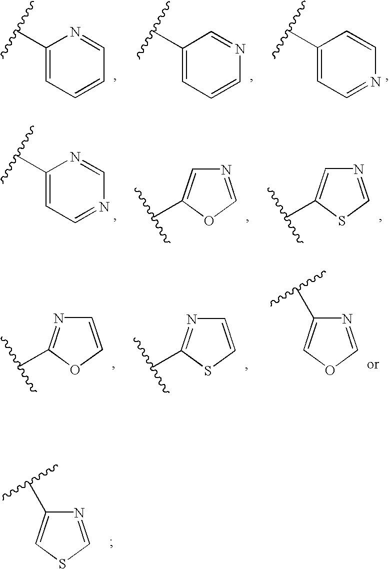
substituted heterocycle is heterocycle as defined above having one or more substituents independently selected from C₁-C₄ alkyl, —O—C₁-C₄ alkyl, —S—CH₃, —N(CH₃)₂, thiazolyl, and oxazolyl;
X is O or S; and
t is an integer equal to zero, 1 or 2;
or a salt thereof.

4. The compound according to claim 3, wherein R¹ is 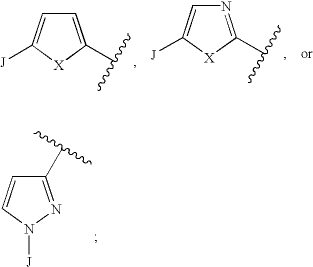
J is 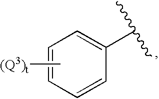
heterocycle, or substituted heterocycle;
each Q³ is independently hydrogen, halogen, cyano, C₁-C₄ alkyl, or —O—C₁-C₄ alkyl;
heterocycle is 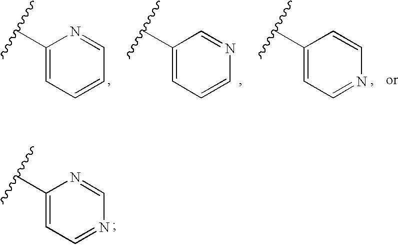
substituted heterocycle is heterocycle as defined above having one or more substituents independently selected from C₁-C₄ alkyl, —O—C₁-C₄ alkyl, —S—CH₃, —N(CH₃)₂, thiazolyl, and oxazolyl; and
X is O or S; and
t is an integer equal to zero, 1 or 2;
or a salt thereof.

5. The compound according to claim 4, wherein R¹ is 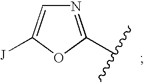
or a salt thereof.

6. The compound according to claim 1, wherein R⁶ is C₁-C₆ alkyl optionally substituted with one or more halogens each of which is independently fluoro, chloro, or bromo;
or a salt thereof.

7. The compound according to claim 6, wherein R⁶ is C₁-C₄ alkyl or C₁-C₄ fluoroalkyl;
or a salt thereof.

8. The compound according to claim 7, wherein R⁶ is 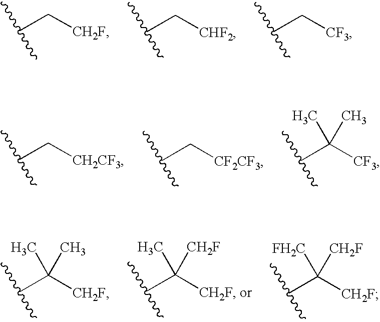
or a salt thereof.

9. The compound according to claim 8, wherein R⁶ is 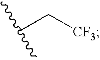
or a salt thereof.

10. The compound according to claim 1, wherein R⁴ and R⁵ are each independently —C₁-C₄ alkyl which is optionally substituted with one or more substituents each of which is independently:
(a) halogen,
(b) —O—C₁-C₄ alkyl,
(c) —O—C₁-C₄ haloalkyl,
(d) —C₁-C₄ alkyl-OR^{e}, or
(e) —N(R^{e})₂;
or a salt thereof.

11. The compound according to claim 10, wherein R⁴ and R⁵ are both methyl;
or a salt thereof.

12. The compound according to claim 1, wherein R² and R³ are either both —H or both methyl;
or a salt thereof.

13. A compound of Formula (II-Al): 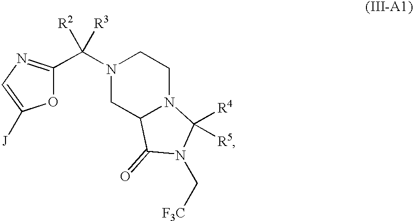
wherein
J is 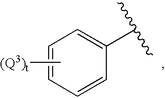
heterocycle, or substituted heterocycle;
heterocycle is 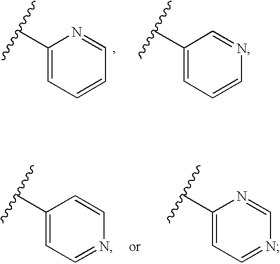
substituted heterocycle is heterocycle as defined above having one or more substituents independently selected from C₁-C₄ alkyl, —O—C₁-C₄ alkyl, —S—CH₃, —N(CH₃)₂, thiazolyl, and oxazolyl; and
each Q³ is independently hydrogen, halogen, cyano, C₁-C₄ alkyl, or —O—C₁-C₄ alkyl;
t is an integer equal to zero, 1 or 2;
R² and R³ are each independently hydrogen or C₁-C₄ alkyl; and
R⁴ and R⁵ are each independently —C₁-C₄ alkyl which is optionally substituted with one or more substituents each of which is independently:
(a) halogen,
(b) —O—C₁-C₄ alkyl,
(c) —O—C₁-C₄ haloalkyl,
(d) —C₁-C₄ alkyl-OR^{e}, or
(e) —N(R^{e})₂;
or a salt thereof.

14. The compound according to claim 13, which is a compound of Formula (III-A2): 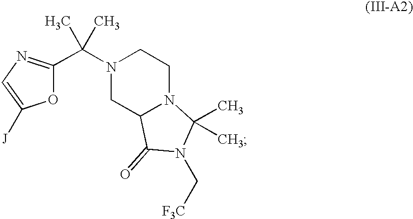
or a salt thereof.

15. The compound according to claim 14, which is Compound 13: 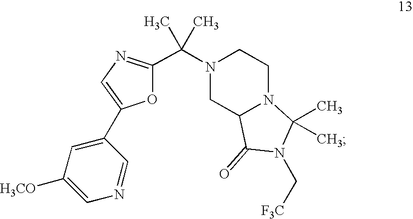
or a salt thereof.

16. A process for preparing a compound of Formula (III): 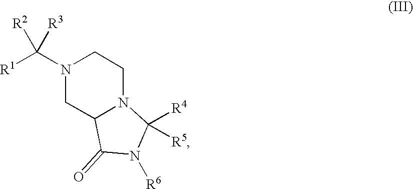
which comprises:
(C) coupling an iminium salt of Formula I: 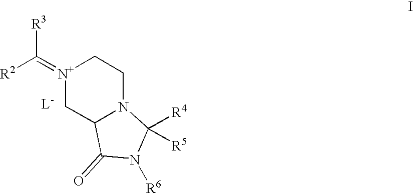
with a metallated derivative of a compound of Formula (II):
R¹—H (II),
in solvent to obtain Compound III;
wherein
L⁻ is a counterion;
R¹ is C₁-C₆ alkyl, C₂-C₆ alkenyl, C₂-C₆ alkynyl, C₃-C₈ cycloalkyl, aryl, or heteroaryl; wherein the alkyl, alkenyl, alkynyl, cycloalkyl, aryl or heteroaryl is optionally substituted with one or more substituents each of which is independently:
(1) cyano,
(2) C₁-C₆ alkyl,
(3) C₂-C₆ alkenyl,
(4) C₂-C₆ alkynyl,
(5) —O—C₁-C₆ alkyl,
(6) —S—C₁-C₆ alkyl,
(7) —N(R^{a})(SO₂R^{b}),
(8) —NR^{c}R^{d},
(9) —C(═O)—NR^{c}R^{d};
(10) phenyl,
(11) phenyl substituted with one or more substituents each of which is independently halogen, cyano, C₁-C₆ alkyl, C₂-C₆ alkenyl, C₂-C₆ alkynyl, —O—C₁-C₆ alkyl, or S—C₁-C₆ alkyl,
(12) heterocycle, or
(13) heterocycle substituted with one or more substituents each of which is independently cyano, C₁-C₆ alkyl, C₂-C₆ alkenyl, C₂-C₆ alkynyl, —O—C₁-C₆ alkyl, S—C₁-C₆ alkyl, NR^{c}R^{d}, or a 5- or 6-membered heteroaromatic ring consisting of from 1 to 3 heteroatoms selected from N, O and S and a balance of carbon atoms;
R² and R³ are each independently hydrogen, C₁-C₆ alkyl, or aryl, wherein the alkyl group is optionally substituted with one or more substituents each of which is independently halogen, —O—C₁-C₆ alkyl, or —O—C₁-C₆ haloalkyl; and wherein the aryl group is optionally substituted with one or more substituents each of which is independently halogen, —C₁-C₆ alkyl, —C₁-C₆ haloalkyl, —O—C₁-C₆ alkyl, or —O—C₁-C₆ haloalkyl; or
R² and R³ together with the carbon to which they are attached form C₃-C₈ cycloalkyl which is optionally substituted with one or more substituents each of which is independently halogen, —C₁-C₆ alkyl, —C₁-C₆ haloalkyl, —O—C₁-C₆ alkyl, —O—C₁-C₆ haloalkyl, or —C₁-C₆ alkyl-OR^{e};
R⁴ and R⁵ are each independently
(1) —H,
(2) halogen,
(3) —C₁-C₆ alkyl which is optionally substituted with one or more substituents each of which is independently:
(a) halogen,
(b) —O—C₁-C₆ alkyl,
(c) —O—C₁-C₆ haloalkyl,
(d) —C₁-C₆ alkyl-OR^{e}, or
(e) —N(R^{e})₂,
(4) aryl which is optionally substituted with one or more substituents each of which is independently:
(a) halogen,
(b) —O—C₁-C₆ alkyl,
(c) —O—C₁-C₆ haloalkyl,
(d) —C₁-C₆ alkyl-OR^{e}, or
(e) —N(R^{e})₂,
(5) heteroaryl which is optionally substituted with one or more substituents each of which is independently:
(a) halogen,
(b) —O—C₁-C₆ alkyl,
(c) —O—C₁-C₆ haloalkyl,
(d) —C₁-C₆ alkyl-OR^{e}, or
(e) —N(R^{e})₂,
or R⁴ and R⁵ together with the carbon atom to which they are attached form:
(i) C₃-C₈ cycloalkyl which is optionally substituted with one or more substituents each of which is independently halogen, —C₁-C₆ alkyl, —C₁-C₆ haloalkyl, —O—C₁-C₆ alkyl, —O—C₁-C₆ haloalkyl, or —C₁-C₆ alkyl-OR^{e}, or
(ii) a group of formula: 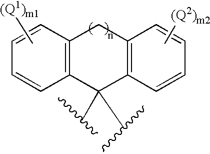
wherein each Q¹ and Q² is independently halogen, —C₁-C₆ alkyl, —C₁-C₆ haloalkyl, —O—C₁-C₆ alkyl, or —O—C₁-C₆ haloalkyl;
m1 and m2 are each independently integers equal to zero, 1, 2, 3 or 4; and
n is an integer equal to zero, 1 or 2;
R⁶ is —H or C₁-C₆ alkyl optionally substituted with one or more substituents each of which is independently
(1) halogen,
(2) —O—C₁-C₆ alkyl,
(3) —O—C₁-C₆ haloalkyl,
(4) —C₁-C₆ alkyl-OR^{e},
(5) —N(R^{e})₂,
(6) —CO₂R^{e},
(7) —N(R^{e})(SO₂R^{e}),
(8) —C(═O)R^{e}, or
(9) —C(═O)—N(R^{e})₂;
each R^{a} is independently —H or —C₁-C₄ alkyl;
each R^{b} is independently —H or —C₁-C₄ alkyl;
R^{c} and R^{d} are each independently —H or —C₁-C₄ alkyl; or alternatively R^{c} and R^{d} together with the nitrogen to which they are attached form C₃-C₆ azacycloalkyl; and
each R^{e} is independently a —C₁-C₄ alkyl.

17. The process according to claim 16, wherein R¹ is C₁-C₆ alkyl, C₃-C₈ cycloalkyl, aryl, or heteroaryl, wherein heteroaryl is (i) a 5- or 6-membered aromatic ring consisting of from 1 to 3 heteroatoms selected from N, S, and O and a balance of carbon atoms or (ii) an 8- to 10-membered bicyclic ring system consisting of from 1 to 3 heteroatoms selected from N, S, and O and a balance of carbon atoms, wherein at least one of the rings in the bicyclic system is an aromatic ring; and wherein the alkyl, cycloalkyl, aryl or heteroaryl is optionally substituted with one or more substituents each of which is independently:
(1) cyano,
(2) C₁-C₆ alkyl,
(3) C₂-C₆ alkenyl,
(4) C₂-C₆ alkynyl,
(5) —O—C₁-C₆ alkyl,
(6) S—C₁-C₆ alkyl,
(7) —N(R^{a})(SO₂R^{b}),
(8) —NR^{c}R^{d},
(9) —C(═O)—NR^{c}R^{d};
(10) phenyl,
(11) phenyl substituted with one or more substituents each of which is independently halogen, cyano, C₁-C₆ alkyl, C₂-C₆ alkenyl, C₂-C₆ alkynyl, —O—C₁-C₆ alkyl, or S—C₁-C₆ alkyl,
(12) heterocycle, or
(13) heterocycle substituted with one or more substituents each of which is independently cyano, C₁-C₆ alkyl, C₂-C₆ alkenyl, C₂-C₆ alkynyl, —O—C₁-C₆ alkyl, S—C₁-C₆ alkyl, NR^{c}R^{d}, or a 5- or 6-membered heteroaromatic ring consisting of from 1 to 3 heteroatoms selected from N, O and S and a balance of carbon atoms.

18. The process according to claim 17, wherein R¹ is 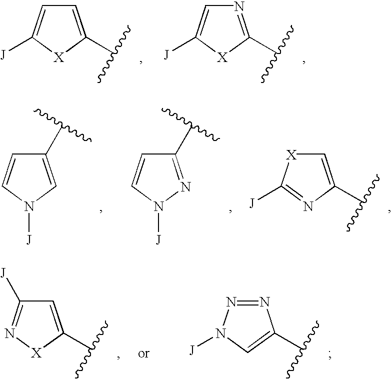
J is 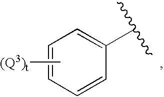
heterocycle, or substituted heterocycle;
heterocycle is 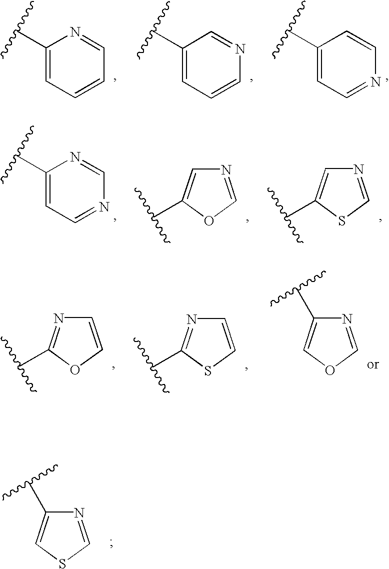
substituted heterocycle is heterocycle as defined above having one or more substituents independently selected from C₁-C₄ alkyl, —O—C₁-C₄ alkyl, —S—CH₃, —N(CH₃)₂, thiazolyl, and oxazolyl;
each Q³ is independently hydrogen, halogen, cyano, C₁-C₄ alkyl, or —O—C₁-C₄ alkyl;
X is O or S; and
t is an integer equal to zero, 1 or 2.

19. The process according to claim 18, wherein R¹ is 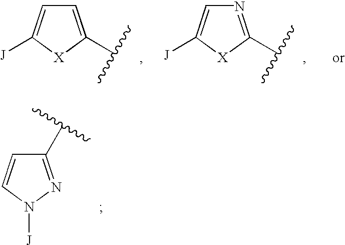
J is 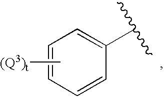
heterocycle, or substituted heterocycle;
heterocycle is 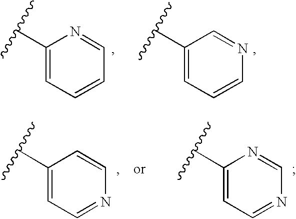
substituted heterocycle is heterocycle as defined above having one or more substituents independently selected from C₁-C₄ alkyl, —O—C₁-C₄ alkyl, —S—CH₃, —N(CH₃)₂, thiazolyl, and oxazolyl;
each Q³ is independently hydrogen, halogen, cyano, C₁-C₄ alkyl, or —O—C₁-C₄ alkyl;
X is O or S; and
t is an integer equal to zero, 1 or 2.

20. The process according to claim 19, wherein R¹ is

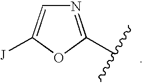

21. The process according to claim 16, wherein R⁶ is C₁-C₄ alkyl or C₁-C₄ fluoroalkyl.

22. The process according to claim 21, wherein R⁶ is

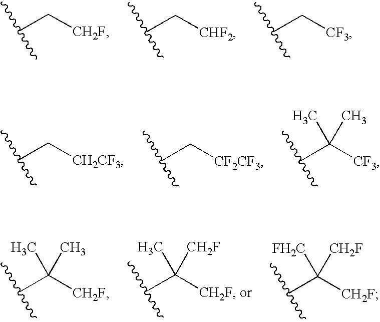

23. The process according to claim 22, wherein R⁶ is

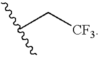

24. The process according to claim 16, wherein R⁴ and R⁵ are each independently —C₁-C₄ alkyl which is optionally substituted with one or more substituents each of which is independently:
(a) halogen,
(b) —O—C₁-C₄ alkyl,
(c) —O—C₁-C₄ haloalkyl,
(d) —C₁-C₄ alkyl-OR^{e}, or
(e) —N(R^{e})₂.

25. The process according to claim 24, wherein R⁴ and R⁵ are both methyl.

26. The process according to claim 16, wherein R² and R³ are each independently hydrogen or C₁-C₄ alkyl.

27. The process according to claim 26, wherein R² and R³ are either both —H or both methyl.

28. The process according to claim 16, wherein the metallated derivative is prepared by treating Compound II with a metal-containing deprotonating agent.

29. The process according to claim 16, wherein L⁻ is selected from the group consisting of:
(1) halide,
(2) cyanide,
(3) BF₄⁻,
(4) (C₆F₅)₄B⁻,
(5) MF₆⁻, wherein M is P, As, or Sb,
(6) ClO₄⁻,
(7) benzotriazolyl anion,
(8) aryl-SO₃⁻, wherein the aryl is optionally substituted with one or more substituents each of which is independently halo, C₁-C₁₀ alkyl, or C₁-C₁₀ haloalkyl,
(9) C₁-C₆ alkyl-SO₃— wherein the alkyl is optionally substituted with one or more halogens, and
(10) trihaloacetate anion.

30. The process according to claim 29, wherein L⁻ is selected from the group consisting of fluoride, chloride, cyanide, BF₄⁻, (C₆F₅)₄B⁻, PF₆⁻, ClO₄⁻, benzotriazolyl anion, OTf⁻, CF₃CF₂SO₃⁻, C₆F₅SO₃⁻, OTs⁻, and CF₃CO₂⁻.

31. The process according to claim 16, wherein the coupling reaction is conducted at a temperature in the range of from about −80 to about 20° C.

32. The process according to claim 16, wherein the metallated derivative of Compound II is present in an amount in the range of from about 0.9 to about 3 equivalents per equivalent of Compound I.

33. The process according to claim 16, which is a process for preparing a compound of Formula (III-A2): 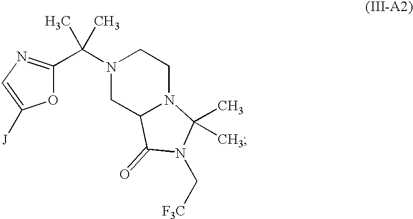
which comprises:
(C) coupling an iminium salt of Formula (I-A): 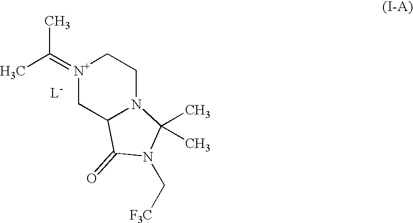
with a metallated derivative of a compound of Formula (II-A): 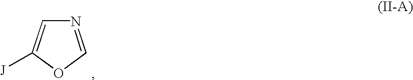
in solvent to obtain compound II-A2;
wherein
L⁻ is a counterion;
J is 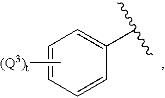
heterocycle, or substituted heterocycle;
heterocycle is 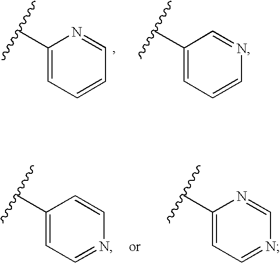
substituted heterocycle is heterocycle as defined above having one or more substituents independently selected from C₁-C₄ alkyl, —O—C₁-C₄ alkyl, —S—CH₃, —N(CH₃)₂, thiazolyl, and oxazolyl;
each Q³ is independently hydrogen, halogen, cyano, C₁-C₄ alkyl, or —O—C₁-C₄ alkyl; and
t is an integer equal to zero, 1 or 2.

34. The process according to claim 16, which further comprises:
(D) treating Compound III with acid to obtain a compound of Formula (IV):

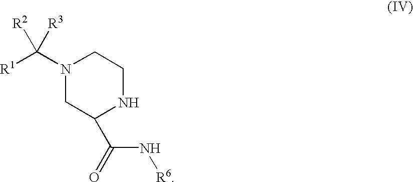

35. The process according to claim 34, which further comprises:
(E) reacting piperazine carboxamide IV with an epoxide of Formula (V): 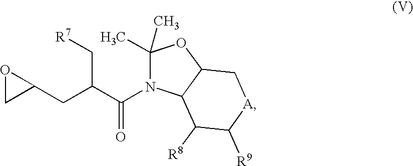
to obtain a compound of Formula (VI): 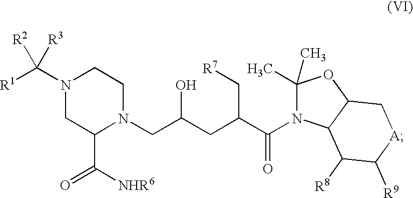
wherein
A is absent, CH₂, O, or S;
R⁷ is C₁-C₆ alkyl, C₃-C₆ cycloalkyl, aryl, or heteroaryl; wherein the alkyl or cycloalkyl is optionally substituted with one or more substituents each of which is independently halogen, hydroxy, —C₁-C₆ haloalkyl, —O—C₁-C₆ alkyl, or —O—C₁-C₆ haloalkyl; and wherein aryl or heteroaryl is optionally substituted with one or more substituents each of which is independently halogen, hydroxy, —C₁-C₆ haloalkyl, —O—C₁-C₆ alkyl, —O—C₁-C₆ haloalkyl, C₂-C₆ alkenyl, or C₂-C₆ alkynyl; and
R⁸ and R⁹ are each independently —H, —C₁-C₆ alkyl, —C₁-C₆ haloalkyl, —C₃-C₆ cycloalkyl, or aryl, wherein the aryl is optionally substituted with one or more substituents each of which is independently halogen, —OH, —C₁-C₆ alkyl, —C₁-C₆ haloalkyl, —O—C₁-C₆ alkyl, or —O—C₁-C₆ haloalkyl; or alternatively
R⁸ and R⁹ together with the carbons to which each is attached form a fused benzene ring which is optionally substituted with one or more substituents each of which is independently halogen, —OH, —C₁-C₆ alkyl, —C₁-C₆ haloalkyl, —O—C₁-C₆ alkyl, or —O—C₁-C₆ haloalkyl.

36. The process according to claim 35, which further comprises:
(F) treating Compound VI with acid to obtain a compound of Formula (VII):

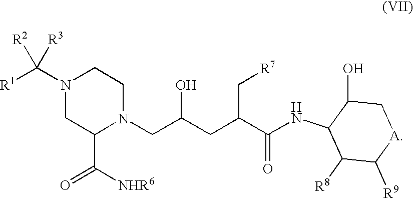

37. The process according to claim 35, wherein R⁷ is

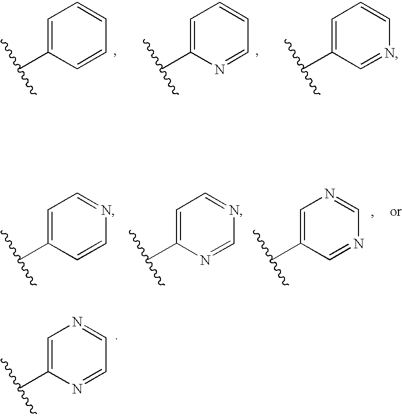

38. The process according to claim 35, wherein
A is absent or O; and
R⁸ and R⁹ together with the carbons to which each is attached form a fused benzene ring which is optionally substituted with 1 or 2 substituents each of which is independently independently halogen, —C₁-C₄ alkyl, —C₁-C₄ fluoroalkyl, —O—C₁-C₄ alkyl, or —O—C₁-C₄ fluoroalkyl.

39. A process for preparing Compound 13: 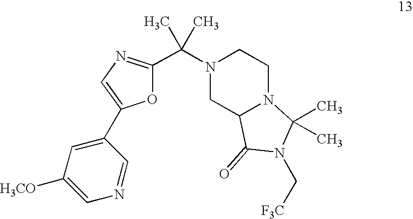
which comprises:
(cc) coupling Compound 10: 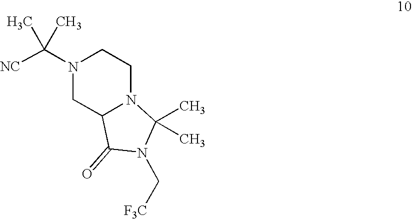
in the presence of a Lewis acid, or coupling compound I-A: 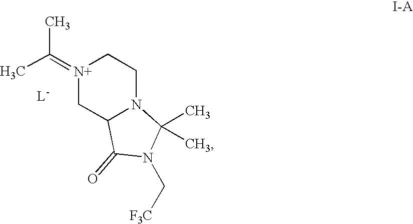
with a metallated derivative of Compound 4: 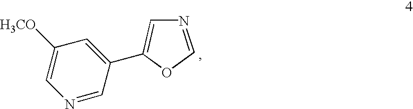
in solvent to obtain 13;
wherein L⁻ is a non-nucleophilic counterion.

40. The process according to claim 39, wherein the metallated derivative is prepared by treating 4 with a deprotonating agent selected from the group consisting of C₁-C₆ alkyllithiums, C₆-C₁₀ aryllithiums, and C₁-C₆ alkylmagnesium halides.

41. The process according to claim 39, wherein L⁻ is selected from the group consisting of:
(1) halide,
(2) BF₄⁻,
(3) (C₆F₅)₄B⁻,
(4) MF₆⁻, wherein M is P, As, or Sb,
(5) ClO₄⁻,
(6) benzotriazolyl anion,
(7) aryl-SO₃⁻, wherein the aryl is optionally substituted with one or more substituents each of which is independently halo, C₁-C₁₀ alkyl, or C₁-C₁₀ haloalkyl,
(8) C₁-C₆ alkyl-SO₃⁻ wherein the alkyl is optionally substituted with one or more halogens, and
(9) trihaloacetate anion.

42. The process according to claim 39, wherein
the solvent employed in Step (cc) is an ether;
the coupling reaction is conducted at a temperature in the range of from about −80 to about 20° C.; and
the metallated derivative of 4 is present in an amount in the range of from about 1 to about 2 equivalents per equivalent of 10 or of I-A.

43. The process according to claim 39, which further comprises:
(dd) treating Compound 13 with acid to obtain Compound 15: 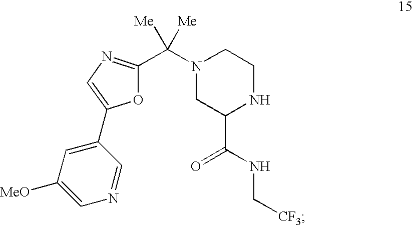
(ee) reacting piperazine carboxamide 13 with epoxide 21: 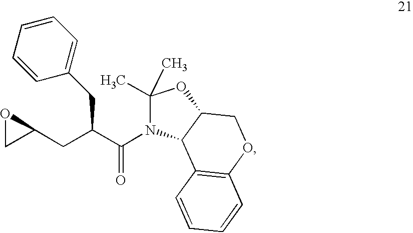
to obtain Compound 22:

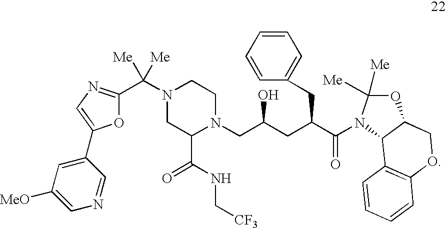

44. The process according to claim 43, which further comprises:
(ff) treating Compound 22 with acid to obtain Compound 23:

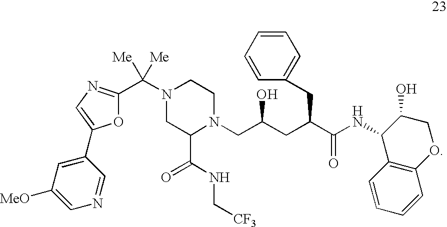

45. A process for preparing an iminium salt of Formula (I): 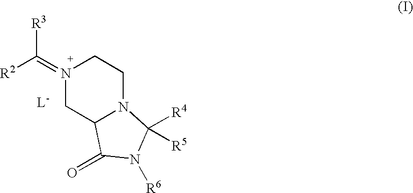
which comprises:
(A) reacting a piperazine of Formula (VIII): 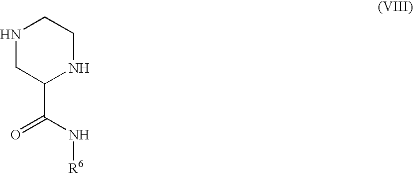
with a carbonyl-containing compound of Formula (IX): 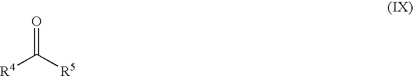
optionally in the presence of at least a catalytic amount of an acid to form an acetonide of Formula (X): 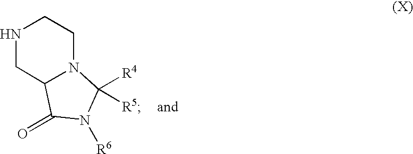
(B) reacting the acetonide of Formula (X) with (i) HL and a carbonyl-containing compound of Formula (XI): 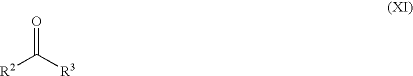
or a ketal of Formula (XI-A): 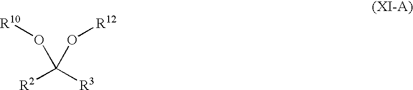
or (ii) with an alcohol of Formula (XII): 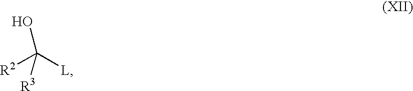
to form Compound I;
wherein
L⁻ is a counterion;
R² and R³ are each independently hydrogen, C₁-C₆ alkyl, or aryl, wherein the alkyl or aryl group is optionally substituted with one or more substituents each of which is independently halogen, —O—C₁-C₆ alkyl, or —O—C₁-C₆ haloalkyl; or R² and R³ together with the carbon to which they are attached form C₃-C₈ cycloalkyl which is optionally substituted with one or more substituents each of which is independently halogen, —O—C₁-C₆ alkyl, —O—C₁-C₆ haloalkyl, or —C₁-C₆ alkyl-OR^{a};
R⁴ and R⁵ are each independently
(1) —H,
(2) halogen,
(3) —C₁-C₆ alkyl which is optionally substituted with one or more substituents each of which is independently:
(a) halogen,
(b) —O—C₁-C₆ alkyl,
(c) —O—C₁-C₆ haloalkyl,
(d) —C₁-C₆ alkyl-OR^{e}, or
(e) —N(R^{e})₂,
(4) aryl which is optionally substituted with one or more substituents each of which is independently:
(a) halogen,
(b) —O—C₁-C₆ alkyl,
(c) —O—C₁-C₆ haloalkyl,
(d) —C₁-C₆ alkyl-OR^{e}, or
(e) —N(R^{e})₂,
(5) heteroaryl which is optionally substituted with one or more substituents each of which is independently:
(a) halogen,
(b) —O—C₁-C₆ alkyl,
(c) —O—C₁-C₆ haloalkyl,
(d) —C₁-C₆ alkyl-OR^{e}, or
(e) —N(R^{e})₂,
or R⁴ and R⁵ together with the carbon atom to which they are attached form (i) C₃-C₈ cycloalkyl which is optionally substituted with one or more substituents each of which is independently halogen, —O—C₁-C₆ alkyl, —O—C₁-C₆ haloalkyl, or —C₁-C₆ alkyl-OR^{e} or (ii) a group of formula: 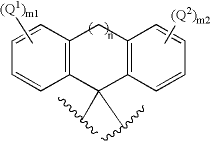
wherein each Q¹ and Q² is independently halogen, —C₁-C₆ alkyl, —C₁-C₆ haloalkyl, —O—C₁-C₆ alkyl, or —O—C₁-C₆ haloalkyl; m1 and m2 are each independently integers equal to zero, 1, 2, 3 or 4; and n is an integer equal to zero, 1 or 2;
R⁶ is —H or C₁-C₆ alkyl optionally substituted with one or more substituents each of which is independently
(1) halogen,
(2) —O—C₁-C₆ alkyl,
(3) —O—C₁-C₆ haloalkyl,
(4) —C₁-C₆ alkyl-OR^{e},
(5) —N(R^{e})₂,
(6) —CO₂R^{e},
(7) —N(R^{e})(SO₂R^{e}),
(8) —C(═O)R^{e}, or
(9) —C(═O)—N(R^{e})₂;
R¹⁰ and R¹² are each independently C₁-C₄ alkyl; and
R^{e} is —C₁-C₄ alkyl.

46. The process according to claim 45, wherein L⁻ is selected from the group consisting of:
(1) halide,
(2) cyanide,
(3) BF₄⁻,
(4) (C₆F₅)₄B⁻,
(5) MF₆⁻, wherein M is P, As, or Sb,
(6) ClO₄⁻,
(7) benzotriazolyl anion,
(8) aryl-SO₃⁻, wherein the aryl is optionally substituted with one or more substituents each of which is independently halo, C₁-C₁₀ alkyl, or C₁-C₁₀ haloalkyl,
(9) C₁-C₆ alkyl-SO₃⁻ wherein the alkyl is optionally substituted with one or more halogens, and
(10) trihaloacetate anion.

47. The process according to claim 45, wherein R⁶ is C₁-C₄ alkyl or C₁-C₄ fluoroalkyl.

48. The process according to claim 47, wherein R⁶ is

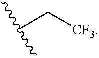

49. The process according to claim 45, wherein R⁴ and R⁵ are each independently —C₁-C₄ alkyl which is optionally substituted with one or more substituents each of which is independently:
(a) halogen,
(b) —O—C₁-C₄ alkyl,
(c) —O—C₁-C₄ haloalkyl,
(d) —C₁-C₄ alkyl-OR^{e}, or
(e) —N(R^{e})₂.

50. The process according to claim 49, wherein R⁴ and R⁵ are both methyl.

51. The process according to claim 45, wherein R² and R³ are each independently hydrogen or C₁-C₄ alkyl.

52. The process according to claim 51, wherein R² and R³ are both methyl.

53. The process according to claim 45, wherein Step (B) comprises treating the acetonide of Formula (X) with HL to form the acid addition salt thereof and then reacting the addition salt with acetonide XI or ketal XI-A.

54. The process according to claim 45, wherein Step A is conducted at a temperature in the range of from about 0 to about 200° C., and Step B is conducted at a temperature in the range of from about 0 to about 200° C.

55. The process according to claim 45, wherein in Step A piperazine carboxamide VIII is present in an amount in the range of from about 0.001 to about 10 equivalent per equivalent of Compound IX; and wherein in Step B (i) Compound XI or XI-A and HL are present in equivalent amounts and each is present in an amount in the range of from about 0.5 to about 10 equivalents per equivalent of acetonide X, or (ii) Compound XII is present in an amount in the range of from about 0.5 to about 10 equivalents per equivalent of acetonide X.

56. The process according to claim 45, wherein the process is conducted in one pot by adding (i) Compound XI or XI-A and HL or (ii) alcohol XII to the pot before commencement of, during, or after completion of reaction step A, wherein
(a) when the addition of (i) Compound XI or XI-A and HL or (ii) alcohol XII to the pot is before commencement of reaction step A, reaction steps A and B are conducted concurrently in the pot;
(b) when the addition of (i) Compound XI or XI-A and HL or (ii) alcohol XII to the pot is during reaction step A, reaction steps A and B are conducted concurrently in the pot subsequent to the addition; and
(c) when the addition of (i) Compound XI or XI-A and HL or (ii) alcohol XII to the pot is after completion of reaction step A, reaction steps A and B are conducted sequentially in the pot.

57. A process for preparing an iminium salt of Formula (I-A): 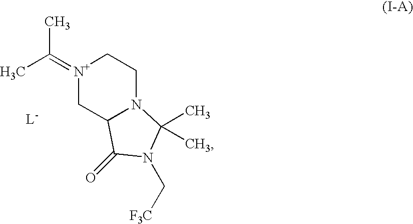
which comprises:
(aa) reacting a piperazine carboxamide 7: 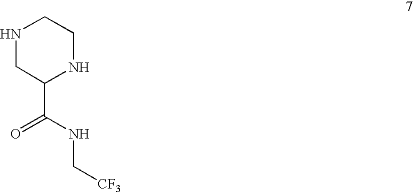
with acetone to form an acetonide 9: 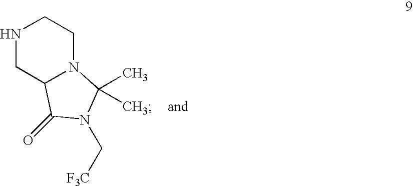
(bb) reacting acetonide 9 with HL and acetone or a ketal of acetone to form I-A;
wherein L⁻ is a non-nucelophilic counterion.

58. The process according to claim 57, wherein L⁻ is selected from the group consisting of:
(1) halide,
(2) BF₄⁻,
(3) (C₆F₅)₄B⁻,
(4) MF₆⁻, wherein M is P, As, or Sb,
(5) ClO₄⁻,
(6) benzotriazolyl anion,
(7) aryl-SO₃⁻, wherein the aryl is optionally substituted with one or more substituents each of which is independently halo, C₁-C₁₀ alkyl, or C₁-C₁₀ haloalkyl,
(8) C₁-C₆ alkyl-SO₃⁻ wherein the alkyl is optionally substituted with one or more halogens, and
(9) trihaloacetate anion.

59. The process according to claim 58, wherein L⁻ is OTf⁻ and iminium salt I-A is iminium salt 11:

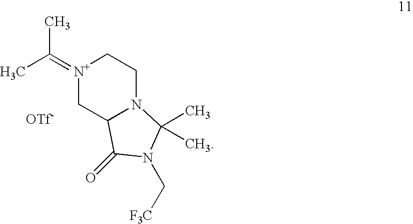

60. A process for preparing Compound 10: 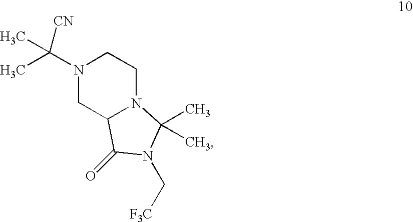
which comprises:
(A) reacting a piperazine carboxamide 8: 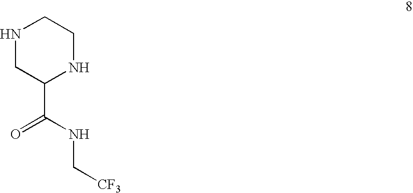
with acetone to form an acetonide 9: 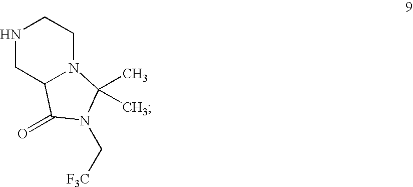
and
(B) reacting acetonide 9 with acetone cyanohydrin to form 10.

61. A compound of Formula (I-A): 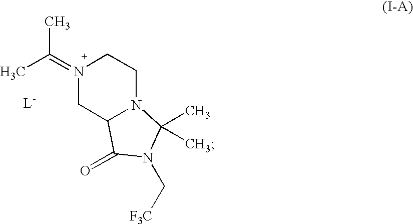
wherein L⁻ is a counterion.

62. The compound according to claim 61, which is Compound 11:

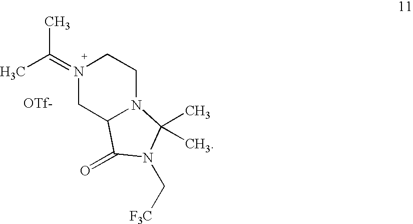

63. The compound according to claim 61, which is Compound 10:

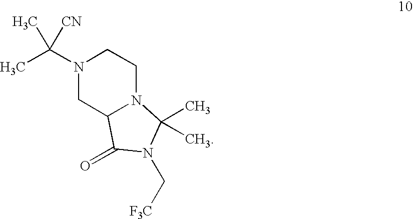

64. The compound according to claim 61, which is a compound of Formula (I-Aa):

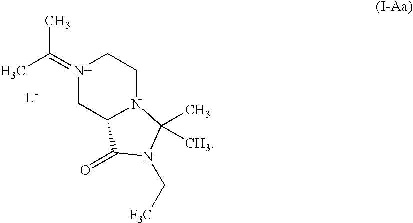

65. The compound according to claim 61, which is Compound 11a:

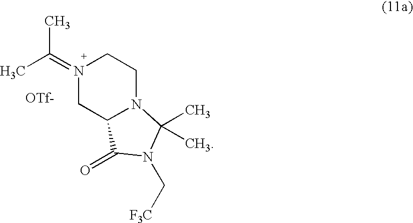

66. A compound of Formula (X): 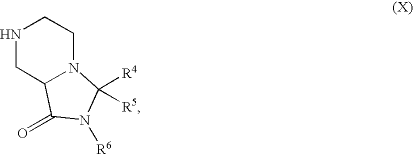
wherein R⁴ and R⁵ are each independently
(1) —H,
(2) halogen,
(3) —C₁-C₆ alkyl which is optionally substituted with one or more substituents each of which is independently:
(a) halogen,
(b) —O—C₁-C₆ alkyl,
(c) —O—C₁-C₆ haloalkyl,
(d) —C₁-C₆ alkyl-OR^{e}, or
(e) —N(R^{e})₂,
(4) aryl which is optionally substituted with one or more substituents each of which is independently:
(a) halogen,
(b) —O—C₁-C₆ alkyl,
(c) —O—C₁-C₆ haloalkyl,
(d) —C₁-C₆ alkyl-OR^{e}, or
(e) —N(R^{e})₂,
(5) heteroaryl which is optionally substituted with one or more substituents each of which is independently:
(a) halogen,
(b) —O—C₁-C₆ alkyl,
(c) —O—C₁-C₆ haloalkyl,
(d) —C₁-C₆ alkyl-OR^{e}, or
(e) —N(R^{e})₂,
or R⁴ and R⁵ together with the carbon atom to which they are attached form:
(i) C₃-C₈ cycloalkyl which is optionally substituted with one or more substituents each of which is independently halogen, —C₁-C₆ alkyl, —C₁-C₆ haloalkyl, —O—C₁-C₆ alkyl, —O—C₁-C₆ haloalkyl, or —C₁-C₆ alkyl-OR^{e}, or
(ii) a group of formula: 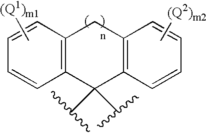
wherein each Q¹ and Q² is independently halogen, —C₁-C₆ alkyl, —C₁-C₆ haloalkyl, —O—C₁-C₆ alkyl, or —O—C₁-C₆ haloalkyl; m1 and m2 are each independently integers equal to zero, 1, 2, 3 or 4; and n is an integer equal to zero, 1 or 2;
R⁶ is —H or C₁-C₆ alkyl optionally substituted with one or more substituents each of which is independently
(1) halogen,
(2) —O—C₁-C₆ alkyl,
(3) —O—C₁-C₆ haloalkyl,
(4) —C₁-C₆ alkyl-OR^{e},
(5) —N(R^{e})₂,
(6) —CO₂R^{e},
(7) —N(R^{e})(SO₂R^{e}),
(8) —C(═O)R^{e}, or
(9) —C(═O)—N(R^{e})₂; and
each R^{e} is independently a —C₁-C₄ alkyl;
or a salt thereof.

67. The compound according to claim 66, which is Compound 9: 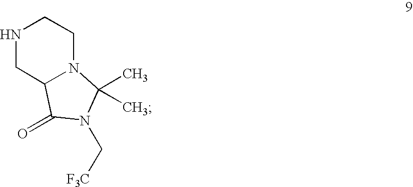
or a salt thereof.

68. The compound according to claim 66, which is a compound of Formula (Xa): 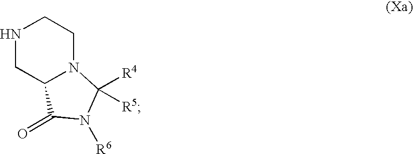
or a salt thereof.

69. The compound according to claim 66, which is Compound 9a: 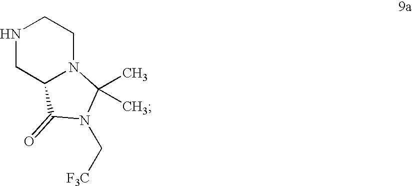
or a salt thereof.

70. An oxazole of formula (II-A), or a salt thereof: 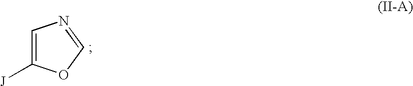
wherein J is pyridyl or pyrimidinyl, either of which is optionally substituted with from 1 to 3 substituents each of which is independently C₁-C₄ alkyl, —O—C₁-C₄ alkyl, —S—CH₃, —N(CH₃)₂, thiazolyl, or oxazolyl.

71. The compound according to claim 70, which is Compound 4: [chemical structure]
